(19) United States
(12) Patent Application Publication
(10) Pub. No.: US 2024/0327871 A1
(43) Pub. Date: Oct. 3, 2024

(54) SYSTEMS AND METHODS FOR TRANSPOSING CARGO NUCLEOTIDE SEQUENCES

(71) Applicant: Metagenomi, Inc., Emeryville, CA (US)

(72) Inventors: Brian C. THOMAS, Berkeley, CA (US); Christopher BROWN, Albany, CA (US); Daniela S.A. GOLTSMAN, Oakland, CA (US); Lisa ALEXANDER, Albany, CA (US); Sarah LAPERRIERE, Berkeley, CA (US)

(21) Appl. No.: 18/598,610

(22) Filed: Mar. 7, 2024

Related U.S. Application Data
(63) Continuation of application No. PCT/US2022/076059, filed on Sep. 7, 2022.
(60) Provisional application No. 63/241,934, filed on Sep. 8, 2021.

(51) Int. Cl. C12N 15/90; C12N 9/22

(52) U.S. Cl. CPC C12N 15/907; C12N 9/22; C12N 15/90; C12N 2800/90

(57) ABSTRACT

The present disclosure provides systems and methods for transposing a cargo nucleotide sequence to a target nucleic acid site. These systems and methods may comprise a first double-stranded nucleic acid comprising the cargo nucleotide sequence, wherein the cargo nucleotide sequence is configured to interact with a transposase, and the transposase, wherein said transposase is configured to transpose the cargo nucleotide sequence to the target nucleic acid site.

### CROSS-REFERENCE

This application is a continuation of International Application No. PCT/US2022/076059, entitled “SYSTEMS AND METHODS FOR TRANSPOSING CARGO NUCLEOTIDE SEQUENCES”, filed on Sep. 7, 2022, which claims the benefit of U.S. Provisional Patent Application No. 63/241,934, entitled “SYSTEMS AND METHODS FOR TRANSPOSING CARGO NUCLEOTIDE SEQUENCES”, filed Sep. 8, 2021, which is incorporated herein by this reference in its entirety.

### BACKGROUND

Transposable elements are movable DNA sequences which play a crucial role in gene function and evolution. While transposable elements are found in nearly all forms of life, their prevalence varies among organisms, with a large proportion of the eukaryotic genome encoding for transposable elements (at least 45% in humans). While the foundational research on transposable elements was conducted in the 1940s, their potential utility in DNA manipulation and gene editing applications has only been recognized in recent years.

### SEQUENCE LISTING

The instant application contains a Sequence Listing which has been submitted electronically in XML format and is hereby incorporated by reference in its entirety. Said XML copy, created on May 29, 2024, is named 55921-733301_SL.xml and is 475,691 bytes in size.

### SUMMARY

In some aspects, the present disclosure provides for an engineered transposase system, comprising: a double-stranded nucleic acid comprising a cargo nucleotide sequence, wherein the cargo nucleotide sequence is configured to interact with a transposase; and a transposase, wherein: the transposase is configured to transpose the cargo nucleotide sequence to a target nucleic acid locus; and the transposase is derived from an uncultivated microorganism.

In some embodiments, the transposase comprises a sequence having at least 75% sequence identity to any one of SEQ ID NOs: 1-349. In some embodiments, the transposase is not a TnpA transposase or a TnpB transposase. In some embodiments, the transposase has less than 80% sequence identity to a TnpA transposase. In some embodiments, the transposase has less than 80% sequence identity to a TnpB transposase. In some embodiments, the transposase has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 1, 3, 5, 7, 9, 11, 13, 15, and 18-19. In some embodiments, the transposase comprises a catalytic tyrosine residue. In some embodiments, the transposase is configured to bind a left-hand region comprising a subterminal palindromic sequence and a right-hand region comprising a subterminal palindromic sequence. In some embodiments, the transposase is configured to transpose the cargo nucleotide sequence as single-stranded deoxyribonucleic acid polynucleotide. In some embodiments, the transposase comprises one or more nuclear localization sequences (NLSs) proximal to an N- or C-terminus of the transposase. In some embodiments, the NLS comprises a sequence at least 80% identical to a sequence from the group consisting of SEQ ID NO: 455-470. In some embodiments, the sequence identity is determined by a BLASTP, CLUSTALW, MUSCLE, MAFFT, or CLUSTALW with the parameters of the Smith-Waterman homology search algorithm. In some embodiments, the sequence identity is determined by the BLASTP homology search algorithm using parameters of a wordlength (W) of 3, an expectation (E) of 10, and a BLOSUM62 scoring matrix setting gap costs at existence of 11, extension of 1, and using a conditional compositional score matrix adjustment.

In some aspects, the present disclosure provides for an engineered transposase system, comprising: a double-stranded nucleic acid comprising a cargo nucleotide sequence, wherein the cargo nucleotide sequence is configured to interact with a transposase; and a transposase, wherein: the transposase is configured to transpose the cargo nucleotide sequence to a target nucleic acid locus; and the transposase comprises a sequence having at least 75% sequence identity to any one of SEQ ID NOs: 1-349.

In some embodiments, the transposase is derived from an uncultivated microorganism. In some embodiments, the transposase is not a TnpA transposase or a TnpB transposase. In some embodiments, the transposase has less than 80% sequence identity to a TnpA transposase. In some embodiments, the transposase has less than 80% sequence identity to a TnpB transposase. In some embodiments, the transposase has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 1, 3, 5, 7, 9, 11, 13, 15, and 18-19. In some embodiments, the transposase comprises a catalytic tyrosine residue. In some embodiments, the transposase is configured to bind a left-hand region comprising a subterminal palindromic sequence and a right-hand region comprising a subterminal palindromic sequence. In some embodiments, the transposase is compatible with a left-hand recognition sequence or a right-hand recognition sequence. In some embodiments, the transposase is configured to transpose the cargo nucleotide sequence as single-stranded deoxyribonucleic acid polynucleotide. In some embodiments, the sequence identity is determined by a BLASTP, CLUSTALW, MUSCLE, MAFFT, or CLUSTALW with the parameters of the Smith-Waterman homology search algorithm. In some embodiments, the sequence identity is determined by the BLASTP homology search algorithm using parameters of a wordlength (W) of 3, an expectation (E) of 10, and a BLOSUM62 scoring matrix setting gap costs at existence of 11, extension of 1, and using a conditional compositional score matrix adjustment.

In some aspects, the present disclosure provides for a deoxyribonucleic acid polynucleotide encoding any engineered transposase system disclosed herein.

In some aspects, the present disclosure provides for a nucleic acid comprising an engineered nucleic acid sequence optimized for expression in an organism, wherein the nucleic acid encodes a transposase, and wherein the transposase is derived from an uncultivated microorganism, wherein the organism is not the uncultivated microorganism.

In some embodiments, the transposase comprises a variant having at least 75% sequence identity to any one of SEQ ID NOs: 1-349. In some embodiments, the transposase comprises a sequence encoding one or more nuclear localization sequences (NLSs) proximal to an N- or C-terminus of the transposase. In some embodiments, the NLS comprises a sequence selected from SEQ ID NOs: 455-470. In some embodiments, the NLS comprises SEQ ID NO: 456. In some embodiments, the NLS is proximal to the N-terminus of the transposase. In some embodiments, the NLS comprises SEQ ID NO: 455. In some embodiments, the NLS is proximal to the C-terminus of the transposase. In some embodiments, the organism is prokaryotic, bacterial, eukaryotic, fungal, plant, mammalian, rodent, or human.

In some aspects, the present disclosure provides for a vector comprising any nucleic acid disclosed herein. In some embodiments, the nucleic acid further comprises a nucleic acid encoding a cargo nucleotide sequence configured to form a complex with the transposase. In some embodiments, the vector is a plasmid, a minicircle, a CELiD, an adeno-associated virus (AAV) derived virion, or a lentivirus.

In some aspects, the present disclosure provides for a cell comprising any vector disclosed herein.

In some aspects, the present disclosure provides for a method of manufacturing a transposase, comprising cultivating any cell disclosed herein.

In some aspects, the present idsclosue provides for a method for binding, nicking, cleaving, marking, modifying, or transposing a double-stranded deoxyribonucleic acid polynucleotide comprising a cargo sequence, comprising: contacting the double-stranded deoxyribonucleic acid polynucleotide with a transposase configured to transpose the cargo nucleotide sequence to a target nucleic acid locus; and wherein the transposase comprises a sequence having at least 75% sequence identity to any one of SEQ ID NOs: 1-349.

In some embodiments, the transposase is derived from an uncultivated microorganism. In some embodiments, the transposase is not a TnpA transposase or a TnpB transposase. In some embodiments, the transposase has less than 80% sequence identity to a TnpA transposase. In some embodiments, the transposase has less than 80% sequence identity to a TnpB transposase. In some embodiments, the transposase has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity to any one of SEQ ID NOs: 1, 3, 5, 7, 9, 11, 13, 15, and 18-19. In some embodiments, the transposase comprises a catalytic tyrosine residue. In some embodiments, the transposase is configured to bind a left-hand region comprising a subterminal palindromic sequence and a right-hand region comprising a subterminal palindromic sequence. In some embodiments, the transposase is compatible with a left-hand recognition sequence or a right-hand recognition sequence. In some embodiments, the double-stranded deoxyribonucleic acid polynucleotide is transposed as a single-stranded deoxyribonucleic acid polynucleotide. In some embodiments, the double-stranded deoxyribonucleic acid polynucleotide is a eukaryotic, plant, fungal, mammalian, rodent, or human double-stranded deoxyribonucleic acid polynucleotide.

In some aspects, the present disclosure provides for a method of modifying a target nucleic acid locus, the method comprising delivering to the target nucleic acid locus an engineered transposase system disclosed herein, wherein the transposase is configured to transpose the cargo nucleotide sequence to the target nucleic acid locus, and wherein the complex is configured such that upon binding of the complex to the target nucleic acid locus, the complex modifies the target nucleic acid locus.

In some embodiments, modifying the target nucleic acid locus comprises binding, nicking, cleaving, marking, modifying, or transposing the target nucleic acid locus. In some embodiments, the target nucleic acid locus comprises deoxyribonucleic acid (DNA). In some embodiments, the target nucleic acid locus comprises genomic DNA, viral DNA, or bacterial DNA. In some embodiments, the target nucleic acid locus is in vitro. In some embodiments, the target nucleic acid locus is within a cell. In some embodiments, the cell is a prokaryotic cell, a bacterial cell, a eukaryotic cell, a fungal cell, a plant cell, an animal cell, a mammalian cell, a rodent cell, a primate cell, a human cell, or a primary cell. In some embodiments, the cell is a primary cell. In some embodiments, the primary cell is a T cell. In some embodiments, the primary cell is a hematopoietic stem cell (HSC). In some embodiments, delivering the engineered transposase system to the target nucleic acid locus comprises delivering an nucleic acid disclosed herein or any vector disclosed herein. In some embodiments, delivering the engineered transposase system to the target nucleic acid locus comprises delivering a nucleic acid comprising an open reading frame encoding the transposase. In some embodiments, the nucleic acid comprises a promoter to which the open reading frame encoding the transposase is operably linked. In some embodiments, delivering the engineered transposase system to the target nucleic acid locus comprises delivering a capped mRNA containing the open reading frame encoding the transposase. In some embodiments, delivering the engineered transposase system to the target nucleic acid locus comprises delivering a translated polypeptide. In some embodiments, the transposase induces a single-stranded break or a double-stranded break at or proximal to the target nucleic acid locus. In some embodiments, the transposase induces a staggered single stranded break within or 5′ to the target locus.

In some aspects, the present disclosure provides for a host cell comprising an open reading frame encoding a heterologous transposase having at least 75% sequence identity to any one of SEQ ID NOs: 1-349 or a variant thereof. In some embodiments, the transposase has at least 75% sequence identity to any one of SEQ ID NOs: 1, 3, 5, 7, 9, 11, 13, 15, or 18-19. In some embodiments, the transposase has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity to any one of SEQ ID NOs: 1, 3, 5, 7, 9, 11, 13, 15, or 18-19. In some embodiments, the transposase has at least 75% sequence identity to any one of SEQ ID NOs: 2, 4, 6, 8, 10, 12, 14, or 17. In some embodiments, the host cell is an E. coli cell. In some embodiments, the E. coli cell is a λDE3 lysogen or the E. coli cell is a BL21(DE3) strain. In some embodiments, the E. coli cell has an ompT lon genotype. In some embodiments, the open reading frame is operably linked to a T7 promoter sequence, a T7-lac promoter sequence, a lac promoter sequence, a tac promoter sequence, a trc promoter sequence, a ParaBAD promoter sequence, a PrhaBAD promoter sequence, a T5 promoter sequence, a cspA promoter sequence, an araPBAD promoter, a strong leftward promoter from phage lambda (pL promoter), or any combination thereof. In some embodiments, the open reading frame comprises a sequence encoding an affinity tag linked in-frame to a sequence encoding the transposase. In some embodiments, the affinity tag is an immobilized metal affinity chromatography (IMAC) tag. In some embodiments, the IMAC tag is a polyhistidine tag. In some embodiments, the affinity tag is a myc tag, a human influenza hemagglutinin (HA) tag, a maltose binding protein (MBP) tag, a glutathione S-transferase (GST) tag, a streptavidin tag, a FLAG tag, or any combination thereof. In some embodiments, the affinity tag is linked in-frame to the sequence encoding the transposase via a linker sequence encoding a protease cleavage site. In some embodiments, the protease cleavage site is a tobacco etch virus (TEV) protease cleavage site, a PreScission® protease cleavage site, a Thrombin cleavage site, a Factor Xa cleavage site, an enterokinase cleavage site, or any combination thereof. In some embodiments, the open reading frame is codon-optimized for expression in the host cell. In some embodiments, the open reading frame is provided on a vector. In some embodiments, the open reading frame is integrated into a genome of the host cell.

In some aspects, the present disclosure provides for a culture comprising any host cell disclosed herein in compatible liquid medium.

In some aspects, the present disclosure provides for a method of producing a transposase, comprising cultivating any host cell disclosed herein in compatible growth medium.

In some embodiments, the method further comprises inducing expression of the transposase by addition of an additional chemical agent or an increased amount of a nutrient. In some embodiments, the additional chemical agent or increased amount of a nutrient comprises Isopropyl β-D-1-thiogalactopyranoside (IPTG) or additional amounts of lactose. In some embodiments, the method further comprises isolating the host cell after the cultivation and lysing the host cell to produce a protein extract. In some embodiments, the method further comprises subjecting the protein extract to IMAC, or ion-affinity chromatography. In some embodiments, the open reading frame comprises a sequence encoding an IMAC affinity tag linked in-frame to a sequence encoding the transposase. In some embodiments, the IMAC affinity tag is linked in-frame to the sequence encoding the transposase via a linker sequence encoding protease cleavage site. In some embodiments, the protease cleavage site comprises a tobacco etch virus (TEV) protease cleavage site, a PreScission® protease cleavage site, a Thrombin cleavage site, a Factor Xa cleavage site, an enterokinase cleavage site, or any combination thereof. In some embodiments, the method further comprises cleaving the IMAC affinity tag by contacting a protease corresponding to the protease cleavage site to the transposase. In some embodiments, the method further comprises performing subtractive IMAC affinity chromatography to remove the affinity tag from a composition comprising the transposase.

In some aspects, the present disclosure provides for a method of disrupting a locus in a cell, comprising contacting to the cell a composition comprising: a double-stranded nucleic acid comprising a cargo nucleotide sequence, wherein the cargo nucleotide sequence is configured to interact with a transposase; and a transposase, wherein: the transposase is configured to transpose the cargo nucleotide sequence to a target nucleic acid locus; the transposase comprises a sequence having at least 75% sequence identity to any one of SEQ ID NOs: 1-349; and the transposase has at least equivalent transposition activity to TnpA transposase in a cell.

In some embodiments, the transposition activity is measured in vitro by introducing the transposase to cells comprising the target nucleic acid locus and detecting transposition of the target nucleic acid locus in the cells. In some embodiments, the composition comprises 20 picomoles (pmol) or less of the transposase. In some embodiments, the composition comprises 1 pmol or less of the transposase.

In some aspects, the present disclosure provides for an engineered transposase system, comprising: a double-stranded nucleic acid comprising a cargo nucleotide sequence, wherein the cargo nucleotide sequence is configured to interact with a transposase; and a transposase, wherein the transposase is configured to transpose the cargo nucleotide sequence to a target nucleic acid locus; and the double-stranded nucleic acid comprises a flanking sequence flanking the cargo sequence, wherein the flanking sequence has at least about 70% sequence identity to at least 90 consecutive nucleotides of any one of SEQ ID NOs: 350-454.

In some embodiments, the transposase is derived from an uncultivated organism. In some embodiments, the transposase is not a TnpA transposase or a TnpB transposase. In some embodiments, the transposase has less than 80% sequence identity to a TnpA transposase. In some embodiments, the transposase has less than 80% sequence identity to a TnpB transposase. In some embodiments, the transposase comprises a sequence having at least 75% sequence identity to any one of SEQ ID NOs: 1-349. In some embodiments, the transposase has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity to any one of SEQ ID NOs: 1, 3, 5, 7, 9, 11, 13, 15, and 18-19. In some embodiments, the transposase comprises a catalytic tyrosine residue. In some embodiments, the transposase is configured to bind a left-hand region comprising a subterminal palindromic sequence and a right-hand region comprising a subterminal palindromic sequence. In some embodiments, the double-stranded deoxyribonucleic acid polynucleotide is transposed as a single-stranded deoxyribonucleic acid polynucleotide. In some embodiments, the transposase comprises one or more nuclear localization signals (NLSs) proximal to an N- or C-terminus of the transposase. In some embodiments, a NLS of the one or more NLSs comprises a sequence at least 80% identical to a sequence from the group consisting of SEQ ID NOs: 455-470. In some embodiments, the double-stranded deoxyribonucleic acid polynucleotide is a eukaryotic, plant, fungal, mammalian, rodent, or human double-stranded deoxyribonucleic acid polynucleotide. In some embodiments, the flanking sequence has at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity to at least 90 consecutive nucleotides of any one of SEQ ID NOs: 350, 352, 355, 356, 359, 361, 362, and 367. In some embodiments, the double-stranded nucleic acid comprises another flanking sequence flanking the cargo sequence, wherein the another flanking sequence has at least about 70% sequence identity to at least 90 consecutive nucleotides of any one of SEQ ID NOs: 350-454. In some embodiments, the another flanking sequence has at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity to at least 90 consecutive nucleotides of any one of SEQ ID NOs: 351, 353, 354, 357, 358, 360, 363, and 366. In some embodiments, the flanking sequence flanks a left end of the cargo nucleic acid sequence and wherein the another flanking sequence flanks a right end of the cargo nucleic acid sequence. In some embodiments, the transposase is configured to recognize an insertion motif adjacent to the target nucleic acid locus. In some embodiments, the insertion motif comprises at least three, four, five, or six consecutive nucleotides of the sequence AATGAC.

In some aspects, the present disclosure provides for a deoxyribonucleic acid polynucleotide encoding any engineered transposase system disclosed herein.

In some aspects, the present disclosure provides for a method for binding, nicking, cleaving, marking, modifying, or transposing a double-stranded deoxyribonucleic acid polynucleotide comprising a cargo sequence, the method comprising: contacting the double-stranded deoxyribonucleic acid polynucleotide with a transposase configured to transpose the cargo nucleotide sequence to a target nucleic acid locus; wherein the double-stranded deoxyribonucleic acid polynucleotide comprises a flanking sequence flanking the cargo sequence, wherein the flanking sequence has at least about 70% sequence identity to at least 90 consecutive nucleotides of any one of SEQ ID NOs: 350-454.

In some embodiments, the transposase is derived from an uncultivated organism. In some embodiments, the transposase is not a TnpA transposase or a TnpB transposase. In some embodiments, the transposase has less than 80% sequence identity to a TnpA transposase In some embodiments, the transposase has less than 80% sequence identity to a TnpB transposase. In some embodiments, the transposase comprises a sequence having at least 75% sequence identity to any one of SEQ ID NOs: 1-349. In some embodiments, the transposase has at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity to any one of SEQ ID NOs: 1, 3, 5, 7, 9, 11, 13, 15, and 18-19. In some embodiments, the transposase comprises a catalytic tyrosine residue. In some embodiments, the transposase is configured to bind a left-hand region comprising a subterminal palindromic sequence and a right-hand region comprising a subterminal palindromic sequence. In some embodiments, the transposase is compatible with a left-hand recognition sequence or a right-hand recognition sequence. In some embodiments, the double-stranded deoxyribonucleic acid polynucleotide is transposed as a single-stranded deoxyribonucleic acid polynucleotide. In some embodiments, the transposase comprises one or more nuclear localization signals (NLSs) proximal to an N- or C-terminus of the transposase. In some embodiments, a NLS of the one or more NLSs comprises a sequence at least 80% identical to a sequence from the group consisting of SEQ ID NOs: 455-470. In some embodiments, the double-stranded deoxyribonucleic acid polynucleotide is a eukaryotic, plant, fungal, mammalian, rodent, or human double-stranded deoxyribonucleic acid polynucleotide. In some embodiments, the flanking sequence has at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity to at least 90 consecutive nucleotides of any one of SEQ ID NOs: 350, 352, 355, 356, 359, 361, 362, and 367. In some embodiments, the double-stranded deoxyribonucleic acid polynucleotide comprises another flanking sequence flanking the cargo sequence, wherein the another flanking sequence has at least about 70% sequence identity to at least 90 consecutive nucleotides of any one of SEQ ID NOs: 350-454. In some embodiments, the another flanking sequence has at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity to at least 90 consecutive nucleotides of any one of SEQ ID NOs: 351, 353, 354, 357, 358, 360, 363, and 366. In some embodiments, the flanking sequence flanks a left end of the cargo nucleic acid sequence and wherein the another flanking sequence flanks a right end of the cargo nucleic acid sequence. In some embodiments, the transposase is configured to recognize an insertion motif adjacent to the target nucleic acid locus. In some embodiments, the insertion motif comprises at least three, four, five, or six consecutive nucleotides of the sequence AATGAC.

In some aspects, the present disclosure provides for a method of modifying a target nucleic acid locus, the method comprising delivering to the target nucleic acid locus an engineered transposase system disclosed herein, wherein the transposase is configured to transpose the cargo nucleotide sequence to the target nucleic acid locus, and wherein the complex is configured such that upon binding of the complex to the target nucleic acid locus, the complex modifies the target nucleic acid locus.

In some embodiments, modifying the target nucleic acid locus comprises binding, nicking, cleaving, marking, modifying, or transposing the target nucleic acid locus. In some embodiments, the target nucleic acid locus comprises deoxyribonucleic acid (DNA). In some embodiments, the target nucleic acid locus comprises genomic DNA, viral DNA, or bacterial DNA. In some embodiments, the target nucleic acid locus is in vitro. In some embodiments, the target nucleic acid locus is within a cell. In some embodiments, the cell is a prokaryotic cell, a bacterial cell, a eukaryotic cell, a fungal cell, a plant cell, an animal cell, a mammalian cell, a rodent cell, a primate cell, a human cell, or a primary cell. In some embodiments, the cell is a primary cell. In some embodiments, the primary cell is a T cell. In some embodiments, the primary cell is a hematopoietic stem cell (HSC). In some embodiments, delivering the engineered transposase system to the target nucleic acid locus comprises delivering a nucleic acid comprising an open reading frame encoding the transposase. In some embodiments, the nucleic acid comprises a promoter to which the open reading frame encoding the transposase is operably linked. In some embodiments, delivering the engineered transposase system to the target nucleic acid locus comprises delivering a capped mRNA containing the open reading frame encoding the transposase. In some embodiments, delivering the engineered transposase system to the target nucleic acid locus comprises delivering a translated polypeptide. In some embodiments, the transposase induces a single-stranded break or a double-stranded break at or proximal to the target nucleic acid locus. In some embodiments, the transposase induces a staggered single stranded break within or 5′ to the target locus.

In some aspects, the present disclosure provides for an engineered transposase system, comprising: (a) a double-stranded nucleic acid comprising a cargo nucleotide sequence, wherein the cargo nucleotide sequence is configured to interact with a transposase; and (b) a transposase, wherein: (i) the transposase is configured to transpose the cargo nucleotide sequence to a target nucleic acid locus; and (ii) the transposase is derived from an uncultivated microorganism. In some embodiments, the cargo nucleotide sequence is a heterologous sequence. In some embodiments, the cargo nucleotide sequence is an engineered sequence. In some embodiments, the cargo nucleotide sequence is not a wild-type genome sequence present in an organism In some embodiments, the transposase comprises a sequence having at least 75% sequence identity to any one of SEQ ID NOs: 1-349. In some embodiments, the transposase is not a TnpA transposase or a TnpB transposase. In some embodiments, the transposase has less than 80% sequence identity to a TnpA transposase. In some embodiments, the transposase has less than 80% sequence identity to a TnpB transposase. In some embodiments, the transposase comprises a catalytic tyrosine residue. In some embodiments, the transposase is configured to bind a left-hand region comprising a subterminal palindromic sequence and a right-hand region comprising a subterminal palindromic sequence. In some embodiments, the transposase is configured to transpose the cargo nucleotide sequence as single-stranded deoxyribonucleic acid polynucleotide.

In some embodiments, the transposase comprises one or more nuclear localization sequences (NLSs) proximal to an N- or C-terminus of the transposase. In some embodiments, the NLS comprises a sequence at least 80% identical to a sequence from the group consisting of SEQ ID NO: 455-470. In some embodiments, the sequence identity is determined by a BLASTP, CLUSTALW, MUSCLE, MAFFT, or CLUSTALW with the parameters of the Smith-Waterman homology search algorithm. In some embodiments, the sequence identity is determined by the BLASTP homology search algorithm using parameters of a wordlength (W) of 3, an expectation (E) of 10, and a BLOSUM62 scoring matrix setting gap costs at existence of 11, extension of 1, and using a conditional compositional score matrix adjustment.

In some aspects, the present disclosure provides for an engineered transposase system, comprising: (a) a double-stranded nucleic acid comprising a cargo nucleotide sequence, wherein the cargo nucleotide sequence is configured to interact with a transposase; and (b) a transposase, wherein: (i) the transposase is configured to transpose the cargo nucleotide sequence to a target nucleic acid locus; and (ii) the transposase comprises a sequence having at least 75% sequence identity to any one of SEQ ID NOs: 1-349. In some embodiments, the transposase is derived from an uncultivated microorganism. In some embodiments, the transposase is not a TnpA transposase or a TnpB transposase. In some embodiments, the transposase has less than 80% sequence identity to a TnpA transposase. In some embodiments, the transposase has less than 80% sequence identity to a TnpB transposase. In some embodiments, the transposase comprises a catalytic tyrosine residue. In some embodiments, the transposase is configured to bind a left-hand region comprising a subterminal palindromic sequence and a right-hand region comprising a subterminal palindromic sequence. In some embodiments, the transposase is configured to transpose the cargo nucleotide sequence as single-stranded deoxyribonucleic acid polynucleotide.

In some embodiments, the sequence identity is determined by a BLASTP, CLUSTALW, MUSCLE, MAFFT, or CLUSTALW with the parameters of the Smith-Waterman homology search algorithm. In some embodiments, the sequence identity is determined by the BLASTP homology search algorithm using parameters of a wordlength (W) of 3, an expectation (E) of 10, and a BLOSUM62 scoring matrix setting gap costs at existence of 11, extension of 1, and using a conditional compositional score matrix adjustment.

In some aspects, the present disclosure provides for a deoxyribonucleic acid polynucleotide encoding the engineered transposase system of any one of the aspects or embodiments described herein

In some aspects, the present disclosure provides for a nucleic acid comprising an engineered nucleic acid sequence optimized for expression in an organism, wherein the nucleic acid encodes a transposase, and wherein the transposase is derived from an uncultivated microorganism, wherein the organism is not the uncultivated microorganism. In some embodiments, the transposase comprises a variant having at least 75% sequence identity to any one of SEQ ID NOs: 1-349. In some embodiments, the transposase comprises a sequence encoding one or more nuclear localization sequences (NLSs) proximal to an N- or C-terminus of the transposase. In some embodiments, the NLS comprises a sequence selected from SEQ ID NOs: 455-470. In some embodiments, the NLS comprises SEQ ID NO: 456. In some embodiments, the NLS is proximal to the N-terminus of the transposase. In some embodiments, the NLS comprises SEQ ID NO: 455. In some embodiments, the NLS is proximal to the C-terminus of the transposase. In some embodiments, the organism is prokaryotic, bacterial, eukaryotic, fungal, plant, mammalian, rodent, or human.

In some aspects, the present disclosure provides for a vector comprising the nucleic acid of any one of the aspects or embodiments described herein. In some embodiments, the vector further comprises a nucleic acid encoding a cargo nucleotide sequence configured to form a complex with the transposase. In some embodiments, the vector is a plasmid, a minicircle, a CELiD, an adeno-associated virus (AAV) derived virion, or a lentivirus.

In some aspects, the present disclosure provides for a cell comprising the vector of any one of any one of the aspects or embodiments described herein.

In some aspects, the present disclosure provides for a method of manufacturing a transposase, comprising cultivating the cell of any one of the aspects or embodiments described herein.

In some aspects, the present disclosure provides for a method for binding, nicking, cleaving, marking, modifying, or transposing a double-stranded deoxyribonucleic acid polynucleotide, comprising: (a) contacting the double-stranded deoxyribonucleic acid polynucleotide with a transposase configured to transpose the cargo nucleotide sequence to a target nucleic acid locus; wherein the transposase comprises a sequence having at least 75% sequence identity to any one of SEQ ID NOs: 1-349. In some embodiments, the transposase is derived from an uncultivated microorganism. In some embodiments, the transposase is not a TnpA transposase or a TnpB transposase. In some embodiments, the transposase has less than 80% sequence identity to a TnpA transposase. In some embodiments, the transposase has less than 80% sequence identity to a TnpB transposase. In some embodiments, the transposase comprises a catalytic tyrosine residue. In some embodiments, the transposase is configured to bind a left-hand region comprising a subterminal palindromic sequence and a right-hand region comprising a subterminal palindromic sequence. In some embodiments, the double-stranded deoxyribonucleic acid polynucleotide is transposed as a single-stranded deoxyribonucleic acid polynucleotide. In some embodiments, the double-stranded deoxyribonucleic acid polynucleotide is a eukaryotic, plant, fungal, mammalian, rodent, or human double-stranded deoxyribonucleic acid polynucleotide.

In some aspects, the present disclosure provides for a method of modifying a target nucleic acid locus, the method comprising delivering to the target nucleic acid locus the engineered transposase system of any one of the aspects or embodiments described herein, wherein the transposase is configured to transpose the cargo nucleotide sequence to the target nucleic acid locus, and wherein the complex is configured such that upon binding of the complex to the target nucleic acid locus, the complex modifies the target nucleic acid locus. In some embodiments, modifying the target nucleic acid locus comprises binding, nicking, cleaving, marking, modifying, or transposing the target nucleic acid locus. In some embodiments, the target nucleic acid locus comprises deoxyribonucleic acid (DNA). In some embodiments, the target nucleic acid locus comprises genomic DNA, viral DNA, or bacterial DNA. In some embodiments, the target nucleic acid locus is in vitro. In some embodiments, the target nucleic acid locus is within a cell. In some embodiments, the cell is a prokaryotic cell, a bacterial cell, a eukaryotic cell, a fungal cell, a plant cell, an animal cell, a mammalian cell, a rodent cell, a primate cell, a human cell, or a primary cell. In some embodiments, the cell is a primary cell. In some embodiments, the primary cell is a T cell. In some embodiments, the primary cell is a hematopoietic stem cell (HSC). In some embodiments, delivering the engineered transposase system to the target nucleic acid locus comprises delivering the nucleic acid of any one of the aspects or embodiments described herein or the vector of any of the aspects or embodiments described herein. In some embodiments, delivering the engineered transposase system to the target nucleic acid locus comprises delivering a nucleic acid comprising an open reading frame encoding the transposase. In some embodiments, the nucleic acid comprises a promoter to which the open reading frame encoding the transposase is operably linked. In some embodiments, delivering the engineered transposase system to the target nucleic acid locus comprises delivering a capped mRNA containing the open reading frame encoding the transposase. In some embodiments, delivering the engineered transposase system to the target nucleic acid locus comprises delivering a translated polypeptide. In some embodiments, the transposase induces a single-stranded break or a double-stranded break at or proximal to the target nucleic acid locus. In some embodiments, the transposase induces a staggered single stranded break within or 5′ to the target locus.

In some aspects, the present disclosure provides for a host cell comprising an open reading frame encoding a heterologous transposase having at least 75% sequence identity to any one of SEQ ID NOs: 1-349 or a variant thereof. In some embodiments, the transposase has at least 75% sequence identity to any one of SEQ ID NOs: 1, 3, 5, 7, 9, 11, 13, 15, or 16. In some embodiments, the transposase has at least 75% sequence identity to any one of SEQ ID NOs: 2, 4, 6, 8, 10, 12, 14, or 17. In some embodiments, the host cell is an E. coli cell. In some embodiments, the E. coli cell is a λDE3 lysogen or the E. coli cell is a BL21(DE3) strain. In some embodiments, the E. coli cell has an ompT ion genotype. In some embodiments, the open reading frame is operably linked to a T7 promoter sequence, a T7-lac promoter sequence, a lac promoter sequence, a tac promoter sequence, a trc promoter sequence, a ParaBAD promoter sequence, a PrhaBAD promoter sequence, a T5 promoter sequence, a cspA promoter sequence, an araPBAD promoter, a strong leftward promoter from phage lambda (pL promoter), or any combination thereof. In some embodiments, the open reading frame comprises a sequence encoding an affinity tag linked in-frame to a sequence encoding the transposase. In some embodiments, the affinity tag is an immobilized metal affinity chromatography (IMAC) tag. In some embodiments, the IMAC tag is a polyhistidine tag. In some embodiments, the affinity tag is a myc tag, a human influenza hemagglutinin (HA) tag, a maltose binding protein (MBP) tag, a glutathione S-transferase (GST) tag, a streptavidin tag, a FLAG tag, or any combination thereof. In some embodiments, the affinity tag is linked in-frame to the sequence encoding the transposase via a linker sequence encoding a protease cleavage site. In some embodiments, the protease cleavage site is a tobacco etch virus (TEV) protease cleavage site, a PreScission® protease cleavage site, a Thrombin cleavage site, a Factor Xa cleavage site, an enterokinase cleavage site, or any combination thereof. In some embodiments, the open reading frame is codon-optimized for expression in the host cell. In some embodiments, the open reading frame is provided on a vector. In some embodiments, the open reading frame is integrated into a genome of the host cell.

In some aspects, the present disclosure provides for a culture comprising the host cell of any one of the aspects or embodiments described herein in compatible liquid medium.

In some aspects, the present disclosure provides for a method of producing a transposase, comprising cultivating the host cell of any one of the aspects or embodiments described herein in compatible growth medium. In some embodiments, the method further comprises inducing expression of the transposase by addition of an additional chemical agent or an increased amount of a nutrient. In some embodiments, the additional chemical agent or increased amount of a nutrient comprises Isopropyl β-D-1-thiogalactopyranoside (IPTG) or additional amounts of lactose. In some embodiments, the method further comprises isolating the host cell after the cultivation and lysing the host cell to produce a protein extract. In some embodiments, the method further comprises subjecting the protein extract to IMAC, or ion-affinity chromatography. In some embodiments, the open reading frame comprises a sequence encoding an IMAC affinity tag linked in-frame to a sequence encoding the transposase. In some embodiments, the IMAC affinity tag is linked in-frame to the sequence encoding the transposase via a linker sequence encoding protease cleavage site. In some embodiments, the protease cleavage site comprises a tobacco etch virus (TEV) protease cleavage site, a PreScission® protease cleavage site, a Thrombin cleavage site, a Factor Xa cleavage site, an enterokinase cleavage site, or any combination thereof. In some embodiments, the method further comprises cleaving the IMAC affinity tag by contacting a protease corresponding to the protease cleavage site to the transposase. In some embodiments, the method further comprises performing subtractive IMAC affinity chromatography to remove the affinity tag from a composition comprising the transposase.

In some aspects, the present disclosure provides for a method of disrupting a locus in a cell, comprising contacting to the cell a composition comprising: (a) a double-stranded nucleic acid comprising a cargo nucleotide sequence, wherein the cargo nucleotide sequence is configured to interact with a transposase; and (b) a transposase, wherein: (i) the transposase is configured to transpose the cargo nucleotide sequence to a target nucleic acid locus; (ii) the transposase comprises a sequence having at least 75% sequence identity to any one of SEQ ID NOs: 1-349; and (iii) the transposase has at least equivalent transposition activity to TnpA transposase in a cell. In some embodiments, the transposition activity is measured in vitro by introducing the transposase to cells comprising the target nucleic acid locus and detecting transposition of the target nucleic acid locus in the cells. In some embodiments, the composition comprises 20 pmoles or less of the transposase. In some embodiments, the composition comprises 1 pmol or less of the transposase.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIGS. 1A and 1B depict MG transposases. FIG. 1A depicts the organization of a transposon comprising the tyrosine (Y1) transposase MG92-1 locus. MG92-1 is encoded at the 5′ end of the transposon, followed by the accessory transposition protein TnpB and other cargo. The transposon ends contain direct repeats of 16-17 bp, and they exhibit secondary structure likely involved in transposition activity. FIG. 1B depicts multiple sequence alignment of MG Y1 transposase homologs. Catalytic residues HUH and Y are highlighted on the consensus sequence and on the MSA (boxes).

FIG. 2 depicts a phylogenetic tree of TnpA protein sequences. The tree was built from a multiple sequence alignment of 414 novel TnpA sequences recovered here (black dots) and 19 reference TnpA sequences (grey dots). Labels for references sequences were included.

FIG. 3 depicts an example insertion sequence IS200/IS605 MG92-28. Top panel: Genomic context of the MG92-28 insertion sequence encoding the TnpA-like transposase and its associated TnpB-like gene. Both genes are flanked by LE and RE (boxes) predicted from covariance models. Bottom panel: LE (top left) and RE (bottom right) delineate the boundaries of the insertion sequence. Region predicted by the covariance models is annotated as arrows below the sequence. LE and RE secondary structures are shown for each end.

FIG. 4 depicts a Western blot of TnpA-like proteins expressed in PureExpress. Lanes are: ladder, 1: HpTnpA, 2: HhTpA, 3: 92-2, 4: 92-3, 5: 92-4, 6: 92-5, 7: 92-6, 8: 92-7, 9: 92-8, 10: 92-10, 11: 92-11. HpTnpA and HhTpA are positive controls from H. pylori and H. Heilmannii, respectively. Molecular weights range from 17-23 kilodaltons (kDa).

FIG. 5A depicts the PCR product for the LE of the transposition reaction. All reactions have the protein and its paired specific cargo, except the control lane where the cargo is specified. Lanes are: 1: Ladder, 2: negative control NTC with HpTnpA cargo, 3: 92-1, 4: 92-2, 5: 92-3, 6: 92-4, 7: 92-5, 8: 92-6, 9: 92-7, 10: 92-8, 11: 92-10, 12: 92-11, 13: HpTnpA, 14; HhTnpA. Expected transposition product can range from 200 to 300 bp depending on LE size and is marked with an arrow. The band at <200 bp in 92-5 is related to non-specific primer interactions. FIG. 5B depicts the PCR product for the RE of the transposition reaction. All reactions have the protein and its paired specific cargo, except the control lane where the cargo is specified. Lanes are: 1: NTC with HpTnpA cargo, 2: 92-1, 3: 92-2, 4: 92-3, 5: 92-4, 6: 92-5, 7: 92-6, 8: 92-7, 9: 92-8, 10: 92-10, 11: 92-11, 12: HpTnpA, 13; HhTnpA, and 14: ladder. Expected transposition product can range from 300 to 500 bp depending on RE size and is marked with an arrow. Transposition that occurs into the 8N region will have a much weaker band than transposition into flanking sequence, so the faint bands are expected.

FIG. 6 depicts Sanger sequencing data confirming transposition for MG92-3. The chromatogram trace is shown mapped to the cargo sequence, where shaded letters match the cargo. At the cleavage point (arrow) the trace instead maps onto the target sequence (boxed). Analysis of the target reveals the insertion motif, which is shared sequence between the LE and the target. Downstream hairpins with flanking non-canonical base interactions can be identified.

FIG. 7 depicts Sanger sequencing data confirming transposition for MG92-3. The chromatogram trace is shown mapped to the cargo, and shaded letters match the cargo. At the cleavage point (arrow) the trace instead maps onto the target sequence (boxed). Analysis of the target reveals the insertion motif. The cleavage position in the putative RE defines the boundary of the RE, which folds into a canonical hairpin to allow TnpA recognition and strand cleavage (inset of dotted box).

FIG. 8 depicts analysis of chimeric NGS reads showing cargo and target sequence joints which were analyzed to determine the breakpoint. The x-axis is the position along the cargo sequence and the y-axis is the count of reads which transition at that position. The identified peak in the breakpoint at 2030 nt on the cargo matches the breakpoint identified in Sanger sequencing, confirming the position of LE cleavage.

FIG. 9 depicts NGS sequencing data confirming transposition for MG92-4. The NGS reads are shown mapped to the target, and light-shaded letters match the cargo. At the cleavage point (arrow) the trace instead maps onto the cargo sequence (boxed). The cleavage position in the putative RE defines the boundary of the RE, which folds into a canonical hairpin to allow TnpA recognition and strand cleavage (inset of dotted box). The NGS read histogram shows the frequency of reads corresponding to this breakpoint on the cargo.

### BRIEF DESCRIPTION OF THE SEQUENCE LISTING

The Sequence Listing filed herewith provides exemplary polynucleotide and polypeptide sequences for use in methods, compositions, and systems according to the disclosure. Below are exemplary descriptions of sequences therein.

### MG92

SEQ ID NOs: 1-349 show the full-length peptide sequences of MG92 transposition proteins.

SEQ ID NOs: 350-454 show the full-length peptide sequences of MG92 transposon ends.

### Nuclear Localization Sequences

SEQ ID NOs: 455-470 show the full-length peptide sequences of nuclear localization sequences (NLSs) suitable for use with MG92 transposition proteins described herein.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The practice of some methods disclosed herein employ, unless otherwise indicated, techniques of immunology, biochemistry, chemistry, molecular biology, microbiology, cell biology, genomics, and recombinant DNA. See for example Sambrook and Green, Molecular Cloning: A Laboratory Manual, 4th Edition (2012); the series Current Protocols in Molecular Biology (F. M. Ausubel, et al. eds.); the series Methods In Enzymology (Academic Press, Inc.), PCR 2: A Practical Approach (M. J. MacPherson, B. D. Hames and G. R. Taylor eds. (1995)), Harlow and Lane, eds. (1988) Antibodies, A Laboratory Manual, and Culture of Animal Cells: A Manual of Basic Technique and Specialized Applications, 6th Edition (R. I. Freshney, ed. (2010)) (which is entirely incorporated by reference herein).

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”.

The term “about” or “approximately” means within an acceptable error range for the particular value as determined by one of ordinary skill in the art, which will depend in part on how the value is measured or determined, i.e., the limitations of the measurement system. For example, “about” can mean within one or more than one standard deviation, per the practice in the art. Alternatively, “about” can mean a range of up to 20%, up to 15%, up to 10%, up to 5%, or up to 1% of a given value.

As used herein, a “cell” generally refers to a biological cell. A cell may be the basic structural, functional and/or biological unit of a living organism. A cell may originate from any organism having one or more cells. Some non-limiting examples include: a prokaryotic cell, eukaryotic cell, a bacterial cell, an archaeal cell, a cell of a single-cell eukaryotic organism, a protozoa cell, a cell from a plant (e.g., cells from plant crops, fruits, vegetables, grains, soy bean, corn, maize, wheat, seeds, tomatoes, rice, cassava, sugarcane, pumpkin, hay, potatoes, cotton, cannabis, tobacco, flowering plants, conifers, gymnosperms, fems, clubmosses, hornworts, liverworts, mosses), an algal cell, (e.g., Botryococcus braunii, Chlamydomonas reinhardtii, Nannochloropsis gaditana, Chlorella pyrenoidosa, Sargassum patens C. Agardh, and the like), seaweeds (e.g., kelp), a fungal cell (e.g., a yeast cell, a cell from a mushroom), an animal cell, a cell from an invertebrate animal (e.g., fruit fly, cnidarian, echinoderm, nematode, etc.), a cell from a vertebrate animal (e.g., fish, amphibian, reptile, bird, mammal), a cell from a mammal (e.g., a pig, a cow, a goat, a sheep, a rodent, a rat, a mouse, a non-human primate, a human, etc.), and etcetera. Sometimes a cell is not originating from a natural organism (e.g., a cell can be a synthetically made, sometimes termed an artificial cell).

The term “nucleotide,” as used herein, generally refers to a base-sugar-phosphate combination. A nucleotide may comprise a synthetic nucleotide. A nucleotide may comprise a synthetic nucleotide analog. Nucleotides may be monomeric units of a nucleic acid sequence (e.g., deoxyribonucleic acid (DNA) and ribonucleic acid (RNA)). The term nucleotide may include ribonucleoside triphosphates adenosine triphosphate (ATP), uridine triphosphate (UTP), cytosine triphosphate (CTP), guanosine triphosphate (GTP) and deoxyribonucleoside triphosphates such as dATP, dCTP, dITP, dUTP, dGTP, dTTP, or derivatives thereof. Such derivatives may include, for example, [aS]dATP, 7-deaza-dGTP and 7-deaza-dATP, and nucleotide derivatives that confer nuclease resistance on the nucleic acid molecule containing them. The term nucleotide as used herein may refer to dideoxyribonucleoside triphosphates (ddNTPs) and their derivatives. Illustrative examples of dideoxyribonucleoside triphosphates may include, but are not limited to, ddATP, ddCTP, ddGTP, ddITP, and ddTTP. A nucleotide may be unlabeled or detectably labeled, such as using moieties comprising optically detectable moieties (e.g., fluorophores). Labeling may also be carried out with quantum dots. Detectable labels may include, for example, radioactive isotopes, fluorescent labels, chemiluminescent labels, bioluminescent labels, and enzyme labels. Fluorescent labels of nucleotides may include but are not limited fluorescein, 5-carboxyfluorescein (FAM), 2′7′-dimethoxy-4′5-dichloro-6-carboxyfluorescein (JOE), rhodamine, 6-carboxyrhodamine (R6G), N,N,N′,N′-tetramethyl-6-carboxyrhodamine (TAMRA), 6-carboxy-X-rhodamine (ROX), 4-(4′dimethylaminophenylazo) benzoic acid (DABCYL), Cascade Blue, Oregon Green, Texas Red, Cyanine and 5-(2′-aminoethyl)aminonaphthalene-1-sulfonic acid (EDANS). Specific examples of fluorescently labeled nucleotides can include [R6G]dUTP, [TAMRA]dUTP, [RI 10]dCTP, [R6G]dCTP, [TAMRA]dCTP, [JOE]ddATP, [R6G]ddATP, [FAM]ddCTP, [R110]ddCTP, [TAMRA]ddGTP, [ROX]ddTTP, [dR6G]ddATP, [dR110]ddCTP, [dTAMRA]ddGTP, and [dROX]ddTTP available from Perkin Elmer, Foster City, Calif; FluoroLink DeoxyNucleotides, FluoroLink Cy3-dCTP, FluoroLink Cy5-dCTP, FluoroLink Fluor X-dCTP, FluoroLink Cy3-dUTP, and FluoroLink Cy5-dUTP available from Amersham, Arlington Heights, Il.; Fluorescein-15-dATP, Fluorescein-12-dUTP, Tetramethyl-rodamine-6-dUTP, IR770-9-dATP, Fluorescein-12-ddUTP, Fluorescein-12-UTP, and Fluorescein-15-2′-dATP available from Boehringer Mannheim, Indianapolis, Ind.; and Chromosome Labeled Nucleotides, BODIPY-FL-14-UTP, BODIPY-FL-4-UTP, BODIPY-TMR-14-UTP, BODIPY-TMR-14-dUTP, BODIPY-TR-14-UTP, BODIPY-TR-14-dUTP, Cascade Blue-7-UTP, Cascade Blue-7-dUTP, fluorescein-12-UTP, fluorescein-12-dUTP, Oregon Green 488-5-dUTP, Rhodamine Green-5-UTP, Rhodamine Green-5-dUTP, tetramethylrhodamine-6-UTP, tetramethylrhodamine-6-dUTP, Texas Red-5-UTP, Texas Red-5-dUTP, and Texas Red-12-dUTP available from Molecular Probes, Eugene, Oreg. Nucleotides can also be labeled or marked by chemical modification. A chemically-modified single nucleotide can be biotin-dNTP. Some non-limiting examples of biotinylated dNTPs can include, biotin-dATP (e.g., bio-N6-ddATP, biotin-14-dATP), biotin-dCTP (e.g., biotin-11-dCTP, biotin-14-dCTP), and biotin-dUTP (e.g., biotin-11-dUTP, biotin-16-dUTP, biotin-20-dUTP).

The terms “polynucleotide,” “oligonucleotide,” and “nucleic acid” are used interchangeably to generally refer to a polymeric form of nucleotides of any length, either deoxyribonucleotides or ribonucleotides, or analogs thereof, either in single-, double-, or multi-stranded form. A polynucleotide may be exogenous or endogenous to a cell. A polynucleotide may exist in a cell-free environment. A polynucleotide may be a gene or fragment thereof. A polynucleotide may be DNA. A polynucleotide may be RNA. A polynucleotide may have any three-dimensional structure and may perform any function. A polynucleotide may comprise one or more analogs (e.g., altered backbone, sugar, or nucleobase). If present, modifications to the nucleotide structure may be imparted before or after assembly of the polymer. Some non-limiting examples of analogs include: 5-bromouracil, peptide nucleic acid, xeno nucleic acid, morpholinos, locked nucleic acids, glycol nucleic acids, threose nucleic acids, dideoxynucleotides, cordycepin, 7-deaza-GTP, fluorophores (e.g., rhodamine or fluorescein linked to the sugar), thiol-containing nucleotides, biotin-linked nucleotides, fluorescent base analogs, CpG islands, methyl-7-guanosine, methylated nucleotides, inosine, thiouridine, pseudouridine, dihydrouridine, queuosine, and wyosine. Non-limiting examples of polynucleotides include coding or non-coding regions of a gene or gene fragment, loci (locus) defined from linkage analysis, exons, introns, messenger RNA (mRNA), transfer RNA (tRNA), ribosomal RNA (rRNA), short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, cell-free polynucleotides including cell-free DNA (cfDNA) and cell-free RNA (cfRNA), nucleic acid probes, and primers. The sequence of nucleotides may be interrupted by non-nucleotide components.

The terms “transfection” or “transfected” generally refer to introduction of a nucleic acid into a cell by non-viral or viral-based methods. The nucleic acid molecules may be gene sequences encoding complete proteins or functional portions thereof. See, e.g., Sambrook et al., 1989, Molecular Cloning: A Laboratory Manual, 18.1-18.88 (which is entirely incorporated by reference herein).

The terms “peptide,” “polypeptide,” and “protein” are used interchangeably herein to generally refer to a polymer of at least two amino acid residues joined by peptide bond(s). This term does not connote a specific length of polymer, nor is it intended to imply or distinguish whether the peptide is produced using recombinant techniques, chemical or enzymatic synthesis, or is naturally occurring. The terms apply to naturally occurring amino acid polymers as well as amino acid polymers comprising at least one modified amino acid. In some embodiments, the polymer may be interrupted by non-amino acids. The terms include amino acid chains of any length, including full length proteins, and proteins with or without secondary and/or tertiary structure (e.g., domains). The terms also encompass an amino acid polymer that has been modified, for example, by disulfide bond formation, glycosylation, lipidation, acetylation, phosphorylation, oxidation, and any other manipulation such as conjugation with a labeling component. The terms “amino acid” and “amino acids,” as used herein, generally refer to natural and non-natural amino acids, including, but not limited to, modified amino acids and amino acid analogues. Modified amino acids may include natural amino acids and non-natural amino acids, which have been chemically modified to include a group or a chemical moiety not naturally present on the amino acid. Amino acid analogues may refer to amino acid derivatives. The term “amino acid” includes both D-amino acids and L-amino acids.

As used herein, the “non-native” can generally refer to a nucleic acid or polypeptide sequence that is not found in a native nucleic acid or protein. Non-native may refer to affinity tags. Non-native may refer to fusions. Non-native may refer to a naturally occurring nucleic acid or polypeptide sequence that comprises mutations, insertions and/or deletions. A non-native sequence may exhibit and/or encode for an activity (e.g., enzymatic activity, methyltransferase activity, acetyltransferase activity, kinase activity, ubiquitinating activity, etc.) that may also be exhibited by the nucleic acid and/or polypeptide sequence to which the non-native sequence is fused. A non-native nucleic acid or polypeptide sequence may be linked to a naturally-occurring nucleic acid or polypeptide sequence (or a variant thereof) by genetic engineering to generate a chimeric nucleic acid and/or polypeptide sequence encoding a chimeric nucleic acid and/or polypeptide.

The term “promoter”, as used herein, generally refers to the regulatory DNA region which controls transcription or expression of a gene and which may be located adjacent to or overlapping a nucleotide or region of nucleotides at which RNA transcription is initiated. A promoter may contain specific DNA sequences which bind protein factors, often referred to as transcription factors, which facilitate binding of RNA polymerase to the DNA leading to gene transcription. A ‘basal promoter’, also referred to as a ‘core promoter’, may generally refer to a promoter that contains all the basic elements to promote transcriptional expression of an operably linked polynucleotide. In some embodiments eukaryotic basal promoters contain a TATA-box and/or a CAAT box.

The term “expression”, as used herein, generally refers to the process by which a nucleic acid sequence or a polynucleotide is transcribed from a DNA template (such as into mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

As used herein, “operably linked”, “operable linkage”, “operatively linked”, or grammatical equivalents thereof generally refer to juxtaposition of genetic elements, e.g., a promoter, an enhancer, a polyadenylation sequence, etc., wherein the elements are in a relationship permitting them to operate in the expected manner. For instance, a regulatory element, which may comprise promoter and/or enhancer sequences, is operatively linked to a coding region if the regulatory element helps initiate transcription of the coding sequence. There may be intervening residues between the regulatory element and coding region so long as this functional relationship is maintained.

A “vector” as used herein, generally refers to a macromolecule or association of macromolecules that comprises or associates with a polynucleotide and which may be used to mediate delivery of the polynucleotide to a cell. Examples of vectors include plasmids, viral vectors, liposomes, and other gene delivery vehicles. The vector generally comprises genetic elements, e.g., regulatory elements, operatively linked to a gene to facilitate expression of the gene in a target.

As used herein, “an expression cassette” and “a nucleic acid cassette” are used interchangeably generally to refer to a combination of nucleic acid sequences or elements that are expressed together or are operably linked for expression. In some embodiments, an expression cassette refers to the combination of regulatory elements and a gene or genes to which they are operably linked for expression.

A “functional fragment” of a DNA or protein sequence generally refers to a fragment that retains a biological activity (either functional or structural) that is substantially similar to a biological activity of the full-length DNA or protein sequence. A biological activity of a DNA sequence may be its ability to influence expression in a manner attributed to the full-length sequence.

As used herein, an “engineered” object generally indicates that the object has been modified by human intervention. According to non-limiting examples: a nucleic acid may be modified by changing its sequence to a sequence that does not occur in nature; a nucleic acid may be modified by ligating it to a nucleic acid that it does not associate with in nature such that the ligated product possesses a function not present in the original nucleic acid; an engineered nucleic acid may synthesized in vitro with a sequence that does not exist in nature; a protein may be modified by changing its amino acid sequence to a sequence that does not exist in nature; an engineered protein may acquire a new function or property. An “engineered” system comprises at least one engineered component.

As used herein, “synthetic” and “artificial” can generally be used interchangeably to refer to a protein or a domain thereof that has low sequence identity (e.g., less than 50% sequence identity, less than 25% sequence identity, less than 10% sequence identity, less than 5% sequence identity, less than 1% sequence identity) to a naturally occurring human protein. For example, VPR and VP64 domains are synthetic transactivation domains.

As used herein, the term “transposable element” refers to a DNA sequence that can move from one location in the genome to another (i.e., they can be “transposed”). Transposable elements can be generally divided into two classes. Class I transposable elements, or “retrotransposons”, are transposed via transcription and translation of an RNA intermediate which is subsequently reincorporated into its new location into the genome via reverse transcription (a process mediated by a reverse transcriptase). Class II transposable elements, or “DNA transposons”, are transposed via a complex of single- or double-stranded DNA flanked on either side by a transposase. Further features of this family of enzymes can be found, e.g. in Nature Education 2008, 1 (1), 204; and Genome Biology 2018, 19 (199), 1-12; each of which is incorporated herein by reference.

As used herein, the term “TnpA” generally refers to the transposase found in members of the IS200/IS605 bacterial insertion sequence (“IS”) family. Unlike other documented IS transposases, which carry out DNA transposition via double-stranded DNA intermediates, TnpA proceeds via a single-stranded DNA intermediate. TnpA also differs from other documented IS transposases in that it contains flanking subterminal palindromic sequences rather than terminal inverted repeats. Further, TnpA inserts 3′ to specific AT-rich tetra- or pentanucleotides without duplication of the target site. Finally, TnpA belongs to the His-hydrophobic-His (“HuH”) superfamily of enzymes rather than the “DDE” superfamily of other IS transposases. As used herein, “TnpB” generally refers to an enzyme of undocumented function (though speculated to play a regulatory role in transposition) found alongside TnpA in IS200/IS605 bacteria. IS200/IS605 transposases are “Y1 transposases”, meaning that they are single-domain proteins comprising a single catalytic tyrosine residue. As used herein, the term “TnpA-like” generally refers to a protein which exhibits one or more functional, structural, biochemical, biophysical, or other properties or characteristics in common with a TnpA protein. As used herein, the term “TnpB-like” generally refers to a protein which exhibits one or more function, structural, biochemical, biophysical, or other properties or characteristics in common with a TnpB protein.

The term “sequence identity” or “percent identity” in the context of two or more nucleic acids or polypeptide sequences, generally refers to two (e.g., in a pairwise alignment) or more (e.g., in a multiple sequence alignment) sequences that are the same or have a specified percentage of amino acid residues or nucleotides that are the same, when compared and aligned for maximum correspondence over a local or global comparison window, as measured using a sequence comparison algorithm. Suitable sequence comparison algorithms for polypeptide sequences include, e.g., BLASTP using parameters of a wordlength (W) of 3, an expectation (E) of 10, and the BLOSUM62 scoring matrix setting gap costs at existence of 11, extension of 1, and using a conditional compositional score matrix adjustment for polypeptide sequences longer than 30 residues; BLASTP using parameters of a wordlength (W) of 2, an expectation (E) of 1000000, and the PAM30 scoring matrix setting gap costs at 9 to open gaps and 1 to extend gaps for sequences of less than 30 residues (these are the default parameters for BLASTP in the BLAST suite available at https://blast.ncbi.nlm.nih.gov); CLUSTALW with the Smith-Waterman homology search algorithm parameters with a match of 2, a mismatch of −1, and a gap of −1; MUSCLE with default parameters; MAFFT with parameters of a retree of 2 and max iterations of 1000; Novafold with default parameters; HMMER hmmalign with default parameters.

The term “optimally aligned” in the context of two or more nucleic acids or polypeptide sequences, generally refers to two (e.g., in a pairwise alignment) or more (e.g., in a multiple sequence alignment) sequences that have been aligned to maximal correspondence of amino acids residues or nucleotides, for example, as determined by the alignment producing a highest or “optimized” percent identity score.

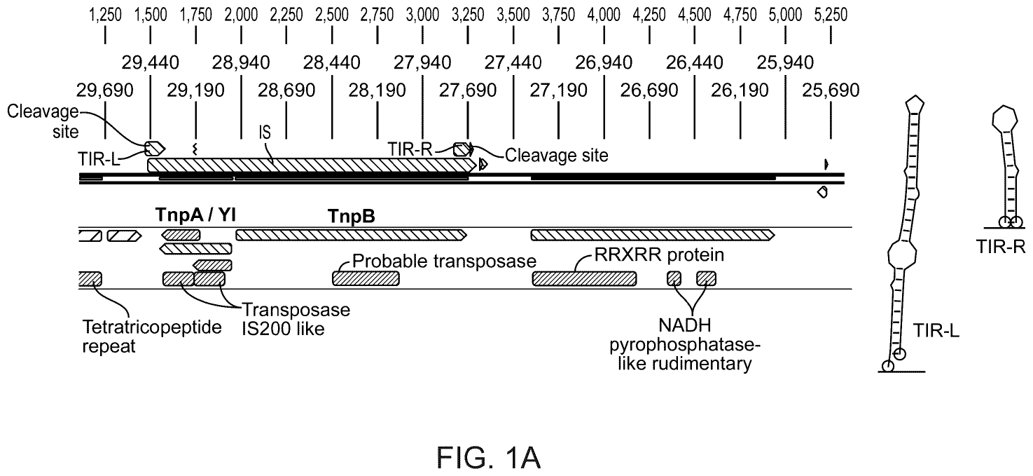

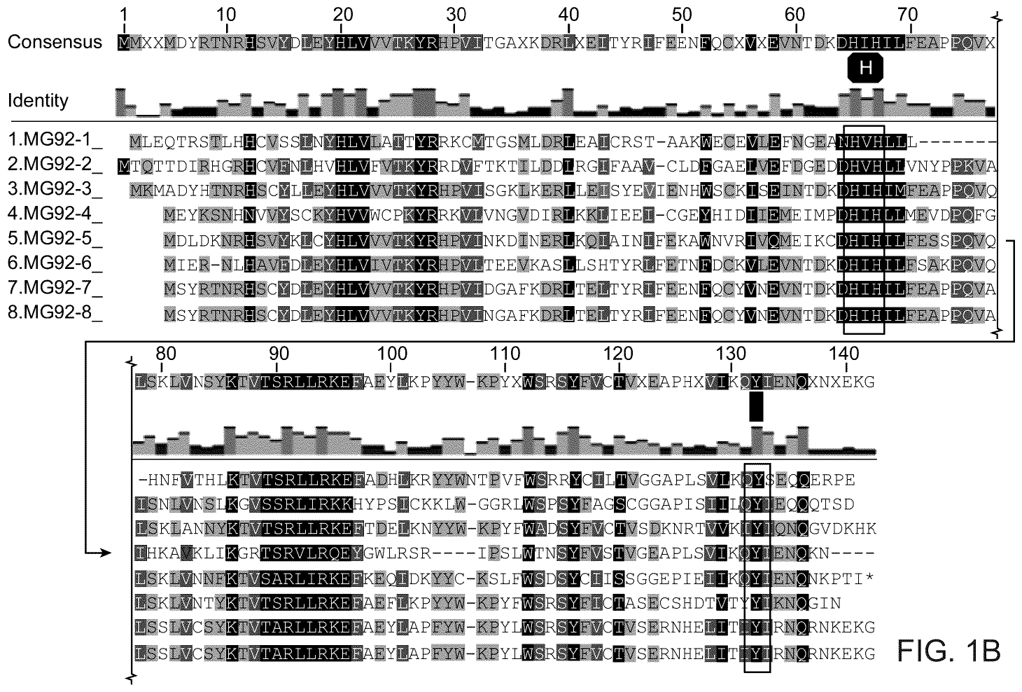

Included in the current disclosure are variants of any of the enzymes described herein with one or more conservative amino acid substitutions. Such conservative substitutions can be made in the amino acid sequence of a polypeptide without disrupting the three-dimensional structure or function of the polypeptide. Conservative substitutions can be accomplished by substituting amino acids with similar hydrophobicity, polarity, and R chain length for one another. Additionally, or alternatively, by comparing aligned sequences of homologous proteins from different species, conservative substitutions can be identified by locating amino acid residues that have been mutated between species (e.g., non-conserved residues) without altering the basic functions of the encoded proteins. Such conservatively substituted variants may include variants with at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99% identity to any one of the transposase protein sequences described herein (e.g. MG92 family transposases described herein, or any other family transposase described herein). In some embodiments, such conservatively substituted variants are functional variants. Such functional variants can encompass sequences with substitutions such that the activity of one or more critical active site residues of the transposase are not disrupted. In some embodiments, a functional variant of any of the proteins described herein lacks substitution of at least one of the conserved or functional residues called out in FIG. 1B. In some embodiments, a functional variant of any of the proteins described herein lacks substitution of all of the conserved or functional residues called out in FIG. 1B.

Also included in the current disclosure are variants of any of the enzymes described herein with substitution of one or more catalytic residues to decrease or eliminate activity of the enzyme (e.g. decreased-activity variants). In some embodiments, a decreased activity variant as a protein described herein comprises a disrupting substitution of at least one, at least two, or all three catalytic residues called out in FIG. 1B.

Conservative substitution tables providing functionally similar amino acids are available from a variety of references (see, for e.g., Creighton, Proteins: Structures and Molecular Properties (W H Freeman & Co.; 2nd edition (December 1993)). The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M)

### Overview

The discovery of new transposable elements with unique functionality and structure may offer the potential to further disrupt deoxyribonucleic acid (DNA) editing technologies, improving speed, specificity, functionality, and ease of use. Relative to the predicted prevalence of transposable elements in microbes and the sheer diversity of microbial species, relatively few functionally characterized transposable elements exist in the literature. This is partly because a huge number of microbial species may not be readily cultivated in laboratory conditions. Metagenomic sequencing from natural environmental niches containing large numbers of microbial species may offer the potential to drastically increase the number of new transposable elements documented and speed the discovery of new oligonucleotide editing functionalities.

Transposable elements are deoxyribonucleic acid sequences that can change position within a genome, often resulting in the generation or amelioration of mutations. In eukaryotes, a great proportion of the genome, and a large share of the mass of cellular DNA, is attributable to transposable elements. Although transposable elements are “selfish genes” which propagate themselves at the expense of other genes, they have been found to serve various important functions and to be crucial to genome evolution. Based on their mechanism, transposable elements are classified as either Class I “retrotransposons” or Class II “DNA transposons”.

Class I transposable elements, also referred to as retrotransposons, function according to a two-part “copy and paste” mechanism involving an RNA intermediate. First, the retrotransposon is transcribed. The resulting RNA is subsequently converted back to DNA by reverse transcriptase (generally encoded by the retrotransposon itself), and the reverse transcribed retrotransposon is finally integrated into its new position in the genome by integrase. Retrotransposons are further classified into three orders. Retrotransposons with long terminal repeats (“LTRs”) encode reverse transcriptase and are flanked by long strands of repeating DNA. Retrotransposons with long interspersed nuclear elements (“LINEs”) encode reverse transcriptase, lack LTRs, and are transcribed by RNA polymerase II. Retrotransposons with short interspersed nuclear elements (“SINEs”) are transcribed by RNA polymerase III but lack reverse transcriptase, instead relying on the reverse transcription machinery of other transposable elements (e.g. LINEs).

Class II transposable elements, also referred to as DNA transposons, function according to mechanisms that do not involve an RNA intermediate. Many DNA transposons display a “cut and paste” mechanism in which transposase binds terminal inverted repeats (“TIRs”) flanking the transposon, cleaves the transposon from the donor region, and inserts it into the target region of the genome. Others, referred to as “helitrons”, display a “rolling circle” mechanism involving a single-stranded DNA intermediate and mediated by an undocumented protein believed to possess HUH endonuclease function and 5′ to 3′ helicase activity. First, a circular strand of DNA is nicked to create two single DNA strands. The protein remains attached to the 5′ phosphate of the nicked strand, leaving the 3′ hydroxyl end of the complementary strand exposed and thus allowing a polymerase to replicate the non-nicked strand. Once replication is complete, the new strand disassociates and is itself replicated along with the original template strand. Still other DNA transposons, “Polintons”, are theorized to undergo a “self-synthesis” mechanism. The transposition is initiated by an integrase's excision of a single-stranded extra-chromosomal Polinton element, which forms a racket-like structure. The Polinton undergoes replication with DNA polymerase B, and the double stranded Polinton is inserted into the genome by the integrase. Finally, some DNA transposons, such as those in the IS200/IS605 family, proceed via a “peel and paste” mechanism in which TnpA excises a piece of single-stranded DNA (as a circular “transposon joint”) from the lagging strand template of the donor gene and reinserts it into the replication fork of the target gene.

While transposable elements have found some use as biological tools, documented transposable elements do not encompass the full range of possible biodiversity and targetability, and may not represent all possible activities. Here, thousands of genomic fragments were mined from numerous metagenomes for transposable elements. The documented diversity of transposable elements may have been expanded and novel systems may have been developed into highly targetable, compact, and precise gene editing agents.

### MG Enzymes

In some aspects, the present disclosure provides for novel transposases. These candidates may represent one or more novel subtypes and some sub-families may have been identified. These transposases are less than about 500 amino acids in length. These transposases may simplify delivery and may extend therapeutic applications.

In some aspects, the present disclosure provides for a novel transposase. Such a transposase may be MG92 as described herein (see FIGS. 1A and 1B).

In one aspect, the present disclosure provides for an engineered transposase system discovered through metagenomic sequencing. In some embodiments, the metagenomic sequencing is conducted on samples. In some embodiments, the samples may be collected from a variety of environments. Such environments may be a human microbiome, an animal microbiome, environments with high temperatures, environments with low temperatures. Such environments may include sediment.

In one aspect, the present disclosure provides for an engineered transposase system comprising a transposase. In some embodiments, the transposase is derived from an uncultivated microorganism. The transposase may be configured to bind a left-hand region comprising a subterminal palindromic sequence. The transposase may bind a right-hand region comprising a subterminal palindromic sequence.

In one aspect, the present disclosure provides for an engineered transposase system comprising a transposase. In some embodiments, the transposase has at least about 70% sequence identity to any one of SEQ ID NOs: 1-349. In some embodiments, the transposase has at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 1-349.

In some embodiments, the transposase comprises a variant having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 1-349. In some embodiments, the transposase may be substantially identical to any one of SEQ ID NOs: 1-349.

In some embodiments, the transposase is not a TnpA or TnpB transposase. In some embodiments, the transposase has less than about 90%, less than about 85%, less than about 80%, less than about 75%, less than about 70%, less than about 65%, less than about 60%, less than about 55%, less than about 50%, less than about 45%, less than about 40%, less than about 35%, less than about 30%, less than about 25%, less than about 20%, less than about 15%, less than about 10%, or less than about 5% sequence identity to a TnpA transposase. In some embodiments, the transposase has less than about 90%, less than about 85%, less than about 80%, less than about 75%, less than about 70%, less than about 65%, less than about 60%, less than about 55%, less than about 50%, less than about 45%, less than about 40%, less than about 35%, less than about 30%, less than about 25%, less than about 20%, less than about 15%, less than about 10%, or less than about 5% sequence identity to a TnpB transposase.

In some embodiments, the transposase comprises a catalytic tyrosine residue.

In some embodiments, the transposase is configured to bind a left-hand region comprising a subterminal palindromic sequence. In some embodiments, the transposase is configured to bind a right-hand region comprising a subterminal palindromic sequence. In some embodiments, the transposase is configured to bind a left-hand region comprising a subterminal palindromic sequence and a right-hand region comprising a subterminal palindromic sequence.

In some embodiments, the transposase is configured to transpose the cargo nucleotide sequence as double-stranded deoxyribonucleic acid polynucleotide. In some embodiments, the transposase is configured to transpose the cargo nucleotide sequence as single-stranded deoxyribonucleic acid polynucleotide.

In some embodiments, the transposase comprises a sequence complementary to a eukaryotic, fungal, plant, mammalian, or human genomic polynucleotide sequence. In some embodiments, the transposase comprises a sequence complementary to a eukaryotic genomic polynucleotide sequence. In some embodiments, the transposase comprises a sequence complementary to a fungal genomic polynucleotide sequence. In some embodiments, the transposase comprises a sequence complementary to a plant genomic polynucleotide sequence. In some embodiments, the transposase comprises a sequence complementary to a mammalian genomic polynucleotide sequence. In some embodiments, the transposase comprises a sequence complementary to a human genomic polynucleotide sequence.

In some embodiments, the transposase may comprise a variant having one or more nuclear localization sequences (NLSs). The NLS may be proximal to the N- or C-terminus of the transposase. The NLS may be appended N-terminal or C-terminal to any one of SEQ ID NOs: 455-470, or to a variant having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 455-470. In some embodiments, the NLS may comprise a sequence substantially identical to any one of SEQ ID NOs: 455-470. In some embodiments, the NLS may comprise a sequence substantially identical to SEQ ID NO: 455. In some embodiments, the NLS may comprise a sequence substantially identical to SEQ ID NO: 456.

TABLE 1
Example NLS Sequences that may be used with
transposases according to the disclosure

| | | SEQ |
|---|---|---|
| Source | NLS amino acid sequence | ID NO: |
| SV40 | PKKKRKV | 455 |
| nucleoplasmin | KRPAATKKAGQAKKKK | 456 |
| bipartite NLS | | |
| c-myc NLS | PAAKRVKLD | 457 |
| c-myc NLS | RQRRNELKRSP | 458 |
| hRNPA1 M9 NLS | NQSSNFGPMKGGNFGGRSSGPYG | 459 |
| | GGGQYFAKPRNQGGY | |
| Importin-alpha IBB | RMRIZFKNKGKDTAELRRRRVEV | 460 |
| domain | SVELRKAKKDEQILKRRNV | |
| Myoma T protein | VSRKRPRP | 461 |
| Myoma T protein | PPKKARED | 462 |
| p53 | PQPKKKPL | 463 |
| mouse c-abl IV | SALIKKKKKMAP | 464 |
| influenza virus NS1 | DRLRR | 465 |
| influenza virus NS1 | PKQKKRK | 466 |
| Hepatitis virus | RKLKKKIKKL | 467 |
| delta antigen | | |
| mouse Mx1 protein | REKKKELKRR | 468 |
| human poly(ADP- | KRKGDEVDGVDEVAKKKSKK | 469 |
| ribose) polymerase | | |
| steroid hormone | RKCLQAGMNLEARKTKK | 470 |
| receptors (human) | | |
| glucocorticoid | | |

In some embodiments, the transposase comprises a sequence at least 70% identical to a variant of any one of SEQ ID NOs: 1, 3, 5, 7, 9, 11, 13, 15, or 16, or a variant thereof. In some embodiments, the transposase comprises a sequence at least 75% identical to a variant of any one of SEQ ID NOs: 1, 3, 5, 7, 9, 11, 13, 15, or 16, or a variant thereof. In some embodiments, the transposase comprises a sequence at least 80% identical to a variant of any one of SEQ ID NOs: 1, 3, 5, 7, 9, 11, 13, 15, or 16, or a variant thereof. In some embodiments, the transposase comprises a sequence at least 85% identical to a variant of any one of SEQ ID NOs: 1, 3, 5, 7, 9, 11, 13, 15, or 16, or a variant thereof. In some embodiments, the transposase comprises a sequence at least 90% identical to a variant of any one of SEQ ID NOs: 1, 3, 5, 7, 9, 11, 13, 15, or 16, or a variant thereof. In some embodiments, the transposase comprises a sequence at least 95% identical to a variant of any one of SEQ ID NOs: 1, 3, 5, 7, 9, 11, 13, 15, or 16, or a variant thereof.

In some embodiments, the transposase comprises a sequence at least 70% identical to a variant of any one of SEQ ID NOs: 2, 4, 6, 8, 10, 12, 14, or 17, or a variant thereof. In some embodiments, the transposase comprises a sequence at least 75% identical to a variant of any one of SEQ ID NOs: 2, 4, 6, 8, 10, 12, 14, or 17, or a variant thereof. In some embodiments, the transposase comprises a sequence at least 80% identical to a variant of any one of SEQ ID NOs: 2, 4, 6, 8, 10, 12, 14, or 17, or a variant thereof. In some embodiments, the transposase comprises a sequence at least 85% identical to a variant of any one of SEQ ID NOs: 2, 4, 6, 8, 10, 12, 14, or 17, or a variant thereof. In some embodiments, the transposase comprises a sequence at least 90% identical to a variant of any one of SEQ ID NOs: 2, 4, 6, 8, 10, 12, 14, or 17, or a variant thereof. In some embodiments, the transposase comprises a sequence at least 95% identical to a variant of any one of SEQ ID NOs: 2, 4, 6, 8, 10, 12, 14, or 17, or a variant thereof.

In some embodiments, sequence may be determined by a BLASTP, CLUSTALW, MUSCLE, or MAFFT algorithm, or a CLUSTALW algorithm with the Smith-Waterman homology search algorithm parameters. The sequence identity may be determined by the BLASTP homology search algorithm using parameters of a wordlength (W) of 3, an expectation (E) of 10, and a BLOSUM62 scoring matrix setting gap costs at existence of 11, extension of 1, and using a conditional compositional score matrix adjustment.

In one aspect, the present disclosure provides a deoxyribonucleic acid polynucleotide encoding the engineered transposase system described herein.

In one aspect, the present disclosure provides a nucleic acid comprising an engineered nucleic acid sequence. In some embodiments, the engineered nucleic acid sequence is optimized for expression in an organism. In some embodiments, the transposase is derived from an uncultivated microorganism. In some embodiments, the organism is not the uncultivated organism.

In some embodiments, the transposase has at least about 70% sequence identity to any one of SEQ ID NOs: 1-349. In some embodiments, the transposase has at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 1-349.

In some embodiments, the transposase comprises a variant having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 1-349. In some embodiments, the transposase may be substantially identical to any one of SEQ ID NOs: 1-349.

In some embodiments, the transposase is not a TnpA or TnpB transposase. In some embodiments, the transposase has less than about 90%, less than about 85%, less than about 80%, less than about 75%, less than about 70%, less than about 65%, less than about 60%, less than about 55%, less than about 50%, less than about 45%, less than about 40%, less than about 35%, less than about 30%, less than about 25%, less than about 20%, less than about 15%, less than about 10%, or less than about 5% sequence identity to a TnpA transposase. In some embodiments, the transposase has less than about 90%, less than about 85%, less than about 80%, less than about 75%, less than about 70%, less than about 65%, less than about 60%, less than about 55%, less than about 50%, less than about 45%, less than about 40%, less than about 35%, less than about 30%, less than about 25%, less than about 20%, less than about 15%, less than about 10%, or less than about 5% sequence identity to a TnpB transposase.

In some embodiments, the transposase comprises a catalytic tyrosine residue.

In some embodiments, the transposase is configured to bind a left-hand region comprising a subterminal palindromic sequence. In some embodiments, the transposase is configured to bind a right-hand region comprising a subterminal palindromic sequence. In some embodiments, the transposase is configured to bind a left-hand region comprising a subterminal palindromic sequence and a right-hand region comprising a subterminal palindromic sequence.

In some embodiments, the transposase is configured to transpose the cargo nucleotide sequence as double-stranded deoxyribonucleic acid polynucleotide. In some embodiments, the transposase is configured to transpose the cargo nucleotide sequence as single-stranded deoxyribonucleic acid polynucleotide.

In some embodiments, the transposase comprises a sequence complementary to a eukaryotic, fungal, plant, mammalian, or human genomic polynucleotide sequence. In some embodiments, the transposase comprises a sequence complementary to a eukaryotic genomic polynucleotide sequence. In some embodiments, the transposase comprises a sequence complementary to a fungal genomic polynucleotide sequence. In some embodiments, the transposase comprises a sequence complementary to a plant genomic polynucleotide sequence. In some embodiments, the transposase comprises a sequence complementary to a mammalian genomic polynucleotide sequence. In some embodiments, the transposase comprises a sequence complementary to a human genomic polynucleotide sequence.

In some embodiments, the transposase may comprise a variant having one or more nuclear localization sequences (NLSs). The NLS may be proximal to the N- or C-terminus of the transposase. The NLS may be appended N-terminal or C-terminal to any one of SEQ ID NOs: 455-470, or to a variant having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 455-470. In some embodiments, the NLS may comprise a sequence substantially identical to any one of SEQ ID NOs: 455-470. In some embodiments, the NLS may comprise a sequence substantially identical to SEQ ID NO: 455. In some embodiments, the NLS may comprise a sequence substantially identical to SEQ ID NO: 456.

In some embodiments, the organism is prokaryotic. In some embodiments, the organism is bacterial. In some embodiments, the organism is eukaryotic. In some embodiments, the organism is fungal. In some embodiments, the organism is a plant. In some embodiments, the organism is mammalian. In some embodiments, the organism is a rodent. In some embodiments, the organism is human.

In one aspect, the present disclosure provides an engineered vector. In some embodiments, the engineered vector comprises a nucleic acid sequence encoding a transposase. In some embodiments, the transposase is derived from an uncultivated microorganism.

In some embodiments, the engineered vector comprises a nucleic acid described herein. In some embodiments, the nucleic acid described herein is a deoxyribonucleic acid polynucleotide described herein. In some embodiments, the vector is a plasmid, a minicircle, a CELiD, an adeno-associated virus (AAV) derived virion, or a lentivirus.

In one aspect, the present disclosure provides a cell comprising a vector described herein.

In one aspect, the present disclosure provides a method of manufacturing a transposase. In some embodiments, the method comprises cultivating the cell.

In one aspect, the present disclosure provides a method for binding, nicking, cleaving, marking, modifying, or transposing a double-stranded deoxyribonucleic acid polynucleotide. The method may comprise contacting the double-stranded deoxyribonucleic acid polynucleotide with a transposase. In some embodiments, the transposase is configured to bind a left-hand region comprising a subterminal palindromic sequence. In some embodiments, the transposase is configured to bind a right-hand region comprising a subterminal palindromic sequence. In some embodiments, the transposase is configured to bind a left-hand region comprising a subterminal palindromic sequence and a right-hand region comprising a subterminal palindromic sequence.

In some embodiments, the transposase is not a TnpA transposase or a TnpB transposase. In some embodiments, the transposase has less than about 90%, less than about 85%, less than about 80%, less than about 75%, less than about 70%, less than about 65%, less than about 60%, less than about 55%, less than about 50%, less than about 45%, less than about 40%, less than about 35%, less than about 30%, less than about 25%, less than about 20%, less than about 15%, less than about 10%, or less than about 5% sequence identity to a TnpA transposase. In some embodiments, the transposase has less than about 90%, less than about 85%, less than about 80%, less than about 75%, less than about 70%, less than about 65%, less than about 60%, less than about 55%, less than about 50%, less than about 45%, less than about 40%, less than about 35%, less than about 30%, less than about 25%, less than about 20%, less than about 15%, less than about 10%, or less than about 5% sequence identity to a TnpB transposase.

In some embodiments, the transposase comprises a catalytic tyrosine residue. In some embodiments, the transposase is configured to transpose the cargo nucleotide sequence as double-stranded deoxyribonucleic acid polynucleotide. In some embodiments, the transposase is configured to transpose the cargo nucleotide sequence as single-stranded deoxyribonucleic acid polynucleotide.

In some embodiments, the transposase is derived from an uncultivated microorganism. In some embodiments, the double-stranded deoxyribonucleic acid polynucleotide is a eukaryotic, plant, fungal, mammalian, rodent, or human double-stranded deoxyribonucleic acid polynucleotide.

In one aspect, the present disclosure provides a method of modifying a target nucleic acid locus. The method may comprise delivering to the target nucleic acid locus the engineered transposase system described herein. In some embodiments, the complex is configured such that upon binding of the complex to the target nucleic acid locus, the complex modifies the target nucleic acid locus.

In some embodiments, modifying the target nucleic acid locus comprises binding, nicking, cleaving, marking, modifying, or transposing the target nucleic acid locus. In some embodiments, the target nucleic acid locus comprises deoxyribonucleic acid (DNA) or ribonucleic acid (RNA). In some embodiments, the target nucleic acid comprises genomic DNA, viral DNA, viral RNA, or bacterial DNA. In some embodiments, the target nucleic acid locus is in vitro. In some embodiments, the target nucleic acid locus is within a cell. In some embodiments, the cell is a prokaryotic cell, a bacterial cell, a eukaryotic cell, a fungal cell, a plant cell, an animal cell, a mammalian cell, a rodent cell, a primate cell, or a human cell. In some embodiments, the cell is a primary cell. In some embodiments, the primary cell is a T cell. In some embodiments, the primary cell is a hematopoietic stem cell (HSC).

In some embodiments, delivery of the engineered transposase system to the target nucleic acid locus comprises delivering the nucleic acid described herein or the vector described herein. In some embodiments, delivery of engineered transposase system to the target nucleic acid locus comprises delivering a nucleic acid comprising an open reading frame encoding the transposase. In some embodiments, the nucleic acid comprises a promoter. In some embodiments, the open reading frame encoding the transposase is operably linked to the promoter.

In some embodiments, delivery of the engineered transposase system to the target nucleic acid locus comprises delivering a capped mRNA containing the open reading frame encoding the transposase. In some embodiments, delivery of the engineered transposase system to the target nucleic acid locus comprises delivering a translated polypeptide. In some embodiments, delivery of the engineered transposase system to the target nucleic acid locus comprises delivering a deoxyribonucleic acid (DNA) encoding the engineered guide RNA operably linked to a ribonucleic acid (RNA) pol III promoter.

In some embodiments, the transposase induces a single-stranded break or a double-stranded break at or proximal to the target locus. In some embodiments, the transposase induces a staggered single stranded break within or 5′ to the target locus.

In one aspect, the present disclosure provides a host cell comprising an open reading frame encoding a heterologous transposase. In some embodiments, the transposase has at least about 70% sequence identity to any one of SEQ ID NOs: 1-349. In some embodiments, the transposase has at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 1-349.

In some embodiments, the transposase comprises a variant having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 1-349. In some embodiments, the transposase may be substantially identical to any one of SEQ ID NOs: 1-349.

In some embodiments, the transposase is not a TnpA or TnpB transposase. In some embodiments, the transposase has less than about 90%, less than about 85%, less than about 80%, less than about 75%, less than about 70%, less than about 65%, less than about 60%, less than about 55%, less than about 50%, less than about 45%, less than about 40%, less than about 35%, less than about 30%, less than about 25%, less than about 20%, less than about 15%, less than about 10%, or less than about 5% sequence identity to a TnpA transposase. In some embodiments, the transposase has less than about 90%, less than about 85%, less than about 80%, less than about 75%, less than about 70%, less than about 65%, less than about 60%, less than about 55%, less than about 50%, less than about 45%, less than about 40%, less than about 35%, less than about 30%, less than about 25%, less than about 20%, less than about 15%, less than about 10%, or less than about 5% sequence identity to a TnpB transposase.

In some embodiments, the transposase comprises a catalytic tyrosine residue.

In some embodiments, the transposase is configured to bind a left-hand region comprising a subterminal palindromic sequence. In some embodiments, the transposase is configured to bind a right-hand region comprising a subterminal palindromic sequence. In some embodiments, the transposase is configured to bind a left-hand region comprising a subterminal palindromic sequence and a right-hand region comprising a subterminal palindromic sequence.

In some embodiments, the transposase is configured to transpose the cargo nucleotide sequence as double-stranded deoxyribonucleic acid polynucleotide. In some embodiments, the transposase is configured to transpose the cargo nucleotide sequence as single-stranded deoxyribonucleic acid polynucleotide.

In some embodiments, the transposase comprises a sequence at least 70% identical to a variant of any one of SEQ ID NOs: 1, 3, 5, 7, 9, 11, 13, 15, or 16, or a variant thereof. In some embodiments, the transposase comprises a sequence at least 75% identical to a variant of any one of SEQ ID NOs: 1, 3, 5, 7, 9, 11, 13, 15, or 16, or a variant thereof. In some embodiments, the transposase comprises a sequence at least 80% identical to a variant of any one of SEQ ID NOs: 1, 3, 5, 7, 9, 11, 13, 15, or 16, or a variant thereof. In some embodiments, the transposase comprises a sequence at least 85% identical to a variant of any one of SEQ ID NOs: 1, 3, 5, 7, 9, 11, 13, 15, or 16, or a variant thereof. In some embodiments, the transposase comprises a sequence at least 90% identical to a variant of any one of SEQ ID NOs: 1, 3, 5, 7, 9, 11, 13, 15, or 16, or a variant thereof. In some embodiments, the transposase comprises a sequence at least 95% identical to a variant of any one of SEQ ID NOs: 1, 3, 5, 7, 9, 11, 13, 15, or 16, or a variant thereof.

In some embodiments, the transposase comprises a sequence at least 70% identical to a variant of any one of SEQ ID NOs: 2, 4, 6, 8, 10, 12, 14, or 17, or a variant thereof. In some embodiments, the transposase comprises a sequence at least 75% identical to a variant of any one of SEQ ID NOs: 2, 4, 6, 8, 10, 12, 14, or 17, or a variant thereof. In some embodiments, the transposase comprises a sequence at least 80% identical to a variant of any one of SEQ ID NOs: 2, 4, 6, 8, 10, 12, 14, or 17, or a variant thereof. In some embodiments, the transposase comprises a sequence at least 85% identical to a variant of any one of SEQ ID NOs: 2, 4, 6, 8, 10, 12, 14, or 17, or a variant thereof. In some embodiments, the transposase comprises a sequence at least 90% identical to a variant of any one of SEQ ID NOs: 2, 4, 6, 8, 10, 12, 14, or 17, or a variant thereof. In some embodiments, the transposase comprises a sequence at least 95% identical to a variant of any one of SEQ ID NOs: 2, 4, 6, 8, 10, 12, 14, or 17, or a variant thereof.

In some embodiments, the host cell is an E. coli cell. In some embodiments, the E. coli cell is a λDE3 lysogen or the E. coli cell is a BL21(DE3) strain. In some embodiments, the E. coli cell has an ompT lon genotype.

In some embodiments, the open reading frame is operably linked to a T7 promoter sequence, a T7-lac promoter sequence, a lac promoter sequence, a tac promoter sequence, a trc promoter sequence, a ParaBAD promoter sequence, a PrhaBAD promoter sequence, a T5 promoter sequence, a cspA promoter sequence, an araPBAD promoter, a strong leftward promoter from phage lambda (pL promoter), or any combination thereof.

In some embodiments, the open reading frame comprises a sequence encoding an affinity tag linked in-frame to a sequence encoding the transposase. In some embodiments, the affinity tag is an immobilized metal affinity chromatography (IMAC) tag. In some embodiments, the IMAC tag is a polyhistidine tag. In some embodiments, the affinity tag is a myc tag, a human influenza hemagglutinin (HA) tag, a maltose binding protein (MBP) tag, a glutathione S-transferase (GST) tag, a streptavidin tag, a FLAG tag, or any combination thereof. In some embodiments, the affinity tag is linked in-frame to the sequence encoding the transposase via a linker sequence encoding a protease cleavage site. In some embodiments, the protease cleavage site is a tobacco etch virus (TEV) protease cleavage site, a PreScission® protease cleavage site, a Thrombin cleavage site, a Factor Xa cleavage site, an enterokinase cleavage site, or any combination thereof.

In some embodiments, the open reading frame is codon-optimized for expression in the host cell. In some embodiments, the open reading frame is provided on a vector. In some embodiments, the open reading frame is integrated into a genome of the host cell.

In one aspect, the present disclosure provides a culture comprising a host cell described herein in compatible liquid medium.

In one aspect, the present disclosure provides a method of producing a transposase, comprising cultivating a host cell described herein in compatible growth medium. In some embodiments, the method further comprises inducing expression of the transposase by addition of an additional chemical agent or an increased amount of a nutrient. In some embodiments, the additional chemical agent or increased amount of a nutrient comprises Isopropyl j-D-1-thiogalactopyranoside (IPTG) or additional amounts of lactose. In some embodiments, the method further comprises isolating the host cell after the cultivation and lysing the host cell to produce a protein extract. In some embodiments, the method further comprises subjecting the protein extract to IMAC, or ion-affinity chromatography. In some embodiments, the open reading frame comprises a sequence encoding an IMAC affinity tag linked in-frame to a sequence encoding the transposase. In some embodiments, the IMAC affinity tag is linked in-frame to the sequence encoding the transposase via a linker sequence encoding protease cleavage site. In some embodiments, the protease cleavage site comprises a tobacco etch virus (TEV) protease cleavage site, a PreScission® protease cleavage site, a Thrombin cleavage site, a Factor Xa cleavage site, an enterokinase cleavage site, or any combination thereof. In some embodiments, the method further comprises cleaving the IMAC affinity tag by contacting a protease corresponding to the protease cleavage site to the transposase. In some embodiments, the method further comprises performing subtractive IMAC affinity chromatography to remove the affinity tag from a composition comprising the transposase.

In one aspect, the present disclosure provides a method of disrupting a locus in a cell. In some embodiments, the method comprises contacting to the cell a composition comprising a transposase. In some embodiments, the transposase has at least equivalent transposition activity to TnpA transposase in a cell. In some embodiments, the transposase has at least about 70% sequence identity to any one of SEQ ID NOs: 1-349. In some embodiments, the transposase has at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 1-349.

In some embodiments, the transposase comprises a variant having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 1-349. In some embodiments, the transposase may be substantially identical to any one of SEQ ID NOs: 1-349.

In some embodiments, the transposase is not a TnpA or TnpB transposase. In some embodiments, the transposase has less than about 90%, less than about 85%, less than about 80%, less than about 75%, less than about 70%, less than about 65%, less than about 60%, less than about 55%, less than about 50%, less than about 45%, less than about 40%, less than about 35%, less than about 30%, less than about 25%, less than about 20%, less than about 15%, less than about 10%, or less than about 5% sequence identity to a TnpA transposase. In some embodiments, the transposase has less than about 90%, less than about 85%, less than about 80%, less than about 75%, less than about 70%, less than about 65%, less than about 60%, less than about 55%, less than about 50%, less than about 45%, less than about 40%, less than about 35%, less than about 30%, less than about 25%, less than about 20%, less than about 15%, less than about 10%, or less than about 5% sequence identity to a TnpB transposase.

In some embodiments, the transposase comprises a catalytic tyrosine residue.

In some embodiments, the transposase is configured to bind a left-hand region comprising a subterminal palindromic sequence. In some embodiments, the transposase is configured to bind a right-hand region comprising a subterminal palindromic sequence. In some embodiments, the transposase is configured to bind a left-hand region comprising a subterminal palindromic sequence and a right-hand region comprising a subterminal palindromic sequence.

In some embodiments, the transposase is configured to transpose the cargo nucleotide sequence as double-stranded deoxyribonucleic acid polynucleotide. In some embodiments, the transposase is configured to transpose the cargo nucleotide sequence as single-stranded deoxyribonucleic acid polynucleotide.

In some embodiments, the transposase comprises a sequence complementary to a eukaryotic, fungal, plant, mammalian, or human genomic polynucleotide sequence. In some embodiments, the transposase comprises a sequence complementary to a eukaryotic genomic polynucleotide sequence. In some embodiments, the transposase comprises a sequence complementary to a fungal genomic polynucleotide sequence. In some embodiments, the transposase comprises a sequence complementary to a plant genomic polynucleotide sequence. In some embodiments, the transposase comprises a sequence complementary to a mammalian genomic polynucleotide sequence. In some embodiments, the transposase comprises a sequence complementary to a human genomic polynucleotide sequence.

In some embodiments, the transposase may comprise a variant having one or more nuclear localization sequences (NLSs). The NLS may be proximal to the N- or C-terminus of the transposase. The NLS may be appended N-terminal or C-terminal to any one of SEQ ID NOs: 455-470, or to a variant having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 455-470. In some embodiments, the NLS may comprise a sequence substantially identical to any one of SEQ ID NOs: 455-470. In some embodiments, the NLS may comprise a sequence substantially identical to SEQ ID NO: 455. In some embodiments, the NLS may comprise a sequence substantially identical to SEQ ID NO: 456.

In some embodiments, the transposition activity is measured in vitro by introducing the transposase to cells comprising the target nucleic acid locus and detecting transposition of the target nucleic acid locus in the cells. In some embodiments, the composition comprises 20 picomoles (pmol) or less of the transposase. In some embodiments, the composition comprises 1 pmol or less of the transposase.

Systems of the present disclosure may be used for various applications, such as, for example, nucleic acid editing (e.g., gene editing), binding to a nucleic acid molecule (e.g., sequence-specific binding). Such systems may be used, for example, for addressing (e.g., removing or replacing) a genetically inherited mutation that may cause a disease in a subject, inactivating a gene in order to ascertain its function in a cell, as a diagnostic tool to detect disease-causing genetic elements (e.g. via cleavage of reverse-transcribed viral RNA or an amplified DNA sequence encoding a disease-causing mutation), as deactivated enzymes in combination with a probe to target and detect a specific nucleotide sequence (e.g. sequence encoding antibiotic resistance int bacteria), to render viruses inactive or incapable of infecting host cells by targeting viral genomes, to add genes or amend metabolic pathways to engineer organisms to produce valuable small molecules, macromolecules, or secondary metabolites, to establish a gene drive element for evolutionary selection, to detect cell perturbations by foreign small molecules and nucleotides as a biosensor.

### Examples

In accordance with IUPAC conventions, the following abbreviations are used throughout the examples:
A=adenine
C=cytosine
G=guanine
T=thymine
R=adenine or guanine
Y=cytosine or thymine
S=guanine or cytosine
W=adenine or thymine
K=guanine or thymine
M=adenine or cytosine
B=C, G, or T
D=A, G, or T
H=A, C, or T
V=A, C, or G

### Example 1—A method of metagenomic analysis for new proteins

Metagenomic samples were collected from sediment, soil, and animals. Deoxyribonucleic acid (DNA) was extracted with a Zymobiomics DNA mini-prep kit and sequenced on an Illumina HiSeq® 2500. Samples were collected with consent of property owners. Additional raw sequence data from public sources included animal microbiomes, sediment, soil, hot springs, hydrothermal vents, marine, peat bogs, permafrost, and sewage sequences. Metagenomic sequence data was searched using Hidden Markov Models generated based on documented transposase protein sequences to identify new transposases. Novel transposase proteins identified by the search were aligned to documented proteins to identify potential active sites. This metagenomic workflow resulted in the delineation of the MG92 family described herein.

### Example 2—Discovery of MG92 Family of Transposases

Analysis of the data from the metagenomic analysis of Example 1 revealed a new cluster of previously undescribed putative transposase systems comprising 1 family (MG92). The corresponding protein sequences for these new enzymes and their example subdomains are presented as SEQ ID NOs: 1-349.

### Example 3-Integrase In Vitro Activity (Prophetic)

Integrase activity can be conducted via expression in an E. coli lysate based expression system (for example, myTXTL, Arbor Biosciences). The required components for in vitro testing are three plasmids: an expression plasmid with the transposon gene(s) under a T7 promoter, a target plasmid, and a donor plasmid which contains the required left end (LE) and right end (RE) DNA sequences for transposition around a cargo gene (e.g. Tet resistance gene). The lysate-based expression products, target DNA, and donor DNA are incubated to allow for transposition to occur. Transposition is detected via PCR. In addition, the transposition product will be tagmented with T5 and sequenced via NGS to determine the insertion sites on a population of transposition events. Alternatively, the in vitro transposition products can be transformed into E. coli under antibiotic (e.g. Tet) selection, where growth requires the transposition cargo to be stably inserted into a plasmid. Either single colonies or a population of E. coli can be sequenced to determine the insertion sites.

Integration efficiency can be measured via ddPCR or qPCR of the experimental output of target DNA with integrated cargo, normalized to the amount of unmodified target DNA also measured via ddPCR.

This assay may also be conducted with purified protein components rather than from lysate-based expression. In this case, the proteins are expressed in E. coli protease-deficient B strain under T7 inducible promoter, the cells are lysed using sonication, and the His-tagged protein of interest is purified using HisTrap FF (GE Lifescience) Ni-NTA affinity chromatography on the AKTA Avant FPLC (GE Lifescience). Purity is determined using densitometry in ImageLab software (Bio-Rad) of the protein bands resolved on SDS-PAGE and InstantBlue Ultrafast (Sigma-Aldrich) coomassie stained acrylamide gels (Bio-Rad). The protein is desalted in storage buffer composed of 50 mM Tris-HCl, 300 mM NaCl, 1 mM TCEP, 5% glycerol; pH 7.5 (or other buffers as determined for maximum stability) and stored at −80° C. After purification the transposon gene(s) are added to the target DNA and donor DNA as described above in a reaction buffer, for example 26 mM HEPES pH 7.5, 4.2 mM TRIS pH 8, 50 μg/mL BSA, 2 mM ATP, 2.1 mM DTT, 0.05 mM EDTA, 0.2 mM MgCl2, 28 mM NaCl, 21 mM KCl, 1.35% glycerol, (final pH 7.5) supplemented with 15 mM MgOAc₂.

### Example 4—Transposon End Verification Via Gel Shift (Prophetic)

The transposon ends are tested for transposase binding via an electrophoretic mobility shift assay (EMSA). In this case, the potential LE or RE is synthesized as a DNA fragment (100-500 bp) and end-labeled with FAM via PCR with FAM-labeled primers. The transposase protein is synthesized in an in vitro transcription/translation system (e.g. PURExpress). After synthesis, 1 μL of protein is added to 50 nM of the labeled RE or LE in a 10 uL reaction in binding buffer (e.g. 20 mM HEPES pH 7.5, 2.5 mM Tris pH 7.5, 10 mM NaCl, 0.0625 mM EDTA, 5 mM TCEP, 0.005% BSA, 1 μg/mL poly(dI-dC), and 5% glycerol). The binding is incubated at 30° for 40 minutes, then 2 μL of 6× loading buffer (60 mM KCl, 10 mM Tris pH 7.6, 50% glycerol) is added. The binding reaction is separated on a 5% TBE gel and visualized. Shifts of the LE or RE in the presence of transposase protein can be attributed to successful binding and are indicative of transposase activity. This assay can also be performed with transposase truncations or mutations, as well as using E. coli extract or purified protein.

### Example 5-Cleavage of Donor DNA Verification (Prophetic)

To confirm that the transposase is involved in cleavage of donor DNA, short (˜140 bp) fragments containing RE-LE junctions separated by up to 10 bp are labelled at both ends with FAM via PCR with FAM-labeled primers. Labeled DNA fragments are incubated with in vitro transcription/translation transposase products and the DNA is analyzed on a denaturing gel. Cleavage at each end of the junction can result in two labelled single-strand fragments which migrate at different rates on the gel.

### Example 6—Integrase Activity in E. coli (Prophetic)

Engineered E. coli strains are transformed with a plasmid expressing the transposon genes and a plasmid containing a temperature-sensitive origin of replication with a selectable marker flanked by left end (LE) and right end (RE) transposon motifs for integration. To confirm donor ssDNA preference by the transposase components, ssDNA plasmid supercoiling can be used as donor. Transformants induced for expression of these genes are then screened for transfer of the marker to a genomic target by selection at restrictive temperature for plasmid replication and the marker integration in the genome is confirmed by PCR.

Integrations are screened using an unbiased approach. In brief, purified gDNA is tagmented with Tn5, and DNA of interest is then PCR amplified using primers specific to the Tn5 tagmentation and the selectable marker. The amplicons are then prepared for NGS sequencing. Analysis of the resulting sequences is trimmed of the transposon sequences and flanking sequences are mapped to the genome to determine insertion position, and insertion rates are determined.

Alternatively, a polA mutant E. coli strain, MM383, which produces a DNA polymerase I (Poll) that is defective at 42° C., is used to detect integration as described previously (Brandsma et al., 1981). Resistance to a selectable marker after growth at 42° C. indicates incorporation of donor DNA into the chromosome. The pUC19 plasmid without donor is used as a control following growth for 24 hours at 42° C. without antibiotic selection.

E. coli strains that successfully grow in selection media are presumed to have integrated the donor DNA encoding the cargo resistance gene. Colonies growing in antibiotic selection plates are genotyped for cargo presence and NGS of whole genome sequence is performed.

### Example 7—Integrase Activity in Mammalian Cells (Prophetic)

To show targeting and cleavage activity in mammalian cells, each of the transposon proteins is purified with 2 NLS peptides on either terminus of the protein sequence. A plasmid containing a selectable neomycin resistance marker (NeoR) or a fluorescent marker flanked by the left end (LE) and right end (RE) motifs is synthesized. Cells are then transfected with the plasmid, recovered for 4-6 hours, and subsequently electroporated with transposon proteins. Antibiotic resistance integration into the genome is quantified by G418-resistant colony counts, and positive transposition by the fluorescent marker is assayed by fluorescence activated cell cytometry. 72 hours after cotransfection, genomic DNA is extracted and used for the preparation of an NGS-library. Integration frequency is assayed by Tn5 tagmentation.

### Example 8—in Silico Analysis

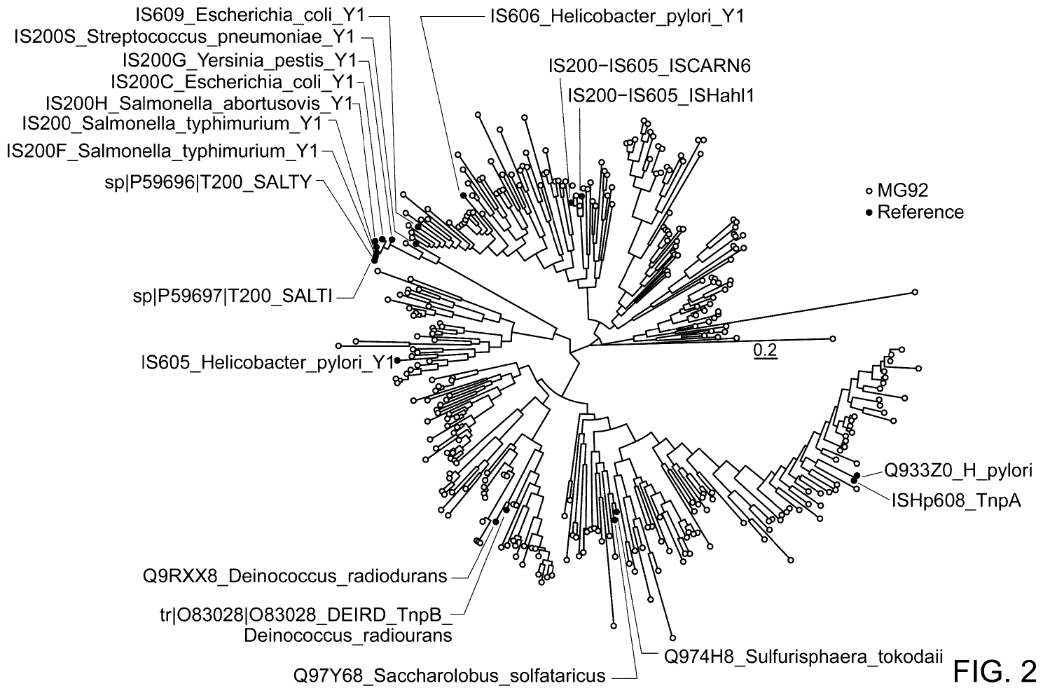

An extensive assembly-driven metagenomic database of microbial, viral and eukaryotic genomes was mined to retrieve predicted proteins with ssDNA transposase function. Over 400 predicted proteins had a significant e-value (<1×10⁻⁵) hit to TnpA transposases of the insertion sequences IS200/IS605. After filtering for complete ORFs and confirming presence of catalytic residues (Y1 and HuH), the TnpA-like protein sequences were aligned with MAFFT with parameters G-INSI (Mol Biol Evol 30, 772-780 (2013)) and the alignment was used to infer a phylogenetic tree with FastTree2 (Plos One 5, e9490 (2010)). Phylogenetic analysis of TnpA transposases uncovered high diversity of novel TnpA-like protein sequences associated with IS200/IS605 insertion sequences (FIG. 2).

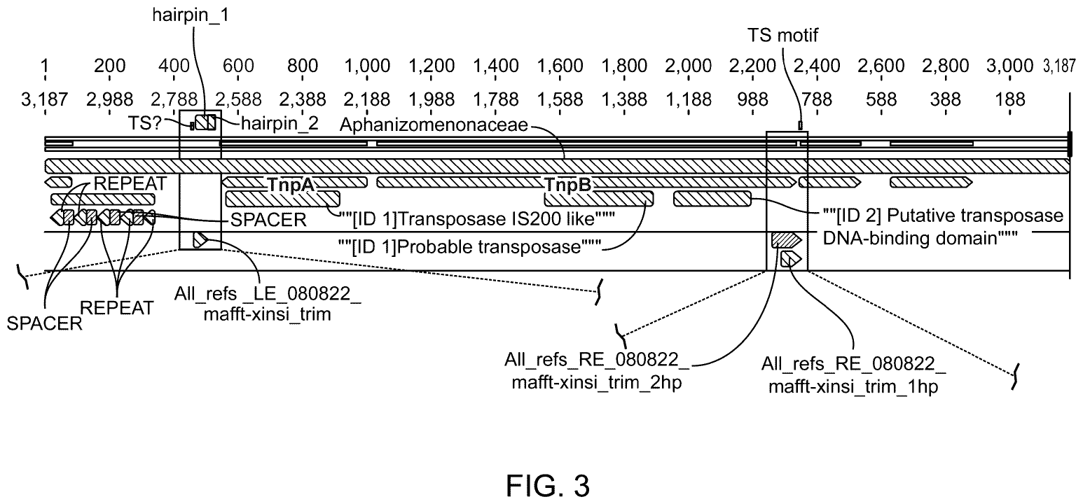

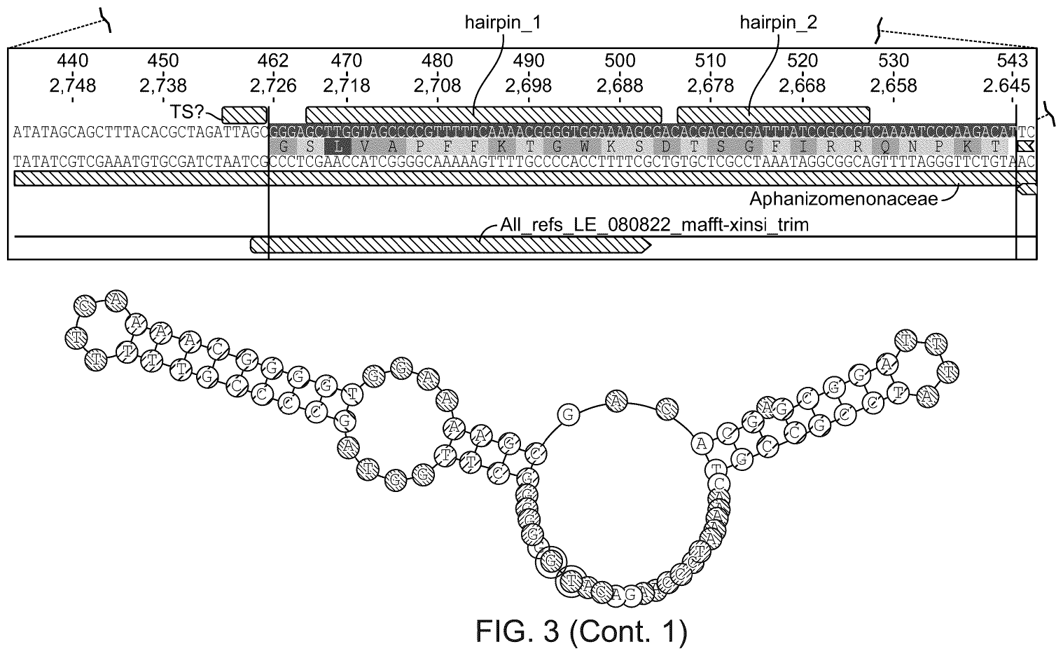

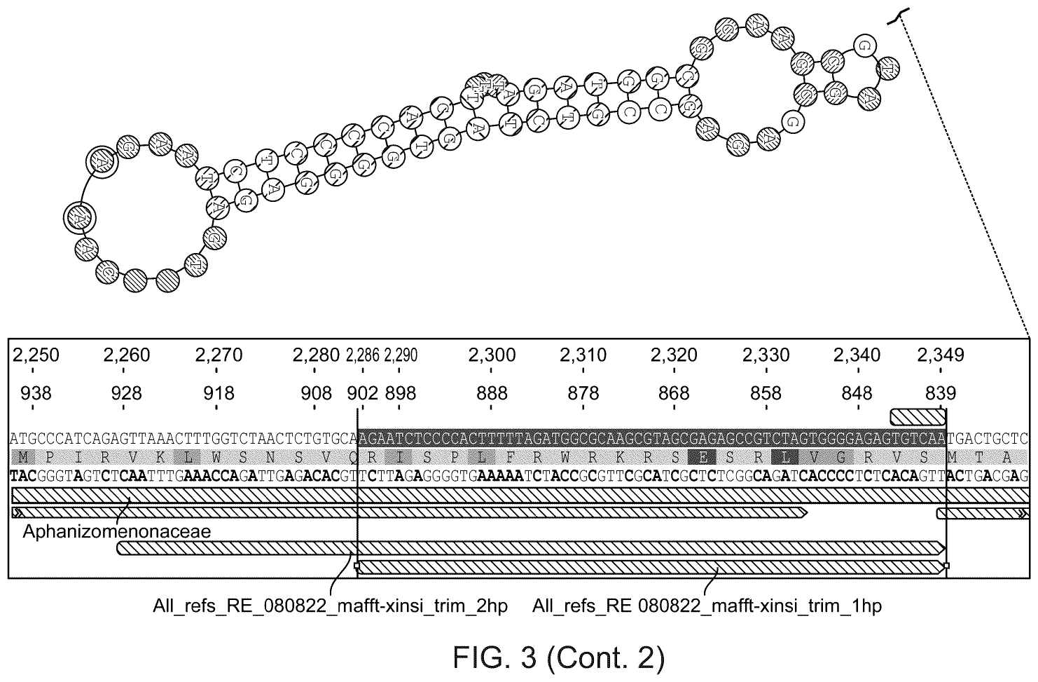

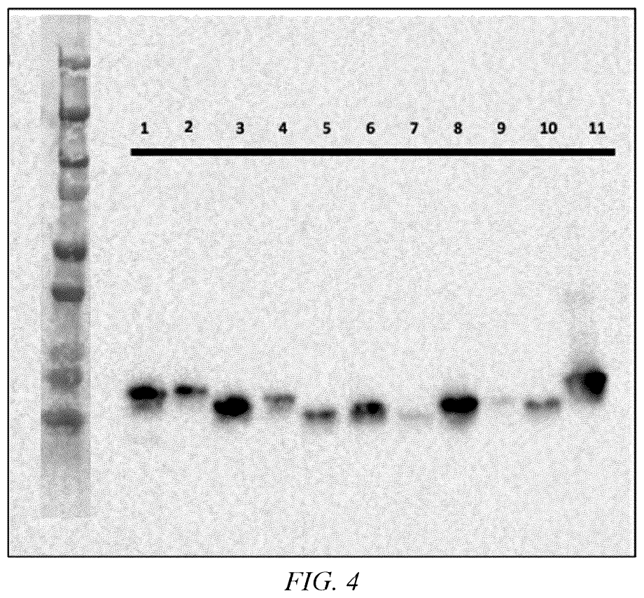

In order to predict the left and right ends (LE and RE) of the insertion sequence, covariance models were built from active LE and RE sequences available in the ISFinder database (https://www-is.biotoul.fr/). Specifically, a multiple sequence alignment (MSA) of LE and RE sequences was built with MAFFT with parameters X-INSI (Mol Biol Evol 30, 772-780 (2013)) and the secondary structure of the alignment was inferred from the MSA with RNAalifold 2.5.0 with parameters -p—aln-stk (Vienna Package). Covariance models were built with Infernal packages (http://eddylab.org/infernal/) and genomic fragments containing candidate TnpA transposases were searched using the covariance models with the Infernal command ‘cmsearch’. Covariance models predicted LE and RE for over 70 candidate IS200/IS605 insertion sequences (FIG. 3).

### Example 9—Generation of ssDNA Cargos

Each TnpA-like candidate had a unique cargo comprising the putative left end (LE) and right end (RE) sequences identified in the metagenomic contig. These putative LE and RE sequences were cloned to flank a kanamycin (Kan) resistance cargo gene via Gibson assembly. The ssDNA cargo was generated via PCR of the Kan cargo plasmid with common primers outside of the LE/RE regions with forward primer GTGCGGTAGTAAAGGTTAATACTGTT (SEQ ID NO: 471) and a 5′-phosphate-modified reverse primer CTATAGTGAGTCGTATTA (SEQ ID NO: 472) using standard cycling conditions with Phusion HF (NEB). After PCR amplification, the DNA bottom strand was degraded using Lambda exonuclease (NEB) and the remaining top strand was purified using a DCC-5 spin column with manufacturer's recommended changes for purifying ssDNA (Zymo Research). The single stranded DNA was checked on an agarose gel to verify complete conversion of dsDNA and quantified by the ssDNA Qubit kit (Thermofisher), yielding an average concentration of 20 nM.

### Example 10—Design of TnpA In Vitro Expression Constructs

For in vitro activity, each TnpA-like protein gene was synthesized in pET21(+) codon-optimized for E. coli translation under control of a T7 promoter and flanked by C-terminal HA and His tags, with the exception of 92-1 that lacks the HA tag. The TnpA-like protein plasmids were then amplified using primers that bind −150 bp upstream of the T7 promoter and downstream of the T7 terminator (primers TGGCGAGAAAGGAAGGGAAG (SEQ ID NO: 473) and CCGAAACAAGCGCTCATGAG (SEQ ID NO: 474)) and purified via SPRI bead clean-up (MagBio HighPrep) to give final template concentrations >80 ng/μL.

### Example 11—In Vitro Transposition Activity

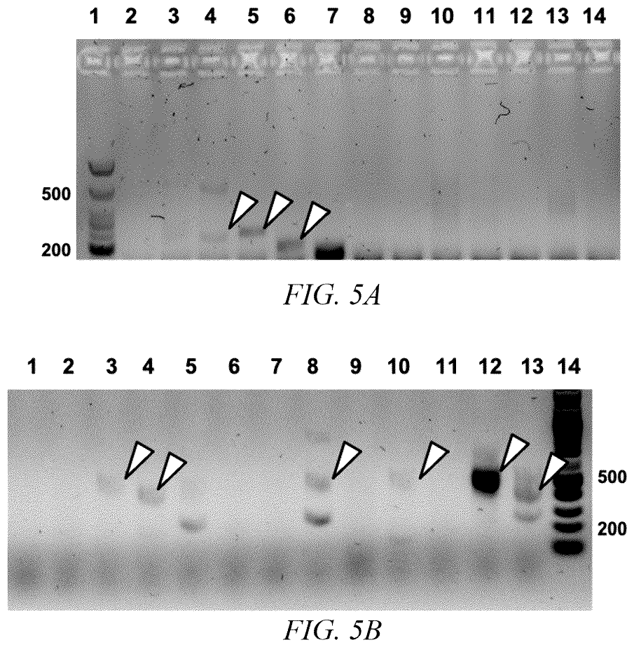

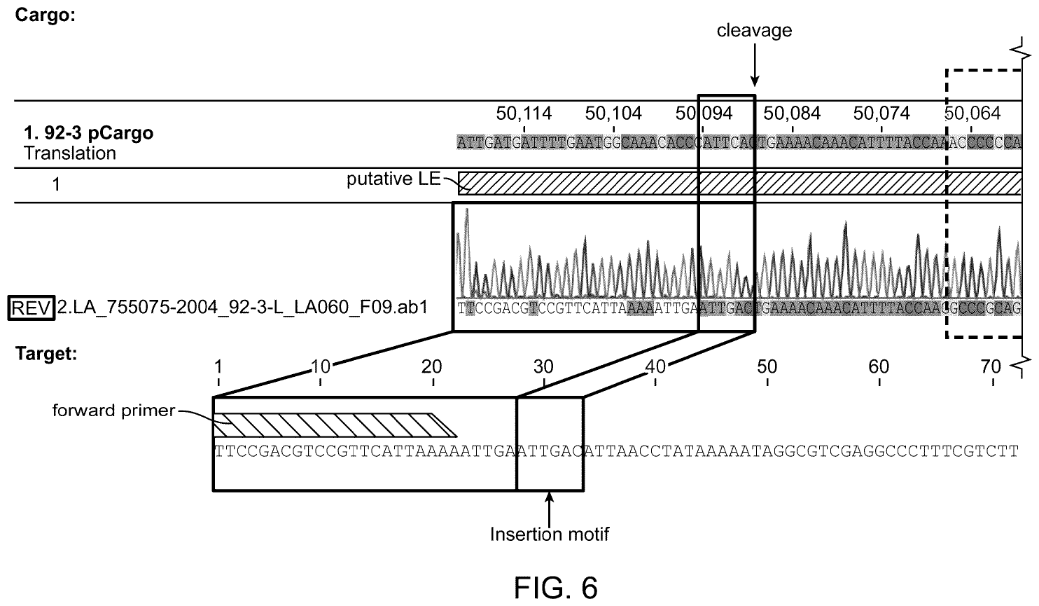

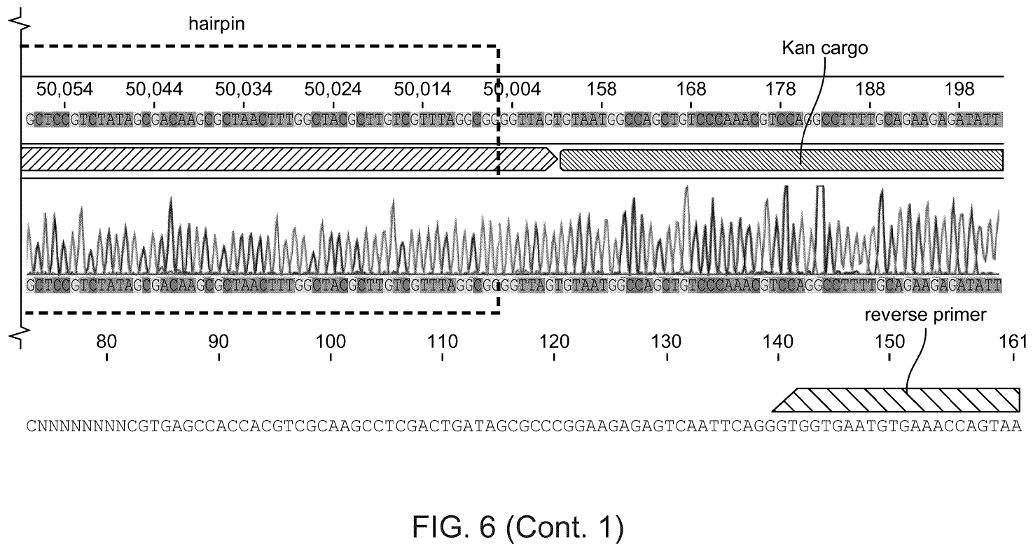

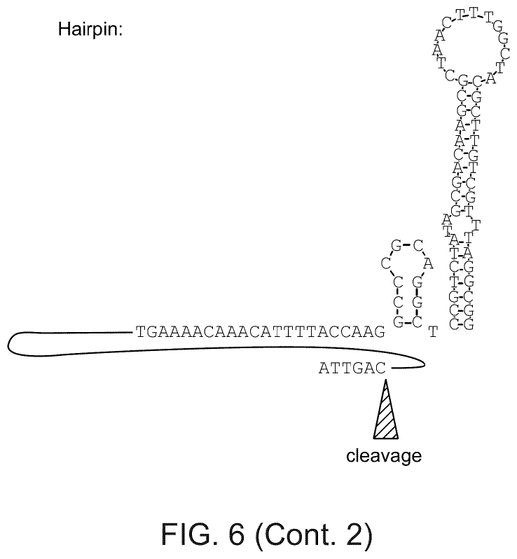

For in vitro activity, TnpA-like protein candidates were first expressed in an in vitro transcription-translation (IVTT) kit following manufacturer's recommended conditions at 37° C. for 2 hours with a minimum template concentration of 8 ng/μL (PURExpress, NEB). Expression was verified via Western blot to the HA tag, with the exception of 92-1, which lacks this tag. (FIG. 4). Transposition assays were set up with 1 μL of IVTT product added per 10 μL reaction, an average of 5 nM of ssDNA cargo and 50 nM of a 161 nt “target” ssDNA containing an 8N randomized sequence in reaction buffer (20 mM HEPES (pH 7.5), 160 mM NaCl, 5 mM MgCl2, 5 mM TCEP, 20 μg/mL BSA, 0.5 μg/mL of poly-dIdC, and 20% glycerol). Control reactions contained a no-template control (NTC) reaction of IVTT where Tris buffer was added instead of PCR template to the IVTT. Reactions were incubated at 37° C. for 1 hour to allow transposition to occur, then the reaction was diluted 10-fold in water and transposition was detected via PCR. The LE junction was detected via a forward primer on the 5′ end of the target and reverse primer within the Kan cargo, and the RE junction via a forward primer in the Kan cargo and a reverse primer on the 3′ end of the target. PCR products were run on an agarose gel to detect transposition (FIGS. 5A and 5B), and sequenced via Sanger and NGS sequencing. Chimeric reads that contained both target and cargo sequence were analyzed to determine the junction of transposition, the insertion motif, and the cleavage sites on the cargo (FIGS. 6-9).

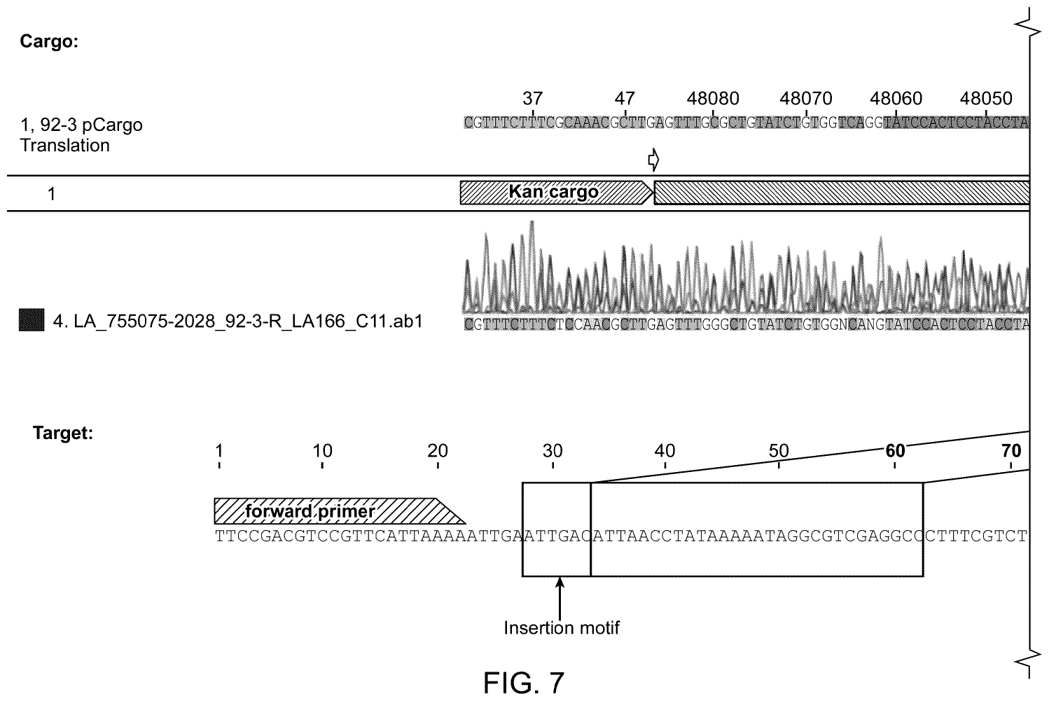

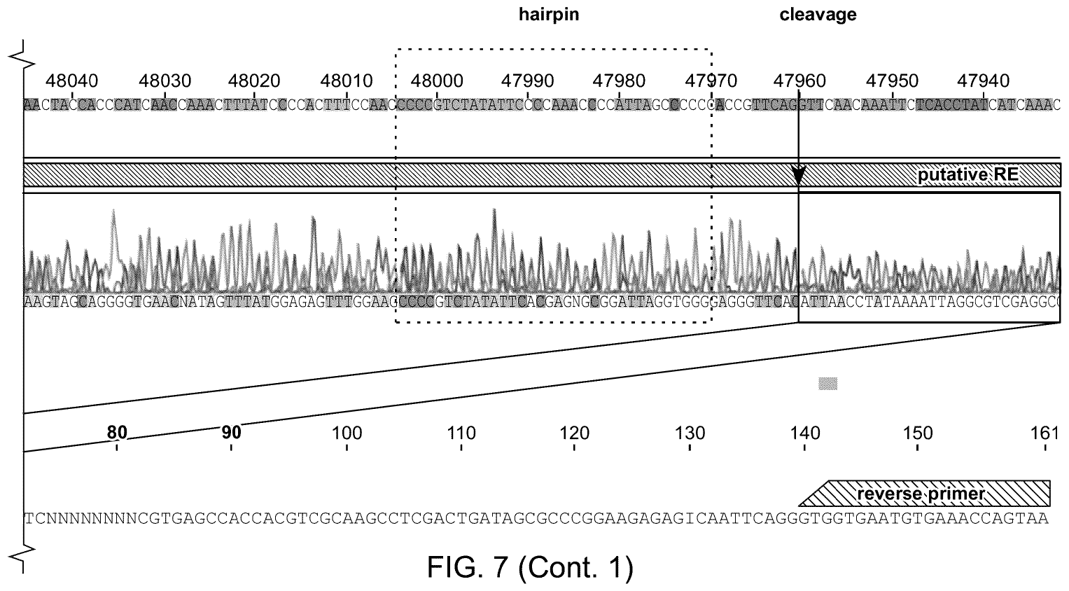

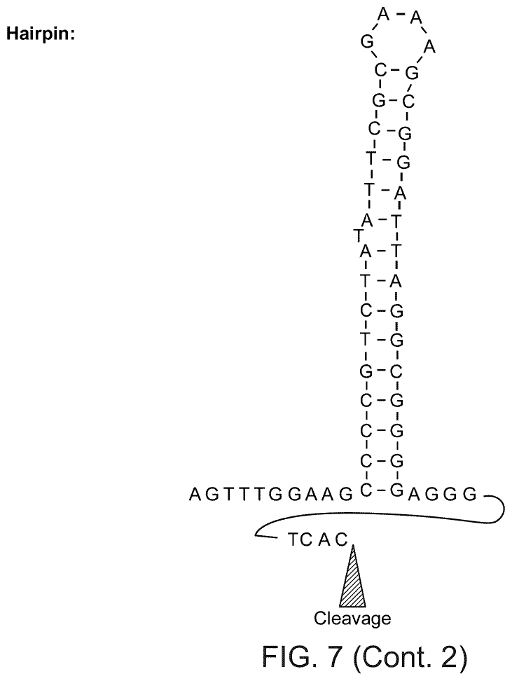

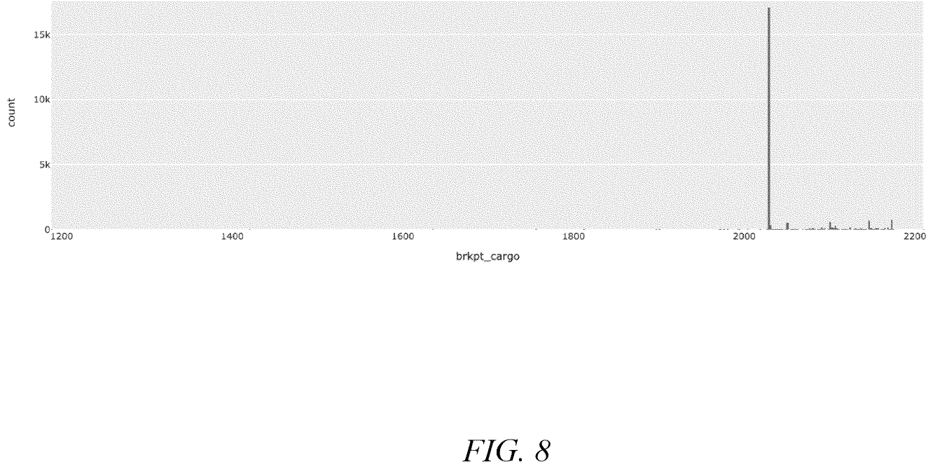

For the LE PCR product, the insertion motif can be identified from overlapping sequence identity between the cargo and the target. For example, the junction between target and the LE for MG92-3 is identified as the point where sequences for the target and cargo no longer overlap (FIG. 6). The insertion motif can be identified via analysis of the flanking sequence of the target DNA without transposition. In the case of insertion into the 8N, the target motif can only be identified without ambiguity in the LE read, not the RE read. For MG92-3, the insertion motif was identified as AATGAC or a subset of nucleotides therein, for example TGAC (FIGS. 6-7). For the RE PCR product, the RE junction is identified via the breakpoint where reads switch between mapping to the cargo and the target (FIG. 7). Sequencing for the LE junction and the RE junction shows the same insertion location. The LE junction was further confirmed via NGS, which identified the same cleavage point in the LE as determined via Sanger sequencing (FIG. 8).

From these data, the LE boundary can be determined as: TGAAAACAAACATTTTACCAAGGCCCGCAGGCTCCGTCTATAGCGACAAGCGCTAAC TTTGGCTACGCTTGTCGTTTAGGCGGGGTTAGT (SEQ ID NO: 475). This is a subset of the full MG92-3 LE and will be recognized by MG92-3 only when flanked by the recognition motif AATGAC, or a subset of nucleotides therein. Similarly, the RE boundary can be identified as: GTTTGCGCTGTATCTGTGGTCAGGTATCCACTCCTACCTAAAGTAGCAGGCATGAAC GAAAGTTTATGCGGAGTTTGGAAGCCCCGTCTATATTCGCGAAAGCGGATTAGGCGG GGAGGGTTCAC (SEQ ID NO: 476), some or all of which is required for recognition, excision, and insertion by TnpA-like proteins. Both of the sequences contain predicted hairpins for TnpA-like protein recognition flanked by non-canonical base pairing interactions which TnpA and TnpA-like proteins recognize (FIGS. 6-7), as described in Cell 132, 208-220 (2008) and Nucleic Acids Res 39, 8503-8512 (2011).

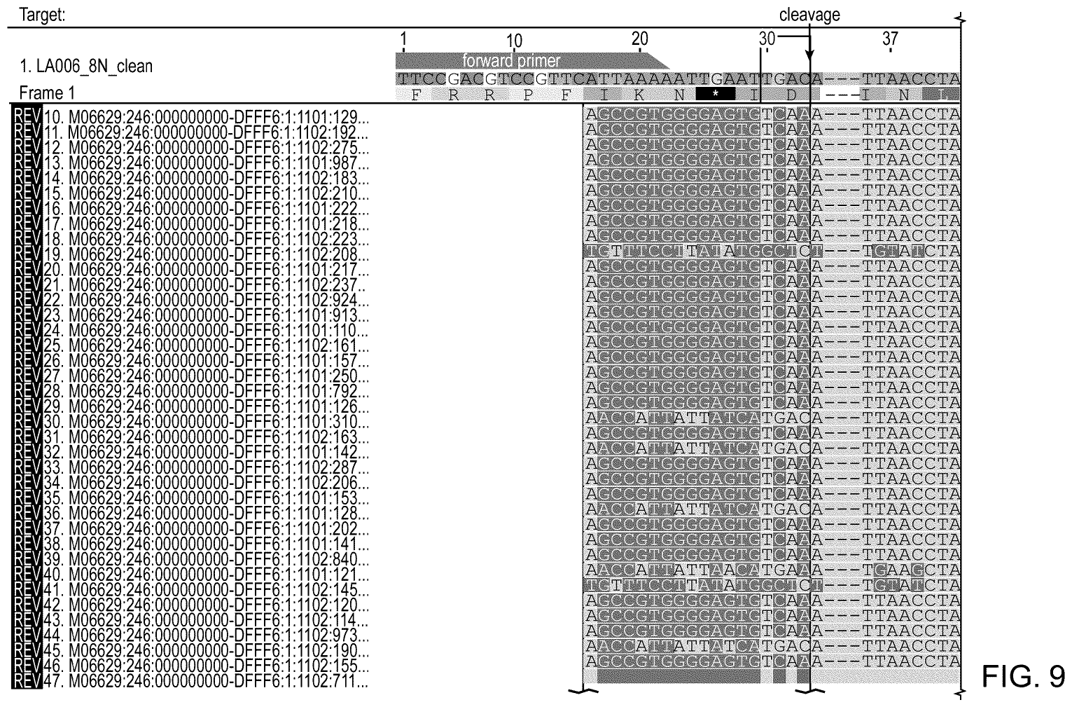

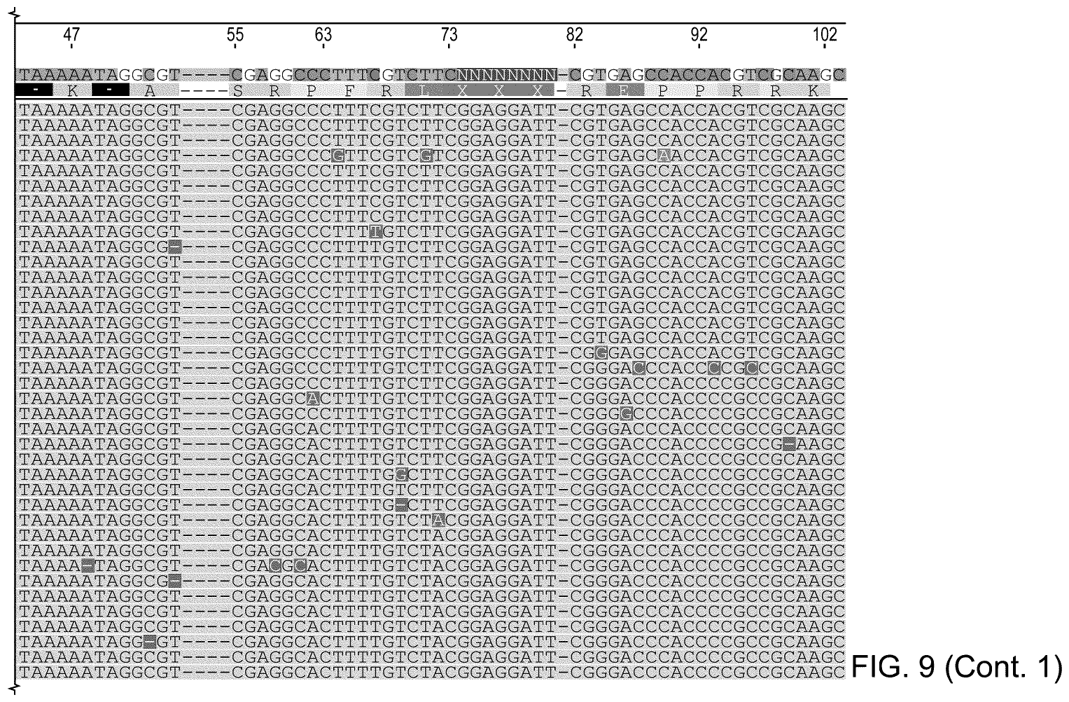

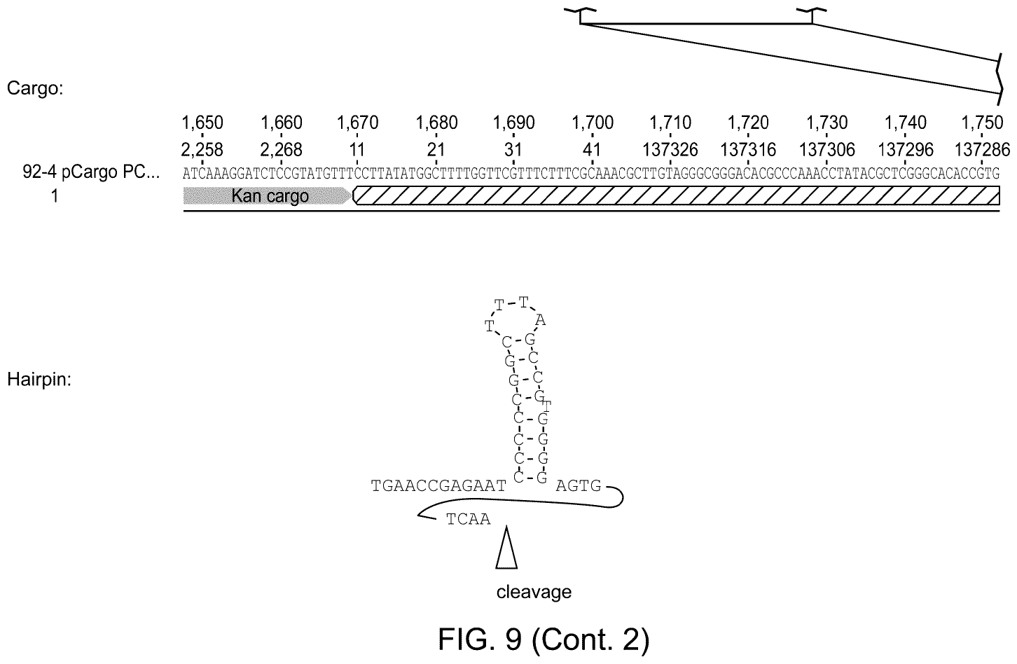

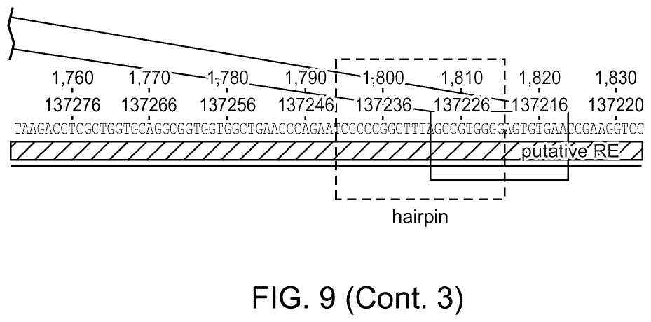

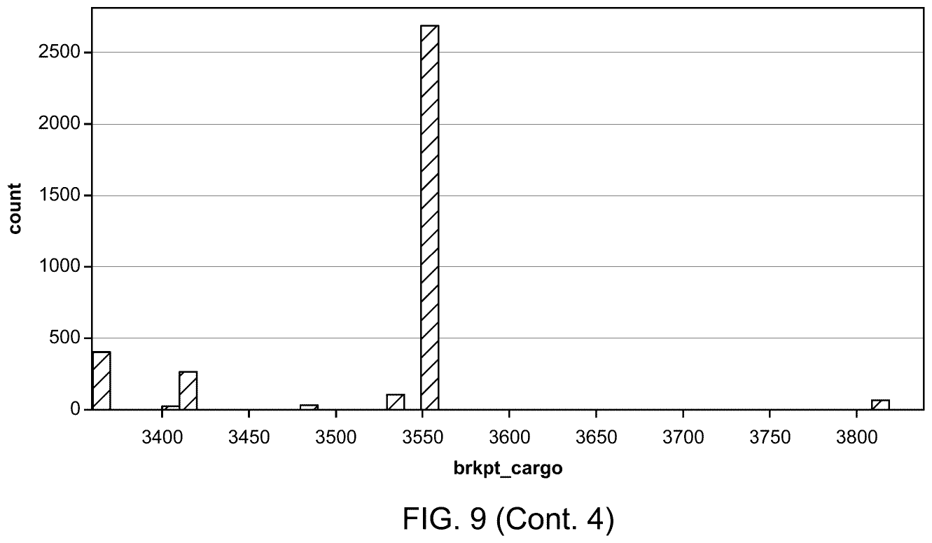

Similarly, activity of MG92-4 was confirmed via NGS detection, with a weaker signal not detectable in Sanger sequencing, showing RE cleavage and insertion (FIG. 9). As this signal was only detectable by NGS, these results suggest that this insertion motif is possible but may not be the optimal insertion sequence.

### Example 12—In Vitro Excision Assay (Prophetic)

To determine in vitro excision activity, TnpA-like protein candidates are expressed in an in vitro transcription-translation (IVTT) kit following manufacturer's recommended conditions at 37° C. for 2 hours with a minimum template concentration of 8 ng/μL (PURExpress, NEB). Excision assays are set with 1 μL of IVTT product added per 10 μL reaction and 100 ng of LE-Kan-RE ssDNA (about 2.2 kb) for 60 minutes at 37° C. in TnpA reaction buffer (20 mM HEPES (pH 7.5), 160 mM NaCl, 5 mM MgCl2, 10 mM TCEP, 20 mg/mL BSA, 0.5 mg of poly-dIdC, and 20% glycerol). Reactions are terminated with the addition of 0.1% SDS and incubation of an additional 15 minutes at 37° C. Reactions are subsequently RNase treated and run on a DNA agarose gel to determine if excision of the LE-Kan-RE ssDNA has occurred. The excised Kan sequence is then gel extracted and submitted for sequencing for determination of the LE and RE cleavage motifs.

### Example 13—In Vivo Excision Assay (Prophetic)

In vivo excision assays are also performed by co-transforming E. coli with 2 plasmids, one containing the LE-Kan-RE cargo and the other TnpA. Following transformation and overnight growth, excision is determined by mini-prep of overnight culture and detection of reclosed donor backbone molecules from which the Kan sequence has been removed on a DNA gel. Controls for this experiment include the transformation of a single plasmid or the transformation of both the TnpA-containing plasmid and the cargo plasmid with an inverted origin of replication. The excised DNA backbone is gel extracted and subjected to sequencing to yield the RE and LE boundaries of the TnpA transposon. The insertion motif remains in the excised backbone and can also be identified at the sealed junction.

### Example 14—Changing Insertion Site Specificity (Prophetic)

Engineering of the insertion recognition site has been demonstrated by Cell 132, 208-220 (2008) without requiring engineering of the TnpA protein. The insertion site recognized by a metagenomics-derived TnpA-like protein described herein is modified via sequence mutations to the insertion site motif and compensatory mutations to the base pairing partners in the LE ssDNA flanking the LE hairpin sequence. A series of single, double, and triple sequence mutations are introduced at rationally designed positions in the insertion site and LE sequence. Recognition and cleavage of the mutated insertion site by wild-type TnpA-like protein is tested concurrently with the wild-type LE insertion sequence using the excision/insertion assays and subsequent sequencing steps described above to compare activity levels.

### Example 15—TnpA can be Used with Sequence-Specific Endonucleases for Programmable Integrations (Prophetic)

IS200/IS605 transposons are a type of mobile genetic element that integrate at specific target sites. These transposons are mobilized by their encoded TnpA-like transposase, an enzyme that belongs to the family of tyrosine (Y) transposases (reviewed in Microbiol Spectr 3, (2015)). The mechanism of IS200/IS605 transposon mobilization involves its excision by TnpA or a TnpA-like protein, followed by its integration at a recognized target site during host replication, when target sites are accessible as ssDNA at the replication fork (Cell 142, 398-408 (2010)).

The RNA-guided binding ability of certain sequence-specific (e.g., Cas) endonuclease effectors to a target site that is shared with TnpA-like proteins may aid TnpA-like effector-mediated integration of a desired cargo by making ssDNA and target site available through formation of the R-loop. Specifically, a desired cargo (for example, a fluorescence marker gene) flanked by TnpA-like-recognizable LE and RE is excised from a donor template by TnpA or a TnpA-like effector and integrated into a desired target site (which contains the TnpA or TnpA-like protein recognizable motif) that is made available by the binding of a (fused) sequence-specific endonuclease. The sequence-specific endonuclease may be engineered to be catalytically dead or have reduced or altered endonuclease (e.g., nickase) activity. Therefore, TnpA-like proteins can be “programmed” to insert a desired cargo into a TAM-dependent target site made available by fused, engineered (e.g., dead or nickase) sequence-specific endonuclease effectors.

### Example 16—In Vitro Testing of TnpA-Like Insertion into R-Loops in dsDNA (Prophetic)

The ability of TnpA-like proteins to insert into ssDNA generated as an R-loop in dsDNA can be tested using active TnpA-like proteins identified in vitro and their corresponding LE and RE sequences. The R-loop can be generated via a sequence-specific endonuclease, such as an RNA-directed nuclease-dead enzyme or nickase that is expressed in an IVTT reaction or added as purified RNP. The TnpA-like protein is tested as described in the in vitro insertion assay, except the target ssDNA is replaced by the dsDNA and RNP. Insertion activity is assayed via PCR with a primer in the dsDNA target and the ssDNA cargo, flanking either the LE junction or the RE junction. The optimal location of the insertion site is tested by placing the insertion motif at various positions along the R-loop to determine the site with best accessibility by the TnpA-like protein. Insertion into ssDNA bubbles in dsDNA where mismatched DNA strands are annealed can also be tested.

TABLE 2
Protein and nucleic acid sequences referred to herein

| Cat. | SEQ ID NO: | Description | Type |
|---|---|---|---|
| MG92 transposition proteins | 1 | MG92-1-A transposition protein | protein |
| MG92 transposition proteins | 2 | MG92-1-B transposition protein | protein |
| MG92 transposition proteins | 3 | MG92-2-A transposition protein | protein |
| MG92 transposition proteins | 4 | MG92-2-B transposition protein | protein |
| MG92 transposition proteins | 5 | MG92-3-A transposition protein | protein |
| MG92 transposition proteins | 6 | MG92-3-B transposition protein | protein |
| MG92 transposition proteins | 7 | MG92-4-A transposition protein | protein |
| MG92 transposition proteins | 8 | MG92-4-B transposition protein | protein |
| MG92 transposition proteins | 9 | MG92-5-A transposition protein | protein |
| MG92 transposition proteins | 10 | MG92-5-B transposition protein | protein |
| MG92 transposition proteins | 11 | MG92-6-A transposition protein | protein |
| MG92 transposition proteins | 12 | MG92-6-B transposition protein | protein |
| MG92 transposition proteins | 13 | MG92-7-A transposition protein | protein |
| MG92 transposition proteins | 14 | MG92-7-B transposition protein | protein |
| MG92 transposition proteins | 15 | MG92-8-A transposition protein | protein |
| MG92 transposition proteins | 16 | MG92-9-A transposition protein | protein |
| MG92 transposition proteins | 17 | MG92-9-B transposition protein | protein |
| MG92 transposition proteins | 18 | MG92-10 transposition protein | protein |
| MG92 transposition proteins | 19 | MG92-11 transposition protein | protein |
| MG92 transposition proteins | 20 | MG92-12 transposition protein | protein |
| MG92 transposition proteins | 21 | MG92-13 transposition protein | protein |
| MG92 transposition proteins | 22 | MG92-14 transposition protein | protein |
| MG92 transposition proteins | 23 | MG92-15 transposition protein | protein |
| MG92 transposition proteins | 24 | MG92-17 transposition protein | protein |
| MG92 transposition proteins | 25 | MG92-19 transposition protein | protein |
| MG92 transposition proteins | 26 | MG92-20 transposition protein | protein |
| MG92 transposition proteins | 27 | MG92-21 transposition protein | protein |
| MG92 transposition proteins | 28 | MG92-22 transposition protein | protein |
| MG92 transposition proteins | 29 | MG92-23 transposition protein | protein |
| MG92 transposition proteins | 30 | MG92-24 transposition protein | protein |
| MG92 transposition proteins | 31 | MG92-25 transposition protein | protein |
| MG92 transposition proteins | 32 | MG92-26 transposition protein | protein |
| MG92 transposition proteins | 33 | MG92-27 transposition protein | protein |
| MG92 transposition proteins | 34 | MG92-28 transposition protein | protein |
| MG92 transposition proteins | 35 | MG92-29 transposition protein | protein |
| MG92 transposition proteins | 36 | MG92-30 transposition protein | protein |
| MG92 transposition proteins | 37 | MG92-31 transposition protein | protein |
| MG92 transposition proteins | 38 | MG92-32 transposition protein | protein |
| MG92 transposition proteins | 39 | MG92-33 transposition protein | protein |
| MG92 transposition proteins | 40 | MG92-34 transposition protein | protein |
| MG92 transposition proteins | 41 | MG92-35 transposition protein | protein |
| MG92 transposition proteins | 42 | MG92-36 transposition protein | protein |
| MG92 transposition proteins | 43 | MG92-37 transposition protein | protein |
| MG92 transposition proteins | 44 | MG92-38 transposition protein | protein |
| MG92 transposition proteins | 45 | MG92-39 transposition protein | protein |
| MG92 transposition proteins | 46 | MG92-40 transposition protein | protein |
| MG92 transposition proteins | 47 | MG92-41 transposition protein | protein |
| MG92 transposition proteins | 48 | MG92-42 transposition protein | protein |
| MG92 transposition proteins | 49 | MG92-43 transposition protein | protein |
| MG92 transposition proteins | 50 | MG92-44 transposition protein | protein |
| MG92 transposition proteins | 51 | MG92-45 transposition protein | protein |
| MG92 transposition proteins | 52 | MG92-46 transposition protein | protein |
| MG92 transposition proteins | 53 | MG92-47 transposition protein | protein |
| MG92 transposition proteins | 54 | MG92-48 transposition protein | protein |
| MG92 transposition proteins | 55 | MG92-49 transposition protein | protein |
| MG92 transposition proteins | 56 | MG92-50 transposition protein | protein |
| MG92 transposition proteins | 57 | MG92-51 transposition protein | protein |
| MG92 transposition proteins | 58 | MG92-52 transposition protein | protein |
| MG92 transposition proteins | 59 | MG92-53 transposition protein | protein |
| MG92 transposition proteins | 50 | MG92-54 transposition protein | protein |
| MG92 transposition proteins | 61 | MG92-55 transposition protein | protein |
| MG92 transposition proteins | 62 | MG92-56 transposition protein | protein |
| MG92 transposition proteins | 63 | MG92-57 transposition protein | protein |
| MG92 transposition proteins | 64 | MG92-58 transposition protein | protein |
| MG92 transposition proteins | 65 | MG92-59 transposition protein | protein |
| MG92 transposition proteins | 66 | MG92-60 transposition protein | protein |
| MG92 transposition proteins | 67 | MG92-61 transposition protein | protein |
| MG92 transposition proteins | 68 | MG92-62 transposition protein | protein |
| MG92 transposition proteins | 69 | MG92-63 transposition protein | protein |
| MG92 transposition proteins | 70 | MG92-64 transposition protein | protein |
| MG92 transposition proteins | 71 | MG92-65 transposition protein | protein |
| MG92 transposition proteins | 72 | MG92-66 transposition protein | protein |
| MG92 transposition proteins | 73 | MG92-67 transposition protein | protein |
| MG92 transposition proteins | 74 | MG92-68 transposition protein | protein |
| MG92 transposition proteins | 75 | MG92-69 transposition protein | protein |
| MG92 transposition proteins | 76 | MG92-70 transposition protein | protein |
| MG92 transposition proteins | 77 | MG92-71 transposition protein | protein |
| MG92 transposition proteins | 78 | MG92-72 transposition protein | protein |
| MG92 transposition proteins | 79 | MG92-73 transposition protein | protein |
| MG92 transposition proteins | 80 | MG92-74 transposition protein | protein |
| MG92 transposition proteins | 81 | MG92-75 transposition protein | protein |
| MG92 transposition proteins | 82 | MG92-76 transposition protein | protein |
| MG92 transposition proteins | 83 | MG92-77 transposition protein | protein |
| MG92 transposition proteins | 84 | MG92-78 transposition protein | protein |
| MG92 transposition proteins | 85 | MG92-79 transposition protein | protein |
| MG92 transposition proteins | 86 | MG92-80 transposition protein | protein |
| MG92 transposition proteins | 87 | MG92-81 transposition protein | protein |
| MG92 transposition proteins | 88 | MG92-82 transposition protein | protein |
| MG92 transposition proteins | 89 | MG92-83 transposition protein | protein |
| MG92 transposition proteins | 90 | MG92-84 transposition protein | protein |
| MG92 transposition proteins | 91 | MG92-85 transposition protein | protein |
| MG92 transposition proteins | 92 | MG92-86 transposition protein | protein |
| MG92 transposition proteins | 93 | MG92-87 transposition protein | protein |
| MG92 transposition proteins | 94 | MG92-88 transposition protein | protein |
| MG92 transposition proteins | 95 | MG92-89 transposition protein | protein |
| MG92 transposition proteins | 96 | MG92-90 transposition protein | protein |
| MG92 transposition proteins | 97 | MG92-91 transposition protein | protein |
| MG92 transposition proteins | 98 | MG92-92 transposition protein | protein |
| MG92 transposition proteins | 99 | MG92-93 transposition protein | protein |
| MG92 transposition proteins | 100 | MG92-94 transposition protein | protein |
| MG92 transposition proteins | 101 | MG92-95 transposition protein | protein |
| MG92 transposition proteins | 102 | MG92-96 transposition protein | protein |
| MG92 transposition proteins | 103 | MG92-97 transposition protein | protein |
| MG92 transposition proteins | 104 | MG92-98 transposition protein | protein |
| MG92 transposition proteins | 105 | MG92-99 transposition protein | protein |
| MG92 transposition proteins | 106 | MG92-100 transposition protein | protein |
| MG92 transposition proteins | 107 | MG92-101 transposition protein | protein |
| MG92 transposition proteins | 108 | MG92-102 transposition protein | protein |
| MG92 transposition proteins | 109 | MG92-103 transposition protein | protein |
| MG92 transposition proteins | 110 | MG92-104 transposition protein | protein |
| MG92 transposition proteins | 111 | MG92-105 transposition protein | protein |
| MG92 transposition proteins | 112 | MG92-106 transposition protein | protein |
| MG92 transposition proteins | 113 | MG92-107 transposition protein | protein |
| MG92 transposition proteins | 114 | MG92-108 transposition protein | protein |
| MG92 transposition proteins | 115 | MG92-109 transposition protein | protein |
| MG92 transposition proteins | 116 | MG92-110 transposition protein | protein |
| MG92 transposition proteins | 117 | MG92-111 transposition protein | protein |
| MG92 transposition proteins | 118 | MG92-112 transposition protein | protein |
| MG92 transposition proteins | 119 | MG92-113 transposition protein | protein |
| MG92 transposition proteins | 120 | MG92-114 transposition protein | protein |
| MG92 transposition proteins | 121 | MG92-115 transposition protein | protein |
| MG92 transposition proteins | 122 | MG92-116 transposition protein | protein |
| MG92 transposition proteins | 123 | MG92-117 transposition protein | protein |
| MG92 transposition proteins | 124 | MG92-118 transposition protein | protein |
| MG92 transposition proteins | 125 | MG92-119 transposition protein | protein |
| MG92 transposition proteins | 126 | MG92-120 transposition protein | protein |
| MG92 transposition proteins | 127 | MG92-121 transposition protein | protein |
| MG92 transposition proteins | 128 | MG92-122 transposition protein | protein |
| MG92 transposition proteins | 129 | MG92-123 transposition protein | protein |
| MG92 transposition proteins | 130 | MG92-124 transposition protein | protein |
| MG92 transposition proteins | 131 | MG92-125 transposition protein | protein |
| MG92 transposition proteins | 132 | MG92-126 transposition protein | protein |
| MG92 transposition proteins | 133 | MG92-127 transposition protein | protein |
| MG92 transposition proteins | 134 | MG92-128 transposition protein | protein |
| MG92 transposition proteins | 135 | MG92-129 transposition protein | protein |
| MG92 transposition proteins | 136 | MG92-130 transposition protein | protein |
| MG92 transposition proteins | 137 | MG92-131 transposition protein | protein |
| MG92 transposition proteins | 138 | MG92-132 transposition protein | protein |
| MG92 transposition proteins | 139 | MG92-133 transposition protein | protein |
| MG92 transposition proteins | 140 | MG92-134 transposition protein | protein |
| MG92 transposition proteins | 141 | MG92-135 transposition protein | protein |
| MG92 transposition proteins | 142 | MG92-136 transposition protein | protein |
| MG92 transposition proteins | 143 | MG92-137 transposition protein | protein |
| MG92 transposition proteins | 144 | MG92-138 transposition protein | protein |
| MG92 transposition proteins | 145 | MG92-139 transposition protein | protein |
| MG92 transposition proteins | 146 | MG92-140 transposition protein | protein |
| MG92 transposition proteins | 147 | MG92-141 transposition protein | protein |
| MG92 transposition proteins | 148 | MG92-142 transposition protein | protein |
| MG92 transposition proteins | 149 | MG92-143 transposition protein | protein |
| MG92 transposition proteins | 150 | MG92-144 transposition protein | protein |
| MG92 transposition proteins | 151 | MG92-145 transposition protein | protein |
| MG92 transposition proteins | 152 | MG92-146 transposition protein | protein |
| MG92 transposition proteins | 153 | MG92-147 transposition protein | protein |
| MG92 transposition proteins | 154 | MG92-148 transposition protein | protein |
| MG92 transposition proteins | 155 | MG92-149 transposition protein | protein |
| MG92 transposition proteins | 156 | MG92-150 transposition protein | protein |
| MG92 transposition proteins | 157 | MG92-151 transposition protein | protein |
| MG92 transposition proteins | 158 | MG92-152 transposition protein | protein |
| MG92 transposition proteins | 159 | MG92-153 transposition protein | protein |
| MG92 transposition proteins | 160 | MG92-154 transposition protein | protein |
| MG92 transposition proteins | 161 | MG92-155 transposition protein | protein |
| MG92 transposition proteins | 162 | MG92-156 transposition protein | protein |
| MG92 transposition proteins | 163 | MG92-157 transposition protein | protein |
| MG92 transposition proteins | 164 | MG92-158 transposition protein | protein |
| MG92 transposition proteins | 165 | MG92-159 transposition protein | protein |
| MG92 transposition proteins | 166 | MG92-160 transposition protein | protein |
| MG92 transposition proteins | 167 | MG92-161 transposition protein | protein |
| MG92 transposition proteins | 168 | MG92-162 transposition protein | protein |
| MG92 transposition proteins | 169 | MG92-163 transposition protein | protein |
| MG92 transposition proteins | 170 | MG92-164 transposition protein | protein |
| MG92 transposition proteins | 171 | MG92-165 transposition protein | protein |
| MG92 transposition proteins | 172 | MG92-166 transposition protein | protein |
| MG92 transposition proteins | 173 | MG92-167 transposition protein | protein |
| MG92 transposition proteins | 174 | MG92-168 transposition protein | protein |
| MG92 transposition proteins | 175 | MG92-169 transposition protein | protein |
| MG92 transposition proteins | 176 | MG92-170 transposition protein | protein |
| MG92 transposition proteins | 177 | MG92-171 transposition protein | protein |
| MG92 transposition proteins | 178 | MG92-172 transposition protein | protein |
| MG92 transposition proteins | 179 | MG92-173 transposition protein | protein |
| MG92 transposition proteins | 180 | MG92-174 transposition protein | protein |
| MG92 transposition proteins | 181 | MG92-175 transposition protein | protein |
| MG92 transposition proteins | 182 | MG92-176 transposition protein | protein |
| MG92 transposition proteins | 183 | MG92-177 transposition protein | protein |
| MG92 transposition proteins | 184 | MG92-178 transposition protein | protein |
| MG92 transposition proteins | 185 | MG92-179 transposition protein | protein |
| MG92 transposition proteins | 186 | MG92-180 transposition protein | protein |
| MG92 transposition proteins | 187 | MG92-181 transposition protein | protein |
| MG92 transposition proteins | 188 | MG92-182 transposition protein | protein |
| MG92 transposition proteins | 189 | MG92-183 transposition protein | protein |
| MG92 transposition proteins | 190 | MG92-184 transposition protein | protein |
| MG92 transposition proteins | 191 | MG92-185 transposition protein | protein |
| MG92 transposition proteins | 192 | MG92-186 transposition protein | protein |
| MG92 transposition proteins | 193 | MG92-187 transposition protein | protein |
| MG92 transposition proteins | 194 | MG92-188 transposition protein | protein |
| MG92 transposition proteins | 195 | MG92-189 transposition protein | protein |
| MG92 transposition proteins | 196 | MG92-190 transposition protein | protein |
| MG92 transposition proteins | 197 | MG92-191 transposition protein | protein |
| MG92 transposition proteins | 198 | MG92-192 transposition protein | protein |
| MG92 transposition proteins | 199 | MG92-193 transposition protein | protein |
| MG92 transposition proteins | 200 | MG92-194 transposition protein | protein |
| MG92 transposition proteins | 201 | MG92-195 transposition protein | protein |
| MG92 transposition proteins | 202 | MG92-196 transposition protein | protein |
| MG92 transposition proteins | 203 | MG92-197 transposition protein | protein |
| MG92 transposition proteins | 204 | MG92-198 transposition protein | protein |
| MG92 transposition proteins | 205 | MG92-199 transposition protein | protein |
| MG92 transposition proteins | 206 | MG92-200 transposition protein | protein |
| MG92 transposition proteins | 207 | MG92-201 transposition protein | protein |
| MG92 transposition proteins | 208 | MG92-202 transposition protein | protein |
| MG92 transposition proteins | 209 | MG92-203 transposition protein | protein |
| MG92 transposition proteins | 210 | MG92-204 transposition protein | protein |
| MG92 transposition proteins | 211 | MG92-205 transposition protein | protein |
| MG92 transposition proteins | 212 | MG92-206 transposition protein | protein |
| MG92 transposition proteins | 213 | MG92-207 transposition protein | protein |
| MG92 transposition proteins | 214 | MG92-208 transposition protein | protein |
| MG92 transposition proteins | 215 | MG92-209 transposition protein | protein |
| MG92 transposition proteins | 216 | MG92-210 transposition protein | protein |
| MG92 transposition proteins | 217 | MG92-211 transposition protein | protein |
| MG92 transposition proteins | 218 | MG92-212 transposition protein | protein |
| MG92 transposition proteins | 219 | MG92-213 transposition protein | protein |
| MG92 transposition proteins | 220 | MG92-214 transposition protein | protein |
| MG92 transposition proteins | 221 | MG92-215 transposition protein | protein |
| MG92 transposition proteins | 222 | MG92-216 transposition protein | protein |
| MG92 transposition proteins | 223 | MG92-217 transposition protein | protein |
| MG92 transposition proteins | 224 | MG92-218 transposition protein | protein |
| MG92 transposition proteins | 225 | MG92-219 transposition protein | protein |
| MG92 transposition proteins | 226 | MG92-220 transposition protein | protein |
| MG92 transposition proteins | 227 | MG92-221 transposition protein | protein |
| MG92 transposition proteins | 228 | MG92-222 transposition protein | protein |
| MG92 transposition proteins | 229 | MG92-223 transposition protein | protein |
| MG92 transposition proteins | 230 | MG92-224 transposition protein | protein |
| MG92 transposition proteins | 231 | MG92-225 transposition protein | protein |
| MG92 transposition proteins | 232 | MG92-226 transposition protein | protein |
| MG92 transposition proteins | 233 | MG92-227 transposition protein | protein |
| MG92 transposition proteins | 234 | MG92-228 transposition protein | protein |
| MG92 transposition proteins | 235 | MG92-229 transposition protein | protein |
| MG92 transposition proteins | 236 | MG92-230 transposition protein | protein |
| MG92 transposition proteins | 237 | MG92-231 transposition protein | protein |
| MG92 transposition proteins | 238 | MG92-232 transposition protein | protein |
| MG92 transposition proteins | 239 | MG92-233 transposition protein | protein |
| MG92 transposition proteins | 240 | MG92-234 transposition protein | protein |
| MG92 transposition proteins | 241 | MG92-235 transposition protein | protein |
| MG92 transposition proteins | 242 | MG92-236 transposition protein | protein |
| MG92 transposition proteins | 243 | MG92-237 transposition protein | protein |
| MG92 transposition proteins | 244 | MG92-238 transposition protein | protein |
| MG92 transposition proteins | 245 | MG92-239 transposition protein | protein |
| MG92 transposition proteins | 246 | MG92-240 transposition protein | protein |
| MG92 transposition proteins | 247 | MG92-241 transposition protein | protein |
| MG92 transposition proteins | 248 | MG92-242 transposition protein | protein |
| MG92 transposition proteins | 249 | MG92-243 transposition protein | protein |
| MG92 transposition proteins | 250 | MG92-244 transposition protein | protein |
| MG92 transposition proteins | 251 | MG92-245 transposition protein | protein |
| MG92 transposition proteins | 252 | MG92-246 transposition protein | protein |
| MG92 transposition proteins | 253 | MG92-247 transposition protein | protein |
| MG92 transposition proteins | 254 | MG92-248 transposition protein | protein |
| MG92 transposition proteins | 255 | MG92-249 transposition protein | protein |
| MG92 transposition proteins | 256 | MG92-250 transposition protein | protein |
| MG92 transposition proteins | 257 | MG92-251 transposition protein | protein |
| MG92 transposition proteins | 258 | MG92-252 transposition protein | protein |
| MG92 transposition proteins | 259 | MG92-253 transposition protein | protein |
| MG92 transposition proteins | 260 | MG92-254 transposition protein | protein |
| MG92 transposition proteins | 261 | MG92-255 transposition protein | protein |
| MG92 transposition proteins | 262 | MG92-256 transposition protein | protein |
| MG92 transposition proteins | 263 | MG92-257 transposition protein | protein |
| MG92 transposition proteins | 264 | MG92-258 transposition protein | protein |
| MG92 transposition proteins | 265 | MG92-259 transposition protein | protein |
| MG92 transposition proteins | 266 | MG92-260 transposition protein | protein |
| MG92 transposition proteins | 267 | MG92-261 transposition protein | protein |
| MG92 transposition proteins | 268 | MG92-262 transposition protein | protein |
| MG92 transposition proteins | 269 | MG92-263 transposition protein | protein |
| MG92 transposition proteins | 270 | MG92-264 transposition protein | protein |
| MG92 transposition proteins | 271 | MG92-265 transposition protein | protein |
| MG92 transposition proteins | 272 | MG92-266 transposition protein | protein |
| MG92 transposition proteins | 273 | MG92-267 transposition protein | protein |
| MG92 transposition proteins | 274 | MG92-268 transposition protein | protein |
| MG92 transposition proteins | 275 | MG92-269 transposition protein | protein |
| MG92 transposition proteins | 276 | MG92-270 transposition protein | protein |
| MG92 transposition proteins | 277 | MG92-271 transposition protein | protein |
| MG92 transposition proteins | 278 | MG92-272 transposition protein | protein |
| MG92 transposition proteins | 279 | MG92-273 transposition protein | protein |
| MG92 transposition proteins | 280 | MG92-274 transposition protein | protein |
| MG92 transposition proteins | 281 | MG92-275 transposition protein | protein |
| MG92 transposition proteins | 282 | MG92-276 transposition protein | protein |
| MG92 transposition proteins | 283 | MG92-278 transposition protein | protein |
| MG92 transposition proteins | 284 | MG92-279 transposition protein | protein |
| MG92 transposition proteins | 285 | MG92-280 transposition protein | protein |
| MG92 transposition proteins | 286 | MG92-281 transposition protein | protein |
| MG92 transposition proteins | 287 | MG92-282 transposition protein | protein |
| MG92 transposition proteins | 288 | MG92-283 transposition protein | protein |
| MG92 transposition proteins | 289 | MG92-284 transposition protein | protein |
| MG92 transposition proteins | 290 | MG92-285 transposition protein | protein |
| MG92 transposition proteins | 291 | MG92-286 transposition protein | protein |
| MG92 transposition proteins | 292 | MG92-287 transposition protein | protein |
| MG92 transposition proteins | 293 | MG92-288 transposition protein | protein |
| MG92 transposition proteins | 294 | MG92-290 transposition protein | protein |
| MG92 transposition proteins | 295 | MG92-291 transposition protein | protein |
| MG92 transposition proteins | 296 | MG92-292 transposition protein | protein |
| MG92 transposition proteins | 297 | MG92-293 transposition protein | protein |
| MG92 transposition proteins | 298 | MG92-294 transposition protein | protein |
| MG92 transposition proteins | 299 | MG92-295 transposition protein | protein |
| MG92 transposition proteins | 300 | MG92-296 transposition protein | protein |
| MG92 transposition proteins | 301 | MG92-297 transposition protein | protein |
| MG92 transposition proteins | 302 | MG92-298 transposition protein | protein |
| MG92 transposition proteins | 303 | MG92-299 transposition protein | protein |
| MG92 transposition proteins | 304 | MG92-300 transposition protein | protein |
| MG92 transposition proteins | 305 | MG92-301 transposition protein | protein |
| MG92 transposition proteins | 306 | MG92-302 transposition protein | protein |
| MG92 transposition proteins | 307 | MG92-303 transposition protein | protein |
| MG92 transposition proteins | 308 | MG92-304 transposition protein | protein |
| MG92 transposition proteins | 309 | MG92-305 transposition protein | protein |
| MG92 transposition proteins | 310 | MG92-306 transposition protein | protein |
| MG92 transposition proteins | 311 | MG92-307 transposition protein | protein |
| MG92 transposition proteins | 312 | MG92-308 transposition protein | protein |
| MG92 transposition proteins | 313 | MG92-309 transposition protein | protein |
| MG92 transposition proteins | 314 | MG92-310 transposition protein | protein |
| MG92 transposition proteins | 315 | MG92-311 transposition protein | protein |
| MG92 transposition proteins | 316 | MG92-312 transposition protein | protein |
| MG92 transposition proteins | 317 | MG92-313 transposition protein | protein |
| MG92 transposition proteins | 318 | MG92-314 transposition protein | protein |
| MG92 transposition proteins | 319 | MG92-315 transposition protein | protein |
| MG92 transposition proteins | 320 | MG92-316 transposition protein | protein |
| MG92 transposition proteins | 321 | MG92-317 transposition protein | protein |
| MG92 transposition proteins | 322 | MG92-318 transposition protein | protein |
| MG92 transposition proteins | 323 | MG92-319 transposition protein | protein |
| MG92 transposition proteins | 324 | MG92-320 transposition protein | protein |
| MG92 transposition proteins | 325 | MG92-321 transposition protein | protein |
| MG92 transposition proteins | 326 | MG92-322 transposition protein | protein |
| MG92 transposition proteins | 327 | MG92-323 transposition protein | protein |
| MG92 transposition proteins | 328 | MG92-324 transposition protein | protein |
| MG92 transposition proteins | 329 | MG92-325 transposition protein | protein |
| MG92 transposition proteins | 330 | MG92-326 transposition protein | protein |
| MG92 transposition proteins | 331 | MG92-327 transposition protein | protein |
| MG92 transposition proteins | 332 | MG92-328 transposition protein | protein |
| MG92 transposition proteins | 333 | MG92-330 transposition protein | protein |
| MG92 transposition proteins | 334 | MG92-332 transposition protein | protein |
| MG92 transposition proteins | 335 | MG92-334 transposition protein | protein |
| MG92 transposition proteins | 336 | MG92-336 transposition protein | protein |
| MG92 transposition proteins | 337 | MG92-338 transposition protein | protein |
| MG92 transposition proteins | 338 | MG92-340 transposition protein | protein |
| MG92 transposition proteins | 339 | MG92-341 transposition protein | protein |
| MG92 transposition proteins | 340 | MG92-342 transposition protein | protein |
| MG92 transposition proteins | 341 | MG92-343 transposition protein | protein |
| MG92 transposition proteins | 342 | MG92-344 transposition protein | protein |
| MG92 transposition proteins | 343 | MG92-345 transposition protein | protein |
| MG92 transposition proteins | 344 | MG92-346 transposition protein | protein |
| MG92 transposition proteins | 345 | MG92-347 transposition protein | protein |
| MG92 transposition proteins | 346 | MG92-348 transposition protein | protein |
| MG92 transposition proteins | 347 | MG92-349 transposition protein | protein |
| MG92 transposition proteins | 348 | MG92-350 transposition protein | protein |
| MG92 transposition proteins | 349 | MG92-351 transposition protein | protein |
| MG92 transposon ends | 350 | MG92-1-A transposon left end (LE) | nucleotide |
| MG92 transposon ends | 351 | MG92-1-A transposon right end (RE) | nucleotide |
| MG92 transposon ends | 352 | MG92-2-A transposon left end (LE) | nucleotide |
| MG92 transposon ends | 353 | MG92-2-A transposon right end (RE) | nucleotide |
| MG92 transposon ends | 354 | MG92-3-A transposon right end (RE) | nucleotide |
| MG92 transposon ends | 355 | MG92-3-A transposon left end (LE) | nucleotide |
| MG92 transposon ends | 356 | MG92-4-A transposon left end (LE) | nucleotide |
| MG92 transposon ends | 357 | MG92-4-A transposon right end (RE) | nucleotide |
| MG92 transposon ends | 358 | MG92-5-A transposon right end (RE) | nucleotide |
| MG92 transposon ends | 359 | MG92-5-A transposon left end (LE) | nucleotide |
| MG92 transposon ends | 360 | MG92-6-A transposon right end (RE) | nucleotide |
| MG92 transposon ends | 361 | MG92-6-A transposon left end (LE) | nucleotide |
| MG92 transposon ends | 362 | MG92-7-A transposon left end (LE) | nucleotide |
| MG92 transposon ends | 363 | MG92-7-A transposon right end (RE) | nucleotide |
| MG92 transposon ends | 364 | MG92-9-A transposon left end (LE) | nucleotide |
| MG92 transposon ends | 365 | MG92-9-A transposon right end (RE) | nucleotide |
| MG92 transposon ends | 366 | MG92-11 transposon right end (RE) | nucleotide |
| MG92 transposon ends | 367 | MG92-11 transposon left end (LE) | nucleotide |
| MG92 transposon ends | 368 | MG92-17 transposon left end (LE) | nucleotide |
| MG92 transposon ends | 369 | MG92-17 transposon right end (RE) | nucleotide |
| MG92 transposon ends | 370 | MG92-20 transposon left end (LE) | nucleotide |
| MG92 transposon ends | 371 | MG92-20 transposon right end (RE) | nucleotide |
| MG92 transposon ends | 372 | MG92-21 transposon right end (RE) | nucleotide |
| MG92 transposon ends | 373 | MG92-21 transposon left end (LE) | nucleotide |
| MG92 transposon ends | 374 | MG92-27 transposon left end (LE) | nucleotide |
| MG92 transposon ends | 375 | MG92-27 transposon right end (RE) | nucleotide |
| MG92 transposon ends | 376 | MG92-28 transposon right end (RE) | nucleotide |
| MG92 transposon ends | 377 | MG92-28 transposon left end (LE) | nucleotide |
| MG92 transposon ends | 378 | MG92-37 transposon left end (LE) | nucleotide |
| MG92 transposon ends | 379 | MG92-37 transposon right end (RE) | nucleotide |
| MG92 transposon ends | 380 | MG92-86 transposon left end (LE) | nucleotide |
| MG92 transposon ends | 381 | MG92-86 transposon right end (RE) | nucleotide |
| MG92 transposon ends | 382 | MG92-136 transposon right end (RE) | nucleotide |
| MG92 transposon ends | 383 | MG92-136 transposon left end (LE) | nucleotide |
| MG92 transposon ends | 384 | MG92-138 transposon right end (RE) | nucleotide |
| MG92 transposon ends | 385 | MG92-138 transposon left end (LE) | nucleotide |
| MG92 transposon ends | 386 | MG92-155, MG92-160 transposon left end (LE) | nucleotide |
| MG92 transposon ends | 387 | MG92-155, MG92-160 transposon right end | nucleotide |
| | | (RE) | |
| MG92 transposon ends | 388 | MG92-157 transposon right end (RE) | nucleotide |
| MG92 transposon ends | 389 | MG92-157 transposon left end (LE) | nucleotide |
| MG92 transposon ends | 390 | MG92-159 transposon right end (RE) | nucleotide |
| MG92 transposon ends | 391 | MG92-159 transposon left end (LE) | nucleotide |
| MG92 transposon ends | 392 | MG92-162 transposon right end (RE) | nucleotide |
| MG92 transposon ends | 393 | MG92-162 transposon left end (LE) | nucleotide |
| MG92 transposon ends | 394 | MG92-163 transposon left end (LE) | nucleotide |
| MG92 transposon ends | 395 | MG92-163 transposon right end (RE) | nucleotide |
| MG92 transposon ends | 396 | MG92-164 transposon right end (RE) | nucleotide |
| MG92 transposon ends | 397 | MG92-164 transposon left end (LE) | nucleotide |
| MG92 transposon ends | 398 | MG92-165 transposon right end (RE) | nucleotide |
| MG92 transposon ends | 399 | MG92-165 transposon left end (LE) | nucleotide |
| MG92 transposon ends | 400 | MG92-172 transposon left end (LE) | nucleotide |
| MG92 transposon ends | 401 | MG92-172 transposon right end (RE) | nucleotide |
| MG92 transposon ends | 402 | MG92-174 transposon right end (RE) | nucleotide |
| MG92 transposon ends | 403 | MG92-174 transposon left end (LE) | nucleotide |
| MG92 transposon ends | 404 | MG92-177 transposon left end (LE) | nucleotide |
| MG92 transposon ends | 405 | MG92-177 transposon right end (RE) | nucleotide |
| MG92 transposon ends | 406 | MG92-183 transposon left end (LE) | nucleotide |
| MG92 transposon ends | 407 | MG92-183 transposon right end (RE) | nucleotide |
| MG92 transposon ends | 408 | MG92-185 transposon left end (LE) | nucleotide |
| MG92 transposon ends | 409 | MG92-185 transposon right end (RE) | nucleotide |
| MG92 transposon ends | 410 | MG92-187 transposon left end (LE) | nucleotide |
| MG92 transposon ends | 411 | MG92-187 transposon right end (RE) | nucleotide |
| MG92 transposon ends | 412 | MG92-188 transposon left end (LE) | nucleotide |
| MG92 transposon ends | 413 | MG92-188 transposon right end (RE) | nucleotide |
| MG92 transposon ends | 414 | MG92-189 transposon left end (LE) | nucleotide |
| MG92 transposon ends | 415 | MG92-189 transposon right end (RE) | nucleotide |
| MG92 transposon ends | 416 | MG92-196 transposon left end (LE) | nucleotide |
| MG92 transposon ends | 417 | MG92-196 transposon right end (RE) | nucleotide |
| MG92 transposon ends | 418 | MG92-222 transposon left end (LE) | nucleotide |
| MG92 transposon ends | 419 | MG92-222, MG92-266 transposon right end | nucleotide |
| | | (RE) | |
| MG92 transposon ends | 420 | MG92-224 transposon right end (RE) | nucleotide |
| MG92 transposon ends | 421 | MG92-224 transposon left end (LE) | nucleotide |
| MG92 transposon ends | 422 | MG92-226 transposon right end (RE) | nucleotide |
| MG92 transposon ends | 423 | MG92-226 transposon left end (LE) | nucleotide |
| MG92 transposon ends | 424 | MG92-264 transposon left end (LE) | nucleotide |
| MG92 transposon ends | 425 | MG92-264 transposon right end (RE) | nucleotide |
| MG92 transposon ends | 426 | MG92-266 transposon left end (LE) | nucleotide |
| MG92 transposon ends | 427 | MG92-267 transposon right end (RE) | nucleotide |
| MG92 transposon ends | 428 | MG92-267 transposon left end (LE) | nucleotide |
| MG92 transposon ends | 429 | MG92-272 transposon right end (RE) | nucleotide |
| MG92 transposon ends | 430 | MG92-272 transposon left end (LE) | nucleotide |
| MG92 transposon ends | 431 | MG92-274 transposon right end (RE) | nucleotide |
| MG92 transposon ends | 432 | MG92-274 transposon left end (LE) | nucleotide |
| MG92 transposon ends | 433 | MG92-284 transposon left end (LE) | nucleotide |
| MG92 transposon ends | 434 | MG92-284 transposon right end (RE) | nucleotide |
| MG92 transposon ends | 435 | MG92-288 transposon left end (LE) | nucleotide |
| MG92 transposon ends | 436 | MG92-288 transposon right end (RE) | nucleotide |
| MG92 transposon ends | 437 | MG92-291 transposon left end (LE) | nucleotide |
| MG92 transposon ends | 438 | MG92-291 transposon right end (RE) | nucleotide |
| MG92 transposon ends | 439 | MG92-295 transposon right end (RE) | nucleotide |
| MG92 transposon ends | 440 | MG92-295 transposon left end (LE) | nucleotide |
| MG92 transposon ends | 441 | MG92-302 transposon right end (RE) | nucleotide |
| MG92 transposon ends | 442 | MG92-302 transposon left end (LE) | nucleotide |
| MG92 transposon ends | 443 | MG92-310 transposon right end (RE) | nucleotide |
| MG92 transposon ends | 444 | MG92-310 transposon left end (LE) | nucleotide |
| MG92 transposon ends | 445 | MG92-311 transposon left end (LE) | nucleotide |
| MG92 transposon ends | 446 | MG92-311 transposon right end (RE) | nucleotide |
| MG92 transposon ends | 447 | MG92-312 transposon right end (RE) | nucleotide |
| MG92 transposon ends | 448 | MG92-312 transposon left end (LE) | nucleotide |
| MG92 transposon ends | 449 | MG92-322 transposon left end (LE) | nucleotide |
| MG92 transposon ends | 450 | MG92-322 transposon right end (RE) | nucleotide |
| MG92 transposon ends | 451 | MG92-323 transposon left end (LE) | nucleotide |
| MG92 transposon ends | 452 | MG92-323 transposon right end (RE) | nucleotide |
| MG92 transposon ends | 453 | MG92-344 transposon left end (LE) | nucleotide |
| MG92 transposon ends | 454 | MG92-344 transposon right end (RE) | nucleotide |

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1.-156. (canceled)

157. An engineered transposase system, comprising:
(a) a double-stranded nucleic acid comprising a heterologous cargo nucleotide sequence, wherein said heterologous cargo nucleotide sequence is configured to interact with a transposase; and
(b) said transposase, wherein:
(i) said transposase is configured to transpose said heterologous cargo nucleotide sequence to a target nucleic acid locus; and
(ii) said transposase comprises a sequence having at least 80% sequence identity to any one of SEQ ID NOs: 7, 8, 5, 6, 1-4, or 9-349.

158. The engineered transposase system of claim 157, wherein said transposase comprises a sequence having at least 90% sequence identity to SEQ ID NO: 7.

159. The engineered transposase system of claim 157, wherein said transposase comprises a sequence having at least 90% sequence identity to SEQ ID NO: 8.

160. The engineered transposase system of claim 157, wherein said transposase comprises a sequence having at least 90% sequence identity to SEQ ID NO: 5.

161. The engineered transposase system of claim 157, wherein said transposase comprises a sequence having at least 90% sequence identity to SEQ ID NO: 6.

162. The engineered transposase system of claim 157, wherein said transposase comprises one or more nuclear localization sequences (NLSs) proximal to an N-terminus or a C-terminus of said transposase.

163. The engineered transposase system of claim 162, wherein said one or more NLSs comprise a sequence having at least 80% sequence identity to a sequence from the group consisting of SEQ ID NOs: 455-470

164. The engineered transposase system of claim 157, wherein said double-stranded nucleic acid comprising a flanking sequence flanking said heterologous cargo nucleotide sequence, wherein said flanking sequence comprises a sequence having at least 70% sequence identity to at least 90 consecutive nucleotides of any one of SEQ ID NOs: 356, 357, 355, 354, 350-353, or 358-454.

165. The engineered transposase system of claim 157, wherein said transposase is configured to recognize an insertion motif adjacent to said target nucleic acid locus.

166. The engineered transposase system of claim 165, wherein said insertion motif comprises at least three consecutive nucleotides of the sequence AATGAC.

167. The engineered transposase system of claim 157, wherein said double-stranded nucleic acid comprising said heterologous cargo nucleotide sequence and said transposase are in a cell, and wherein said target nucleic acid locus is in said cell.

168. The engineered transposase system of claim 167, wherein said cell is a eukaryotic cell.

169. A method for modifying a target nucleic acid locus, comprising:
contacting said target nucleic acid locus with:
a transposase comprising a sequence having at least 80% sequence identity to any one of SEQ ID NOs: 7, 8, 5, 6, 1-4, or 9-349,
wherein said transposase is complexed with a double-stranded deoxyribonucleic acid polynucleotide comprising a heterologous cargo nucleotide sequence.

170. The method of claim 169, wherein said transposase comprises a sequence having at least 90% sequence identity to SEQ ID NO: 7.

171. The method of claim 169, wherein said transposase comprises a sequence having at least 90% sequence identity to SEQ ID NO: 8.

172. The method of claim 169, wherein said transposase comprises a sequence having at least 90% sequence identity to SEQ ID NO: 5.

173. The method of claim 169, wherein said transposase comprises a sequence having at least 90% sequence identity to SEQ ID NO: 6.

174. The method of claim 169, wherein said target nucleic acid locus is in a cell.

175. The method of claim 169, wherein said transposase inserts said heterologous cargo nucleotide sequence into said target nucleic acid locus.

176. An engineered transposase system, comprising:
(a) a double-stranded nucleic acid comprising a heterologous cargo nucleotide sequence, wherein said heterologous cargo nucleotide sequence is configured to interact with a transposase; and
(b) said transposase,
wherein said transposase is configured to transpose said heterologous cargo nucleotide sequence to a target nucleic acid locus, and
wherein said double-stranded nucleic acid comprises a flanking sequence flanking said heterologous cargo nucleotide sequence, wherein said flanking sequence has at least 70% sequence identity to at least 90 consecutive nucleotides of any one of SEQ ID NOs: 356, 357, 355, 354, 350-353, or 358-454.


SEQUENCE LISTING (454 sequences)

SEQ ID NO: 1 - protein, 130 aa, synthetic construct
MLEQTRSTLHHCVSSLNYHLVLATTYRRKCMTGSMLDRLEAICRSTAAKWECEVLEFNGEANHVHLLLHNFVTHLKTVTSRLLRKEFADHLKRYYWNTPVFWSRRYCILTVGGAPLSVLKQYSEQQERPE

SEQ ID NO: 2 - protein, 426 aa, synthetic construct
MPNRKVTYRLYPNAEQEARLQETRGLHQRLYNTALEERIRVYQETGKGLSFADQCKALTHWRKTIPAWAKLNAQSQQVTLKRLDFAFQQFFRRLKHGKRPGFPRFKSLRRYPGWGYKTHGDGWKLHPGAAGKHGKLYVQGIGMVPFRGKARTPGTPVTCDILHKVGKWYASVTLKIEAIQRERGTEMGAFDWSLMDFLTIATPKGLQTVTNPRHLKNQMAEVKRLGQAVSRKIRMAQRISGKQKGFPVSASLRKAIQQLARLHAKIARSRKDVLHQTSAWLVKRFGALATEALDVKNMVHHGGVRTKGLNREIHDAAPGAFLQMIKTKVEEAGSWYEEAPTREIRPTQRCHACWRVPEEKKTLSDRQHQCPHCGVTCDRDENAARVLLRWLETRLAGLRGSGREPSEVWSGRSFAAMKHETHAIAV

SEQ ID NO: 3 - protein, 138 aa, synthetic construct
MTQTTDIRHGRHCVFNLHVHLVFVTKYRRDVFTKTILDDLRGIFAAVCLDFGAELVEFDGEDDHVHLLVNYPPKVAISNLVNSLKGVSSRLIRKKHYPSICKKLWGGRLWSPSYFAGSCGGAPISIILQYIEQQQTSD

SEQ ID NO: 4 - protein, 412 aa, synthetic construct
MLQRKAVRYRLEPSSEQEHLLSRAVGCVRFVWNRALAIQKSYLEHGCGILSYVELAKCLTTWRNSEAFGFLSESPVHPQQQVLKFLERALKDAFDKKSPQRFPVFKKKGLHDSLRYPDPEQVKLDLHTADDARRRVFPKIFLPRIGWVKFRKSCIIGGEIRNVTVSRTGGHWYVAIQVEKEIPDPVHPSTTAVGGDRGVANLLTLSDGTMFLPVTAYRDLQGKLAREQRKLTKKVKFSANWKKQKAKITQLHQRIAEIRKTALHQVSQAISKNHAVVVLEDLKVQNMTASAKGTVEVPGRKVRQKAGLNKSILDQGWGQLRQMLGYKLQWAGGMLLLVDPAYTSQICPVCGVVDADNRKTQAVFRCQHCGHEDHADVNAAKNILARGLHPAKGLHSAQESTAGHAGVACFAA

SEQ ID NO: 5 - protein, 142 aa, synthetic construct
MKMADYHTNRHSCYLLEYHLVVVTKYRHPVISGKLKERLLEISYEVIENHWSCKISEINTDKDHIHIMFEAPPQVQLSKLANNYKTVTSRLLRKEFTDELKNYYWKPYFWADSYFVCTVSDKNRTVVKKYIQNQGVDKHKEH

SEQ ID NO: 6 - protein, 466 aa, synthetic construct
MATVSYGLKIASAYHIFDETIKIYRKAVKYITDVALFRFDEISVIDAYEGYTKQQAQRKYVESLIHATKESPACYERFDMEFYKFPSYLRREAITTAIGIVSSYKTNLQKWEANGKKGRRPHLNRNQNVMPCFYRNNTFLDSEETNIVSLKLYNGKDWIWETSVIRDCDFMYAYSHMKEWKASAPVLVKRNHRYELRTSYEMANSKFSKFKKDKEVETVIGVDLGINTDAVCSVVHKDGTVTGQRFINHPVEKDRMHGLLNTIKKAQQNGNHKTPRLWRLANNYNEAIAVKTAVEIVSFAMESKADIIVFEHLGMKKKKKGNKQKLSLWRKRDIQHRVEALAARNGIRVSYICAVNTSRLAFDGSGKVIRGKDAGFNTYELCKFTTGKVYNCDLSASKNIGARFFIRVLLKSLSAKEELLVLAKVPELNRRTSCCLATLINAYAVLCASKAKPEASAEGNAARQSH

SEQ ID NO: 7 - protein, 130 aa, synthetic construct
MEYKSNHNVVYSCKYHVVWCPKYRRKVLVNGVDIRLKKLIEEICGEYHIDIIEMEIMPDHIHLLMEVDPQFGIHKAVKLIKGRTSRVLRQEYGWLRSRIPSLWTNSYFVSTVGEAPLSVIKQYIENQKNV

SEQ ID NO: 8 - protein, 370 aa, synthetic construct
MEYSYKFRVYPTAAQARQIQRTFGCCRFVWNHYLALRKDFYEQDGKTMNYSTCSGDMTQLKKTLPWLREVDATALQSSLRDLDTAYQNFFRRVKQGQKPGYPKFKSKHHSKKSYKSKCVGTNIKVLDKAVQLPKLGFVKCRISKDVKGRILSATVSQNPSGEYFVALCCTDVEIVPLTSTGAVAGIDMGLKAFAITSDGVEYPNHKYLTRNQKKLAKLQRQLSRKSRGSNRREKARIQVARLHEHVSNQRQDMLHKLSTSLVRTYDLIAIEDLAPSNMVKNHKLARSISDASWGEFRRQLEYKAAWYGKQVVTVDRFFPSSQLCSTCGAQWPGTKDLSVREWACPKCGTIHDRDVNAAKNILNEGLRLLA

SEQ ID NO: 9 - protein, 136 aa, synthetic construct
MDLDKNRHSVYKLCYHLVVVTKYRHPVINKDINERLKQIAINIFEKAWNVRIVQMEIKCDHIHILFESSPQVQLSKLVNNFKTVSARLIRKEFKEQIDKYYCKSLFWSDSYCIISSGGEPIEIIKKYIENQNKPTI

SEQ ID NO: 10 - protein, 450 aa, synthetic construct
MDIIKTAKCKILNCASIFDDTVNIYRSALSFYIDVCYKEWDRFSTLESKYRNNYIEQITHRTSKNPSVKYDFDSSFYKFPCYLRRAAISEAIGYVSSFKSQLSNYKETDKKHKAPTLNLNPSSYPTLYDKGNWIKLSDGKARIKIYRNNDWVWLDINYDTKSLYSSKRYRFAGYEELSPTIRKYGKKYFLHIGFKNKVKLTDSKEIACSVDLGINTSATCSIMTSDGTVIARSFINQASEKDRLNSLLGKLSKATRNSGIKNSKPNLWRKINNIKKHIAIDTMNKIIAFAQANNATVIVFEYLSKMRVPKGFYGARRLRFKLQYWTKQLIYHKTLDKAHSLGLRVSRVSAKNTSHLAYDGSGMVSRGEIDTTKLYKNKPSKNYSICKFTTGKIYNADLSASYNIGARYFIREIIKPLSEKTRLQLEAKVPLLAARNQHTLSTLISLKAAC

SEQ ID NO: 11 - protein, 133 aa, synthetic construct
MIERNLHAVFDLEYHLVIVTKYRHPVLTEEVKASLLSHTYRLFETNFDCKVLEVNTDKDHIHILFSAKPQVQLSKLVNTYKTVTSRLLRKEFAEFLKPYYWKPYFWSRSYFICTASECSHDTVTVYIKNQGIN

SEQ ID NO: 12 - protein, 444 aa, synthetic construct
MNITSSYKMKLTGDLKALEISINIYRDALRILIPIINDNWETLSEYEFTKQKYNCIGNWIHNTKDNQARYNFDEQFPKFPSYLRRSAVAQALGIVSSYRSNLANWEKDPKGQAPQLSLTHYAYPAYYKRDLFRNFDPIRQTVELKVFKNGDWVFEVYKLKTSDCTYYHTHLTGKKQNVPIIQKKGRRFYAAFSYEEEVQLVPEESISKICAVDLGLNTDATCTIMGQDGTVYARTFISFSKEHDRLDTQLGRIKRNQKRGSHHNKKLWRSVAGISQDIADKTVQAILDFGNEHGVDVFVLEYLDFKGKNIVKRAHFWRYKRIYKVLAQRAHQYGLRIARVNARYTSRLAFDGSGWSKRGREIAPETPYALMEFPTGKTYNADLNASYNIGARYFIRHLLKTVTATQRLALEAKVPQVAKRSTCTLSDLINLRSELAVLTAGTQA

SEQ ID NO: 13 - protein, 137 aa, synthetic construct
MSYRTNRHSCYDLEYHLVVVTKYRHPVIDGAFKDRLTELTYRIFEENFQCYVNEVNTDKDHIHILFEAPPQVALSSLVCSYKTVTARLLRKEFAEYLAPFYWKPYLWSRSYFVCTVSERNHELITEYIRNQRNKEKG

SEQ ID NO: 14 - protein, 459 aa, synthetic construct
MAKIVSSYCTKVIGENRIFKDTIRVYRDALSLLIRIVESEWDTGLGELYAKSTLKAQRTVELLVHASRTSVAKYPEFDKQFYKYPSYLRRSTISEALGAVSSYRAAVSHWESAGRKGKEPKLQASRSAMPTFYKDNMYEDNGDGTARLKLYHKNDWVWVKVRLRKQDIDYIREHWDKADASAPKLVKRDRRYALCFAFEETVAYGTKPLTDQTILAVDLGINTDAVCSVMRPDGTVLARKFIDFPSEKDRLNTQLGRARRGQRENGHRGGNRHMKKAVRINEDLSKKIAPAIVRYAVSVGADIIVMEHLDFIGQKPKHKKHRLHMWRNRDIQKSVMHQAHRNGIRVRFVNARNTSRLAFDGSGEVARNSTNMALCRFRNGKQYNCDLNASYNIGARYFIREYLKPIPETEWSLIVAKVPELERRTNCTLSTLFSLYAVLNCQSVSVSESWPHGGNESGA

SEQ ID NO: 15 - protein, 137 aa, synthetic construct
MSYRTNRHSCYDLEYHLVVVTKYRHPVINGAFKDRLTELTYRIFEENFQCYVNEVNTDKDHIHILFEAPPQVALSSLVCSYKTVTARLLRKEFAEYLAPFYWKPYLWSRSYFVCTVSERNHELITEYIRNQRNKEKG

SEQ ID NO: 16 - protein, 108 aa, synthetic construct
MTGAMLDRLEAICRNTALKWECEVLEFNGEADHVHLLLALTPKVLPSAFVNNLKTVTSRLLRKEFSEHLKKYYWSKPVFWSRSYCILTVGGTPLSVLKQYIEQQERPE

SEQ ID NO: 17 - protein, 428 aa, synthetic construct
MLNRKVTYRLYPSASQAERLEEMLGLHRRLYNTALEERIRVYLETSKSLSFADQCKALTQWRKAVPALAGLNAQSEQVTLKRLHLAFQHFFRRIKNGETPGFPRFKSIHRTPGWGYKTHGDGWKLHPGENGAHGKLYLQSVGMIPMRGKPRQAGKPVTCEVLHKAGKWYASVTLEIQTIQRERGTEMGAFDWGVTDFLTIATGQGIETVKNPRHLRNQLSELRRLSREVSRKIVAAQKTSGRKKGFPVSRNLRKAIHALSRLHAKIARQRKDFLHQTSNWLVCRFGALATEALNVQSMTKSGKGSRKRGLNREVLSTSPSAFLQMTKTKAGEAGSWYEEASTREIKPTQRCHACWQLPEKRKTLADRQHQCPHCGNTCGRDENAALVLLRWLEKRLAGESGLCAPQGAGQELPEVWSGVRPRETVAPA

SEQ ID NO: 18 - protein, 138 aa, synthetic construct
MTTQNLSTLHHCVYNLTYHLVLVTKYRRRCMTGAMLDRLEAICRNTALKWECEVLEFNGEADHVHLLLALTPKVLPSAFVNNLKTVTSRLLRKEFSEHLKKYYWSKPVFWSRSYCILTVGGTPLSVLKQYIEQQERPE

SEQ ID NO: 19 - protein, 145 aa, synthetic construct
MILASIGRSVSDLKIHLVLTTKYRRKAINSEMLQRLHVILEDLLVKWDCKLVEFNGESDHVHVLFQYHPDLQLSTLVGNIKSTTSRRLRQEFTEHLSHFYTKNVFWNGSYFVASCGGVTISTLKHYIESQDRPENDNSSSTNIAD

SEQ ID NO: 20 - protein, 144 aa, synthetic construct
MQYAWLVDAAQGFRSSGNARFRLFAHLVLVTKYRRRVLTGPMLDRCREILARLCETWDCTLIECSGEPDHVHALLDLPPKVRPSDLINLLKTVSSRLLRSEYPALRADYRGKPVLWSPSYFLVSAGGAPIEIIRRYIEQQEKPG

SEQ ID NO: 21 - protein, 144 aa, synthetic construct
MQYAWLVDAAQGFRSSGNARFRLFAHLVLVTKCRRRVLTGPMLDRCREILARLCETWDCTLIECSGEPDHVHALLDLPPKVRPSDLINLLKTVSSRLLRSEYPALRADYRGKPVLWSPSYFLVSAGGAPIEIIRRYIEQQEKPG

SEQ ID NO: 22 - protein, 137 aa, synthetic construct
MSTNYDKGFRSVYLLTAHVVLVTKYRRQVINKALHNRLNEIFDDTCKKWKCTLLEFNGESDRVHLLIRYHPQVQINKFIANLKTVSSRLIRKEFPDYLYQFYKKPVFWTGSYFVASCGGVTIEQLKKYVEQQATPEN

SEQ ID NO: 23 - protein, 147 aa, synthetic construct
MKDDFVSKGRSISDLKVHLVLTTKYRRKALTDGMLKRLHIMLEDLLVKWDCKLIEFNGEEDHVHILFQYHPDLQLSTLVGNIKSTTSRRIRQEFAQHLSYFYSKDVLWNGSYFVASCGGVTITTLRQYIESQDRPAPGNSSATPLPG

SEQ ID NO: 24 - protein, 137 aa, synthetic construct
MEKDNEIRHGRHCVFKMHVHLVFVAKYRRKVFDGDAIDRMRRMFGKVCEDMGASLVEMDGEDNHVHLLVEYPPKLAVSVLVNSLKGVSSRLLRKERPDLQKRYWKNVLWSPSYFASSCGGAPISIIKQYIEQQQTPI

SEQ ID NO: 25 - protein, 146 aa, synthetic construct
MENQANKSSSHAVYSLKLLIVFVTKYRRKTLTPELLEYLREAFAEILEGWRCTLLEFGGEADHVHLLVEIHPALNISTLINNLKTASARRARNRFAEHLKPFYWKPYFWHRAYYVGNVGGATFETVRRYVESQGTKEKPRKVQQPA

SEQ ID NO: 26 - protein, 135 aa, synthetic construct
MSEYKVRYKSNNNVVFSCKYHVVWCPKYRRDVLVNGAVARLKEIIRQVCLETDSEVLEMEVMPDHVHLLVEVDPQFGVHHLVKLIKGRSSRLLRQEFRWCRTRLPSLWTNSYFVSTVGGAPLEIIKQYIEQQKHV

SEQ ID NO: 27 - protein, 135 aa, synthetic construct
MNNNWKSKNRHKYLLQYHLIFVCKYRKKLLSNKIISDDIKQFSYEICNKHNVIIHEMETDKDHIHYMIETEPNINLSNLVRTMKSYITYHIWKSHKKYLSKEFWRENTFWTDGYFICSIGNVSEKQLRKYIENQG

SEQ ID NO: 28 - protein, 121 aa, synthetic construct
MSLINYHFVFCPRYRRKVLVGQVEVRLKELLNQVCGEMEIEILAVECDVDHCHLFINAPPTLSPTDIMARVKGVTSRILRQEFEHLQHLPSLWTRSYFVSTAGNVSSETIRKYVEEQKTRG

SEQ ID NO: 29 - protein, 136 aa, synthetic construct
MDNNSLIHARTCVYNVNYHIVWSVKYGKPVLTEDIQEFLKDQFIKIGNEKGFSIAAMEVMPDHVHVFASAHPQIAPSYIVKMLKGISARRTLMQFPHLAQQLWKGHLWNSSFYIETIGSISEDAIKKYIEHQKTRG

SEQ ID NO: 30 - protein, 133 aa, synthetic construct
MNGNRWKRSATTVYNIGYHLIWCPKYRRKVLVGEVAERLKELLFQKAQEIGVEIVQMEVMPDHVHLFMKTAPTNSPHFIVHQLKGYTSRVLRKEFPFLKSRLPSLWTRSYYCESVGHISEETIRKYIEDQKGK

SEQ ID NO: 31 - protein, 117 aa, synthetic construct
MNYHFVWIPKYRRKILIDEIKDELEKLIHKIAKKHKIEVLSLSIQPDHIHLFVSAPPRLAPAQIANLFKGISSKKLLEKFPHLKTKEGLWSRTYYVGSAGSVSEETIRRYIEECQDM

SEQ ID NO: 32 - protein, 136 aa, synthetic construct
MNTYKSTRHAKFLINYHFVWIPKYRKDFLKDPEVKNTVEETIRELSKTYKFDILALEIMPDHIHLFISALPRYSPSELINVIKGATGRKIGQKFPQYKQRDSVWTRAYFVATAGNVSAETVKKYIESQWKKVEKDG

SEQ ID NO: 33 - protein, 115 aa, synthetic construct
MDIDKLHQWRGGRSCIYNNRYHLVFTPKYRKCVFDDRLLGALHEVFEQTSLQMKGFLHEFNGESNHVHLLVDIPPQVAVSAFVGKLKGKSSYILRRDHMDLISKIKTDPRNAIKS

SEQ ID NO: 34 - protein, 151 aa, synthetic construct
MILYWLIEVNPSTNIMRKDSFGYRRGNHSVGLATIHLVWIPKRRKPVLKGDIKLRLATILESVATDREWIIKAKEIASDHVHLLVEYDEQTPICEVVKAFKGRSSKLLRDEFPELKKLPSLWTRSYFYDTSGKVSTAKVIAYINDPHHERH

SEQ ID NO: 35 - protein, 127 aa, synthetic construct
MLPLPEYQTHNHVKFLLNYHFLFIPKRRKKVLVGGIATRLREIFLALAIEKNWDILALEVAPDHVHLFLAIKPIDTPALVIKAFKGRSSHHLRAEFPELKKLPSLWTSSPALLVPQAISAVRLLRST

SEQ ID NO: 36 - protein, 135 aa, synthetic construct
MGDNWKSKNRHKYLLQYHLIFVCKYRKKLFLNQNISDDIKQFSYEICQRHNVVIHEMETDKDHIHYMIETEPNINLSDLVRTMKSYTTYHIWKHHKNYLSKYFWKENTFFTDGYFISSIGNVSEKQLRKYIENQG

SEQ ID NO: 37 - protein, 141 aa, synthetic construct
MYKRKTGQTYYTNRHSCFLLQYHMVLVTKYRHPVLTGTIRDVVYEKVQSIFKDRGLEILEFNGEADHIHILFEADPLTAPGELVNVIKTQTSRTARKLYGDTLLKKWYWKPYFWSDSYFITTVSENSLQNVQAYIQSQGIK

SEQ ID NO: 38 - protein, 137 aa, synthetic construct
MKKLDNGYYVNRHSCFLLQYHLVFVTEYRKNVINKDIEEFLTTYFGKLMKNADCNLLEINYDKNHVHILFEAPVTLVLTNFINGLKTNSAKQLRSLYKEHIDNFYNKPVFWSRSYFIGTVSEQTTSAVREYIKNQGE

SEQ ID NO: 39 - protein, 147 aa, synthetic construct
MELDRKKWLHMLHKERTNVYDFHYHVVFVTKYRRQIFDTKTKQDNMKNILKKISQGNDSEIEHLEVMPDHIHLMLSFKPKYAPSSIVKSFKGASARAWFKQYPETKKQLCKGHLWSPSFFISTIGNVPEKVLAQYIESQMQKNFRQN

SEQ ID NO: 40 - protein, 132 aa, synthetic construct
MAQKYRHCNTSVSLLNYHFVWIVRRRRPILEGKLDERLKELIQQICPEIECEILALETDRDHVHLFVNSLPVLSPAQIIQRIKGSTSHELRKEFPWLKTKLPSLWTRSYFVSTAGNVSGDTIRRYIDSQRSR

SEQ ID NO: 41 - protein, 136 aa, synthetic construct
MGENLIHSRTCVYNINYHVVWSVKHRKKILTAEVEAYLKELVQQIADEKGFTVHLFEVGEGDHIHCFVSGPPKLSITDIVKYLKGITGRKLFEKFPEIRTRLWKGQLWNHSYYVETIGSVSEENIRRYIEHQSKSY

SEQ ID NO: 42 - protein, 143 aa, synthetic construct
MCMEVVGMNHYTHGRTCVYNINYHIVWCVKYRRKVLTPQIAERFYELINIIGEEKGFRIVECKVGEGDHVHCFVSAPPKISVTQIVKYLKGISGNKLLKEFPEIRKVLWNGQLWNGSYFCETVGSTSEETIRKYIERQKNCQL

SEQ ID NO: 43 - protein, 135 aa, synthetic construct
MKENDDLRHGRTCVYNLHAHLVFVTKYRRNVLTHDMLTDMETIMRDVCQDFDATLMEFNGEDDHVHLLVSYPPKVALSTLINSLKGVSSRRLRAKYDIKTHRNHLWSPSYYAASSGGAPLETLKQYIQQQRRPNQ

SEQ ID NO: 44 - protein, 142 aa, synthetic construct
MIYLKTTEYKRNRHSCYDLSYHLVVVTKYRHPVIQGALKKRLIELSHRILEENYPCEIHEINTDLDHIHIMFDAPPQVQLSTLVNNYKTVTARLLRKEFSDQLSKYYWKPVFWSQSYFIGTVSERSKSVVAQYIRDQGKERG

SEQ ID NO: 45 - protein, 137 aa, synthetic construct
MSRGIIPDAATRWARYRIGYHLVWIPKYRRPILTPEVGETVRTSLRAACADHDLTLLACETDRDHVHVFVSARPAVSPAHVARLLKGASSRAVRRAHQQAAARCGPRGLWAQSYYVGTVGDMSADTVRHYIENCQGR

SEQ ID NO: 46 - protein, 160 aa, synthetic construct
MDKEYEWRTGRSCIYDINLHLVFVTKYRRGVFTIQMLVRLNEIFKETCEQMDCKLHEFNGEDDHVHLLISLPPKLALSNLVGKLKGKSAYFIRKEYWDQIKNKLWGKHLWSPSYCAVSCGGAPLNVIKQYIQNQQKPACPKSIKKSIALTGQKRTRLTHP

SEQ ID NO: 47 - protein, 138 aa, synthetic construct
MNKSNDIRHGRHCVFNLHIHLVFVTKYRRSVFTKAVLEDLKEIFASVCQDFEAELVEFDGEKDHVHLLINYPPKVSVSRLVNSLKGVSSRLIRKKRYACIQDKLWAGALWSPSYFAASCGGAPISIIKQYIEQQDTPE

SEQ ID NO: 48 - protein, 139 aa, synthetic construct
MKIRGDGYYVNRHSCFLLQYHLVLVTKYRRPAITGEVEKGLKEYTVRYFGERGCVLHEMETMPDHIHLLFDAPPQVRLSDFVNAYKSASSRMVRKQYPEQLRAYYWKPYFWSLSYFIGSVSDRTAAAVAAYIRDQKNGK

SEQ ID NO: 49 - protein, 157 aa, synthetic construct
MEQNTIVLDFEITYNTHMNIAYKSNNNIVYSCKYYVVWCPKYRRKILTNGIDTRLKELLLEYAANISVDIMEMESMLDHVHILMEVAPQFGIHKAVKSLKRYTSKVLRSEYPSLKTRMPSLWTNSYFVSTVGEAPFDVIKQYIENQKTSQRQKDKLG

SEQ ID NO: 50 - protein, 146 aa, synthetic construct
MTENTHDWRTGRHVVYELHAHIVLVTKYRRKVMSPRVTKLLGDTFREVCERFECELEEFETDKDHAHLLVAYPPKVQLSKLVMSLKTNGSKRVREQDWPEVRQALWGDHFWSPSYCIVSCGGAPLEIIKRYIQDQQKPNRKYVRRS

SEQ ID NO: 51 - protein, 137 aa, synthetic construct
MSYRTNRHSCYDLEYHLVVVTKYRHPVIVGAFKERLLELTKRIFEENFSCFISEINTDRDHIHIMFEAPPQVTLSSLVCSYKTVTARLLRKEFAEYLKPYYWKPVLWSRSYFICTVSERSHTMMQEYIQNQGCKEKG

SEQ ID NO: 52 - protein, 130 aa, synthetic construct
MEYNRSSHYVYSCKYHIIFCPKYRSKVLKDDIEIRFKEIMKQLEIEDDFHIVEMEVMPDHIHLLLDCRPDKSPVEIVKHIKWQSAHILRKEFPELKTRLPNLWTRSAFIASVGSISLETVKEYIKNQKGK

SEQ ID NO: 53 - protein, 136 aa, synthetic construct
MRKVKNGSYYINGHSCFLLSFHLVLVTKYRKPVLSGDVKDTVYGVVKDVLHERGCALQAINGEANHVHVFFDAPPDVNLLDTVRVIKTKSARFSRSRYPAKVAQYYWKPYFWSDSYFIASVGDNTRDTVEQYIQNQ

SEQ ID NO: 54 - protein, 137 aa, synthetic construct
MTSKSDYRRGRSCVTAIHVHLVFVTKYRKDVFDASAIEALRGIFKEVCADFEAHLIEMDGEDDHVHLLVEYPPKVSVSSLVNSLKGVSSRLLRKSRPDLAHRYWSGVLWSPSYFAASCGGAPISVVRQYIEQQKTPD

SEQ ID NO: 55 - protein, 132 aa, synthetic construct
MMKNSHQVYELGYHIIFCTKYRHKVLDGDVAVACRNSIAETCAAYGWGLEEIEVMPDQVHMFVTASPQTAPAEIARTVKSISAVRIFTQFPKLKGRKFWGSGLWSPSTYFGSVGHISEETVRRYIQTQKERT

SEQ ID NO: 56 - protein, 134 aa, synthetic construct
MDNRYYRHNRRKYSLKVHIVLVTKYRKQLLQGSIADDVKQKILDIANTRGYEIIAMETDKDHIHFLLSYDATDRVCDIVKIVKQETTYYLWQKYNSVLSKQYWKKKIFWSDGYFACSIGEVSSATIQKYIESQG

SEQ ID NO: 57 - protein, 134 aa, synthetic construct
MKYSDDLRHGRTCIYNLQAHLVFVTKYRRGVFTKPMLEDMKQIMDKICRDFEGELIEFNGEDDHVHLLVSYPPKVPLSALINSLKGASSRILRRKYEIRTHRNHLWSPSYYAASSGGAPLETLKQYIRNQQTPD

SEQ ID NO: 58 - protein, 138 aa, synthetic construct
MQENNEIRKGRHCVFDMHVHLVFVTKYRHPVFGKQHLKHLERIFGDVCENFDCRLEEFNGETDHVHLLVSFPPTVELSRLVNSLKGVSSRYLRRDYPELAQHYWRAKRLWSGSYYAGTSGGAPLSTLKKYIQNQNRPQ

SEQ ID NO: 59 - protein, 134 aa, synthetic construct
MKYSDDLRHGRTCVYNLQAHLVFVTKYRRGVFTKPMLEDMKQIMDKICRDFEGELIEFNGEDDHVHLLVSYPPKVPLSALINSLKGASSRILRRKYEIRTHRNHLWSPSYYAASSGGAPLETLKQYIRNQQTPD

SEQ ID NO: 60 - protein, 144 aa, synthetic construct
MKIRRERHRVWSLSYHAVFVVKYRKPCITDEIADFLLDEMRHLLESWGGELIEGKADRDHLHLLFSLPPDKELARYIGLMKQVSARQVRKKYKDHLKEYLWGDSFWTDSYYLGSTGGANLEVIEEYIKKQGQPKRKYVRKNGLS

SEQ ID NO: 61 - protein, 152 aa, synthetic construct
MADMGKMDKVKHNNNVTYRCHYHVVWCPKYRRKVITSLDPKLDNPPIDGDPGPVDERLKQIIREVCKETGNDILELETMPDHVHLLVDCDPQYGINKLVRLIKGRTSRYLRSEFPSLKRRLPTLWTNSYFVATVGGAPLSIVKQYVQNQRNV

SEQ ID NO: 62 - protein, 164 aa, synthetic construct
MNCPSFVYVVHMDILKCDIIIDMKDTERLTYGRTSVYNLNYHIIWGTKYRNKVLKGHVEVAFKRIILEIADKYGFEIAHLEIGKDDHVHLLVSAPPKLSVTNMVRWLKGITAYRLFRECPELKNSYWKQRDRHLWSSSYYVESIGSVNKQAVAKYIDDQRKKEI

SEQ ID NO: 63 - protein, 147 aa, synthetic construct
MSASDERFTHGRTSVYNLNYHIIWCTKYRRKVLKNGIDSDLKTILREIADEHGYRIPRMEVGLDDHIHLFVSAPPKISVSSIVKQLKGTSSLRLFAMHPELKSQYWKRKGERSLWSPSYFAESIGSVNEQAVAKYIDDQRSKERESQ

SEQ ID NO: 64 - protein, 152 aa, synthetic construct
MADMGKTDKVKHNNNVTYRCHYHVVWCPKYRRKVITSLDPKLDNPPIDGDPGPVDERLKQIIREVCKETGNDILELETMPDHVHLLVDCDPQYGINRLVRLIKGRTSRYLRSEFPSLKRRLPTLWTNSYFVATVGGAPLSIVKQYVQNQRNV

SEQ ID NO: 65 - protein, 146 aa, synthetic construct; identical to SEQ ID NO: 50

SEQ ID NO: 66 - protein, 134 aa, synthetic construct; identical to SEQ ID NO: 59

SEQ ID NO: 67 - protein, 134 aa, synthetic construct
MELQRNSHQVYEIGYHIIFCTKYRHKVLVGEVETTCRNSIAETCLAYGWQLKELEIMPDHVHMFVSAPPQTAPAEIARTVKSISAVKIFTAHPKLKGRKFWGSGLWSPSTYFGTVGHISEDTVRRYIQTQKERG

SEQ ID NO: 68 - protein, 140 aa, synthetic construct
MNVKYKSNNNIVYSCKYHVVWCPKYRRKVLTNGVDVRLKELLLEYAANLSVDILEMEIMPDHVHILMEVAPQFGIHKAVKSLKGYTSKVLRSEYPFLKTRIPTLWTNSYFVSTVGGAPLEVIKQYIENQKTSQRQKDKLG

SEQ ID NO: 69 - protein, 138 aa, synthetic construct
MKENNPVRQGRHAVWDCHAHLVFVTKYRRGAFTEPILEDCENVMRKVCDDYGCTLDEFNGEEDHVHLLVTYPATVQLTRLVNSLKGVSSRILMRDHGQHLKHYLWGGHLWSRSYYCGTTGGANLETIKRYVQNQRRPN

SEQ ID NO: 70 - protein, 134 aa, synthetic construct; identical to SEQ ID NO: 59

SEQ ID NO: 71 - protein, 132 aa, synthetic construct; identical to SEQ ID NO: 55

SEQ ID NO: 72 - protein, 137 aa, synthetic construct
MASLSKKDLEYRHESNAVSLINMHFVFIPKRRKAVLVGKVAERLQEIIFEVVVENRWKIIALEIMPDHVHLLLNVKPVDSASFVMQRVKGRASSYLRKEFPELLKLPTLWTPSYFVGSVGNVSTETVRKYIEEQRNK

SEQ ID NO: 73 - protein, 136 aa, synthetic construct
MSNYSTYGYKTNRNVVYKCYYHVVWCPKYRRKVLHSGVDERLKQVVHEVCNELGAELMGLEVMPDHVHLLVGVDPQFGVHRLVKRIKGKSSRVLREEFPWLKSRIPSLWTNSYFVTTTGGAPLAAVKKYIENQKTV

SEQ ID NO: 74 - protein, 136 aa, synthetic construct
MSKKSQYISTNRSKHYLKYHLILVTKYRRNILVDQLNEDLKSIFSSIADGSDFEIEVFESDVNHIHFLIRYIPRLSVSQLVRRLKQESTRQLWLLHHSTLRQYYWYRKILWSDGFFVCSIGEASPETIRQYILSQG

SEQ ID NO: 75 - protein, 145 aa, synthetic construct
MHICYTVCMRKDNLVHGRTTVYNINYHIVWSVKYRRKIITPEVESYMKEVIQDIAESKGFKVDEFASGNEDHVHLFVTAPPKLSPSLIIQYLKGITGRKLYEKYPDLRSKLWKGELWNHSTYIETIGDVSEDTIRKYIEHQSRQY

SEQ ID NO: 76 - protein, 146 aa, synthetic construct
MSTRLRHHAHSSFRIWMHIVLVTKYRKKVITLEILDRLGEIFRKLCTTQKCELVEFNGEADHVHLLVDMAPDISVSKLVNILKTISSREVRREFPEHVNKFYWKSVFWTSAYCAVSSGGAPLDVLKRYIQDQNEPEPQCDKDPVRR

SEQ ID NO: 77 - protein, 138 aa, synthetic construct
MSTNYNKGFRSVYKLTAHVVLVTKYRKKAISEKVLLRLKEIFTDTLIKWECNLVEFNGESDHVHLLIDYKPDISLSKLIANLKTVSSRLIRRDFPELASKYFYSKPYFWTGAYFVASCGGVTVSQLKKYVENQKTPQK

SEQ ID NO: 78 - protein, 136 aa, synthetic construct
MKENLIYSRTCVYNINYHVVWSVKYRRKILSAEIEAYLRKLVQEIADDKGFTVHLFEAGEGDHIHCFVSAPPKMSVTDLVKYLKGITGRKLFEKYPEIRQKLWKGQLWNHSYYVETIGSVSEENIRRYIEHQSKSY

SEQ ID NO: 79 - protein, 137 aa, synthetic construct
MLEIVFDRNCVYQTAYHAVWCPKYRKSILVGSIAAETKRFIETICQERQWSVLSLEVQPDHIHLFVSIPPATSVADAIKILKGSTARKLFVKFPELKKQLWGGHLWSPSYYVGTAGNVSSETIQKYIERTEHIKGRR

SEQ ID NO: 80 - protein, 141 aa, synthetic construct
MQKKERVPDAIYERNYVYNFHFHLIWVTKYRNPAFTTPELVRDMKSILSKLAADKEISIEEMEVMPNHIHMLISFKPKYAPSDIVKYLKGHSAKQFFDKHPEIRGHEFWGGHLWSHSYYMSTLGNMSKEVVEQYIRNQYTK

SEQ ID NO: 81 - protein, 102 aa, synthetic construct
MDNRYNRHNRRKYSLKVHIVLVTKYRKQLLKGTIADDVKQKIFDICNSNGWNIIAMETDKDHVYFLVSYDATDRVCDIVKAVKQQIIYYLWQKYSDFLSEQY

SEQ ID NO: 82 - protein, 154 aa, synthetic construct
MVSRKIFVFLRKILYFCTMKKRKDIYDKLNHSKYHIWYHFIFSTKYRRKCLCGIEDEVRECFFDISKKCNFRVEHIGIDKDHIHLLIKSCPSMSPLSIVRRLKQLSTRILWNKCEVHLSKYYWKKHIVWTNGYFCSTIGEISEKNLIEYINNQG

SEQ ID NO: 83 - protein, 140 aa, synthetic construct
MRKKENTDYYVHRHSCFLLQYPIVLVTKYRHPVLTGDVKDTVYERIHYIAEQRGFRILEINGEPDHIHVLMEVDAITSPAELANVLKTQTARRARTLYGETLLKKYYWKPYFWSDSYFITTVSENSLKVVKQYIQNQGNR

SEQ ID NO: 84 - protein, 153 aa, synthetic construct
MEYRRRSHSVYLLTYHIVFVTKYRKPVITGEIGDFMKNHAAYLCQRFQGELIAAETDRDHIHLLVSMPPQVCPADLVRTLKTQLSKEVHLNMTYNAQVQEYLKGDVPLWSPSYFIATTGSSVMEKVKEYINAQRTDGHKRKYEKSGKYKKKDA

SEQ ID NO: 85 - protein, 138 aa, synthetic construct
MNKSNDIRHGRHCVFNLHIHLVFLTKYRRSVFTKAVLEDLKEIFASVCQDFEAELVEFDGEKDHVHLLINYPPKVSVSRLVNSLKGVSSRLIRKKRYACIQDKLWAGALWSPSYFAASCGGAPISIIKQYIEQQDTPE

SEQ ID NO: 86 - protein, 157 aa, synthetic construct
MDKIVIVLTNKPWYNTNMNTTYKSNNNVVYSCKYHVVWCPKYRRKVLVNGVDVRLKELLTEYAANLSVDILEMEIMPDHVHMLLEVDPQFGIHKAVKSFKGYTSRILRQEFPYLKTKMPTLWTNSYFVSTVGGAPLETVKQYIENQKTSQRQKDKMG

SEQ ID NO: 87 - protein, 138 aa, synthetic construct
MYKKKLGQTYYTNRHSCFLLQYHIVLVTKYRRPVLTGAVKELVYQVIQDIFDEKDLVILEMNGESDHIHILFEADPFTAPGTLVNVIKTKTSRYARTQYGDTILKESCQDSLFWSNSYFTTIANENSLQLIQEYINNQ

SEQ ID NO: 88 - protein, 138 aa, synthetic construct
MNKSNDIRHGRHCVFNLHIHLVFLTKYRRSVFTKAVLEDLKEIFASVCQDFEAELVEFDGEKDYVHLLINYPPKVSVSRLVNSLKGVSSRLIRKKRYACIQDKLWAGALWSPSYFAASCGGAPISIIKQYIEQQDTPE

SEQ ID NO: 89 - protein, 139 aa, synthetic construct
MQMFVNNNLSYGRGYVYNIQYHIVWCTMYRKPVLINETEQSLKESLQQTAETIKINIVEMECMPDHIHLLVECSPQHFIPDMIKVLKGNSARAIFIANPDLKKQLWGGHLWNPSYFIATVSDNTRKQVQEYIASQKIRN

SEQ ID NO: 90 - protein, 140 aa, synthetic construct
MRKKENTDYYVHRHSCFLLQYHIVLVTKFRHPVLVDGVRDAVYERIRYVTDQRGFHILEINGEPDHVHILLEADTMTSPAELANVLKTQTARRARKLYGDTILKKYYWKPYFWSDSYFISSVSENSLKVIQQYIKNQEIK

SEQ ID NO: 91 - protein, 135 aa, synthetic construct
MDYNRSGHYMYSCKYHIIFCPKYRKKVLVNGVDEDLKKIILDLQKDMYKVIEMEIMPDHVHLLLDCNPYKSPVDIVKHIKHQSALILKKKYPVELDQKMPCFWTRSAFISSVGTISMETAQAYIQSQKKNSRKEK

SEQ ID NO: 92 - protein, 132 aa, synthetic construct
MQEIYRSRYSAYLLHVHIVFVTKYRRKVFGELHLEKVNQYAGEVCGDFGAELNECDGKADHVHMLVEYPPTVQLSKLVNSLKAVTSRRLRNEFLDLREAYNKPVLWSRSYFVGSCGGAPLEVVKRYIQHQHG

SEQ ID NO: 93 - protein, 142 aa, synthetic construct
MDKTTYFSIPKDVTHGRGYVYSLQYHIVWVTKYRKPVFLGDIEEDVRNYLLETLKSLDMDVIAMEVMPDHIHLLVDCKPQLRLSDAIKILKGNTARWLFLKHPEIKTQLWGGHLWNPSYFVATVSDRSFNQVKSYIDHQKIQ

SEQ ID NO: 94 - protein, 131 aa, synthetic construct
MKLDSNNHSVFLLYYHLVLVVKYRRKVFSDQMSQYAKDIFVRIGSSYNITLEEWNHDQDHVHIMFRAYPNTELSKFINAYKSASSRLIKKDFPEVRRKLWKEMFWSRSYCLLTTGGASIETIRKYIEHQGR

SEQ ID NO: 95 - protein, 140 aa, synthetic construct
MYEKNPDGYYKNRHSCFLLSYHLVLVTKYRKPVLTGVVLQTVKDVILSVMKERGCIVHEINGEADHLHVMFDAGPEVNLLDLVRVVKTKSSRFCRSRHKNEVEKYYWKPYFWSDSYFITSIGESTRLLVQKYIQNQGSDH

SEQ ID NO: 96 - protein, 162 aa, synthetic construct
MKGIYAMFKKVKDKPYYTNNHSCFLLTAHLVLVTKYRNPVLQGNVKDYVYQLIRETMEAENIVIREMNGETDYVHVLFDYAPDIKLTALVNKIKSRTSRLVRRDYPLEVGKYYSKPMFWTNSYFLGSVGVNMAKIVEEYIKKNSNTEFTTALQGRRTLLNCL

SEQ ID NO: 97 - protein, 130 aa, synthetic construct
MRYKSNRNVVYSCKYHVVWCPKYRRPVLVNGVDGRLKDIIRETCAEIHAELIEMEVMPDHVHLLVEVDPQYGIHRAVRLIKGRSSRLLRDEYPWLKSRLPTLWTNSYFVSTVGGAPLKTIRQYIENQKHV

SEQ ID NO: 98 - protein, 151 aa, synthetic construct
MEYKKKAHSVYLMTYHLVFVTKYRKPVISNEIGEFMKELSREICEQHGGTLISAETDADHMHLLISMPPQERPSDIIRVLKTQTSKKVHLNEKFDKYIKQYLFGDVSLWSPSYFIATTGGVTIEKINEYVESQKTEYHKRKYEKTGRYKKK

SEQ ID NO: 99 - protein, 142 aa, synthetic construct; identical to SEQ ID NO: 5

SEQ ID NO: 100 - protein, 142 aa, synthetic construct
MDKTTYFSEPKDVTYGRGYVYSLQYHIVWTTKYRKPIFVGRIEKEIKNYLLNILKSLDMIPIAMEIMPDHIHLLVDCKPQLRLSDAIKILKGNTARWLFLSHPELKKQLWGGHLWNPSYFVATVSDRSLEQVKKYINEQKIK

SEQ ID NO: 101 - protein, 139 aa, synthetic construct
MQMFVNNNLSYGRGYVYNIQYHIVWCTKYRKPVLVNETEQSLKESLQQTAETIKINIAETECMPDHIHLLVECSPQHFIPDMIKVLKGNSARAIFIANPDLKKQLWGGHLWNPSYFVATVSDNTRKQVQEYIASQKTRN

SEQ ID NO: 102 - protein, 131 aa, synthetic construct
MKLDNNNHSVFLLYYHLVLVVKYRRKVFSDQMSQYAKDIFVRIGSSYNITLEEWNHDQDHVHIMFRAHPNTEMSKFINAYKSASSRLIKKDFPEVRRKLWKEMFWSRSYCLLTTGGAPIETIRKYIENQGR

SEQ ID NO: 103 - protein, 170 aa, synthetic construct
MRTNKNGVNKDQKDKVKKDGYNRGQHCVYCLTYHMIFVTHYRKPVINDEMSAAMKEFSNHMAEQFRGKVLSAETDRDHIHLLVSLPPNTNISVFVRSIKTQLSREMRKRFPEQIKQYIYGDDTSFWSRSYFIATTGSVSLETVKQYIESQRSEEHQAKKNQQFQKKTLLE

SEQ ID NO: 104 - protein, 132 aa, synthetic construct; identical to SEQ ID NO: 55

SEQ ID NO: 105 - protein, 133 aa, synthetic construct
MGNNYRHTNTTVSLINYHFVFCPRYRRKIFLISNVERRFKELVKIKCKELEIEIIAIECDKDHTHMFLNCLPTLSPSDIMQQIKGYTSKILREEFAELSKMPSLWTRSYFVSTAGNVCSETIKKYVENQKKRY

SEQ ID NO: 106 - protein, 130 aa, synthetic construct
MEYKSNNNVVYSCKYHVVFCPKYRRSVLINGVDVRLKELIQQTCEQLNVEIIEMEIMPDHVHLLMEVDPQFGIHKAVKQIKGYSSHVLREEFPWLKSRLPSLWTNSYFVSTVGGAPLAVIKQYIENQKNI

SEQ ID NO: 107 - protein, 137 aa, synthetic construct
MKNEYTYDRTCVYQNRYHIVWCVMYRRKVLRPEISNRLYELVQTIAKEKGFDIIECETYDCDHVSCIVSAPPKLSVTQIVKYIKGISGKIILKEYPEIVPLLWNNQMWNNSYFCETLGTVSDEAVNKYIERQKKCQH

SEQ ID NO: 108 - protein, 141 aa, synthetic construct
MQKKERIDGAVYERNYIYNFHYHLIWVTKYRNPAFTTPELVNEMKGILLQLAEIKEISIEEIEVMPDHVHLLISFKPKHAPADIVKYLKGHSAKMFLAAHPEIRNHDFWGGHLWSHSYYMSTLGNMSKEVVEQYIRNQYTK

SEQ ID NO: 109 - protein, 138 aa, synthetic construct
MDKTNDIRKGRHCVYDLHAHLVFVTKYRHDVFTDEHLKALERIFSDVCASFDCRLEEFNGETDHVHLLVSFPPTVELSRLVNSLKGVSSRYMRRDYPELAQHYWRAQRLWSPSYYAGTAGGAPLNTLKRYIENQNRPA

SEQ ID NO: 110 - protein, 138 aa, synthetic construct
MYKKKLGQTYYTNRHSCFLLQYHIVLVTKYRRPVLTGAVKELVYQVIQDIFDEKDLVILEINGEADYIHILFEADPFTAPGTLVNVIKTKTSRYARTQYGDTILKESCQDSLFWSNSYFTTIANENSLQLIQEYINNQ

SEQ ID NO: 111 - protein, 162 aa, synthetic construct
MKGIYAMFKKVKDKPYYTNNHSCFLLTAHLVLVTKYRNPVLQGNVKDYVYQLIRETMEAENIVIREMNGETDYVHVLFDYAPDIKLTALVNKIKSRTSRLARRDYPLEVGKYYSKPMFWTNSYFLGSVGVNTAEIVEEYIKKNSNAEFTTALQGRRTLLNCL

SEQ ID NO: 112 - protein, 149 aa, synthetic construct
MVEYVYRIYYTMCMKENLIHYRTSVCNINYHVVWSVKYRRKILNAEVEAFLKQLVVQIGNDKGFTVQLFECGNLDHVHCFITAPPKLSVTTIVKYLKGITGRKLFEQFPDIRFKLWKGELWNHSYYCETIGSVSEENIRRYIEYQSKSY

SEQ ID NO: 113 - protein, 136 aa, synthetic construct
MDKYTHARTCVYNINYHIVWCVKYRRKVLTPDISNRLYELVQSVAEEKDFKVVECKVVEGDHVHCFVSAPPKISVTQIVKYLKGITGNKLLKEFPEIRKSLWKGQLWNGSYFCETIGSTSEENILKYIERQKNCQL

SEQ ID NO: 114 - protein, 142 aa, synthetic construct
MDKNIVSHILSNVTHGRGYVYSLQYHIVFVTKYRKPVFTGEIEDAARNHLVDTCKQLSIDILAMELMPDHVHMLVSCKPQTCLSDAIKVLKGNTARWLFLSHPELKASLWGGHLWNPSYFVATVSERSFEKVEEYIKSQKTK

SEQ ID NO: 115 - protein, 133 aa, synthetic construct
MKKEYRRTKTTVFMINYHFVFCPRYRRKLFLIDGFEARFKELVTQICEQNDMVILAMECHIDHCHLFVNAPPTLSAADIMKIIKGNTSRQLKQEFFSNVDMQMWTRSYFVSTAGDVSNATIQRYVEEQKTRGG

SEQ ID NO: 116 - protein, 142 aa, synthetic construct
MKIVEYYTNRHSCYLLEYHLVVVTKYRHPVISGKLKERLLEISYEVMENHWNCKISEINTDKDHIHIMFEASPQVQLSKLANNYKTVTSRLLRKEFADELKNYYWKPYFWADSYFVCTMSDKNRNVVKKYIQNQGVDKHKGH

SEQ ID NO: 117 - protein, 139 aa, synthetic construct
MYEKNSAGYYKNRHSCFLLSYHLVLVTKYRNPVLTGEVKELVYDTIRAVLEERGCILTEINGEPDHIHVLFDAGPEVNLLDLVRVIKTKTSRFARSRCKEQVAKYYWEPVFWSDSYFITTVGENTRQVVQEYIKHQGGR

SEQ ID NO: 118 - protein, 142 aa, synthetic construct
MDKTAYFSIPKDVTHGRGYVYSLQYHIVWVTKYRKPIFTGSIEADVRQYLLETMQSLEMRPLAMEVMPDHIHLLVDCKPQLRLSDAVKILKGNTARWLFLKHPELKKQLWGGHLWNPSYFAATVSERTLEQVTQYINSQKTH

SEQ ID NO: 119 - protein, 130 aa, synthetic construct
MEYKSNNNVVYSCKYHVVFCPKYRRSVLINGVDVRLKELIQQTCEQLNVEIIEMEIMPDHVHLLVEVDPQFGIHKAVKQIKGCSSRILREEFPWLKSRLPSLWTNSYFVSTVGGAPLAVIKQYIENQKYV

SEQ ID NO: 120 - protein, 169 aa, synthetic construct
MNASTFYIYAGGIFMKDYTKTRHSVYLLTYHMIFVTKWRKPVITDEIGDFMIKTARRLCIGYGGELVSGETDRDHIHLLVSLPPSKNLIDIIRSLKTQLSKEVHAHPEYDRIVKKHIYGNAPLWSPSYFVATTGSVSMDVVKQYIEGQRTDEHKRKYEKRSLDYWNSRL

SEQ ID NO: 121 - protein, 170 aa, synthetic construct
MRANKNGVNKDQKDKVKKDGYNRGQHCVYCLTYHMIFVTHYRKPVINDEMSAAMKEFSNHMAEQFRGKVLSAETDRDHIHLLVSLPPNTNISVFVRSIKTQLSREMRKRFPEQIKQYIYGDDTSFWSRSYFIATTGSVSLETVKQYIESQRSEEHQAKKNQQFQKKTLLE

SEQ ID NO: 122 - protein, 157 aa, synthetic construct
MDKITIVLIDKTLYNTNMNTTYKSNNNVVYSCKYHVVWCPKYRRKVLTNGVDVRLKELLTEYAANLSVDILEMEIMPDHVHMLLEVDPQFGIHKAVKSFKGYTSRILRQEFPYLKTKMPTLWTNSYFVSTVGGAPLEAVKQYIENQKTSQRQKDKMG

SEQ ID NO: 123 - protein, 142 aa, synthetic construct; identical to SEQ ID NO: 93

SEQ ID NO: 124 - protein, 132 aa, synthetic construct
MKNEYNTSEHLVYLCRYHVIFCPKYRRKIFVDGIDVRTKEIFLETAKSHQFEIMEMEVMPDHVHLLISCNPRYGIMQCVTDLKRDSSYKLIREFPSIRKRIPCLWTRSAFIATVGSVSLETVKQYIESQKGK

SEQ ID NO: 125 - protein, 138 aa, synthetic construct
MSDTEYQKNRHSCYYLKYHLVVAIKYRHPVLGIEEIRTRLLELSYRLIEEDWGCHIDNINTDLDHVHILFSATPQIQLSKLVNNYKTVTARLIRKEFPEELKPFFWKPYFWSDSYFIGTVSDVTDKMISKYIDNQGKK

SEQ ID NO: 126 - protein, 132 aa, synthetic construct
MKNEYNTSEHLVYLCRYHVIFCPKYRRKIFVDGIDVRTKEIFLETAKSHQFEIMEMEVMPDHVHLLISCNPRYGIMQCVTDLKRDSSYKLIREFPSIRKRIPCLWTRSAFIATVGSVSLETVKQYIEFQKGK

SEQ ID NO: 127 - protein, 135 aa, synthetic construct
MDRVYHSNHNIVYSCKYHIVFCPKYRRKVLVNGVDVRLKELIHSIADENNFDVIEMEIMPDHVHLLLEVDPQFGIHKAVKTIKGKTSRILRQEFHWLTTKLPTLWTNSYFCSTVGGAPLEIVRQYIESQKTSQRK

SEQ ID NO: 128 - protein, 139 aa, synthetic construct
MYEKNNAGYYKNRHSCFLLSYHLVLVTKYRNPVLTGEVKELVSDTIRAVLEERGCILTEINGESDHIHVLFDAGPEVNLLDLVRVIKTKTSRFARSRCKEQVAKYYWKPVFWSDSYFITTVGENTKQVVQEYIKYQGGR

SEQ ID NO: 129 - protein, 134 aa, synthetic construct
MDNRYNRHNRRKYSLKVHIVLVTKYRKKLLKGSIADDVKQKIFDIANTRGYEIIAMEADKDHMHFLISYDTTDRVCDIVKIVKQETPYYLWQKYGSFLSKQYWKKRIFWSDGYFACSIGEVSSATIQKYIESQG

SEQ ID NO: 130 - protein, 151 aa, synthetic construct
MEYKKKAHSVYLMAYHLVFVTKYRKPVISNEIGEFMKELSREICEQHGGTLISAETDADHLHLLISMPPQERPSDIIRVLKTQTSKKVHLNEEFDKYVKQYLFGDVSLWSPSYFIATTGGVTIEKIKEYVESQKTEYHKRKYEKTGRYKKK

SEQ ID NO: 131 - protein, 138 aa, synthetic construct
MYKKRSGQTYYTNRHSCFLLQYHIVLVTKYRHPVLTGLVKDLIYQVIQDIFDEKKLVILEMNGEADHIHILFEADPFTAPGILVNIIKTKTSRYARKEYGETLLKEYYWKPLFWSDSYFVTTVSENSLQMVQEYIRNQ

SEQ ID NO: 132 - protein, 134 aa, synthetic construct
MPSDVTHGRGYVYSLQYHIVWVTKYRKPVFTGKIESDVREYLNETLKTLDMDVIAMEVMPDHIHLLVNCKPQLRLSDAIKILKGNTARWLFLSHPEIKNQLWGGHLWNPSYFVATVSDRSFKQVTEYINSQKTK

SEQ ID NO: 133 - protein, 141 aa, synthetic construct
MQKKERIPGAIHERNYIYNFHFHLIWVTKYRNPAFTTQELVDDMKAVLRQLAKEKGITIEEMEVMPDHVHLLISFKPKYAPADIVKYLKGHSAKQFFMMHPEIRNQEFWGGHLWSHSYFMSTLGDMSREVVEKYIRNQYSK

SEQ ID NO: 134 - protein, 142 aa, synthetic construct; identical to SEQ ID NO: 100

SEQ ID NO: 135 - protein, 134 aa, synthetic construct
MPSDVTHGRGYVYSLQYHIVWVTKYRKPVFIGKIESDIKEYLNKTLKSLDMDVIAMEIMPDHIHLLVNCKPQLRLSDVIKILKGNTARWLFLNYPEIKDQLWGGHLWNPSYFVATVSDRSLEQVTEYINSQKTK

SEQ ID NO: 136 - protein, 136 aa, synthetic construct
MSNTYKSGSHSKYLLQYHLIFVCKYRRKLLSSPNIASDIKRLSLEYAQKHNVVIHYMETDQDHIHYMIETTPNINLANMVKTIKSYITYHIWEKYSNYLSKCFWKERTFFSDGYFISSIGNVSQETLKNYIENQGK

SEQ ID NO: 137 - protein, 152 aa, synthetic construct
MADMGKMDKVKHNNNVTYRCHYHVVWCPKYRRKVITSLDPKLDNPPIDGDPGPVDERLEQIIREVCKETGNDILELETMPDHVHLLVDCDPQYGINRLVRLIKGRTSRYLRSEFPSLKRRLPTLWTNSYFVATVGGAPLSIVKQYVQNQRNV

SEQ ID NO: 138 - protein, 147 aa, synthetic construct
MSADDERFTHGRTSVYNLNYHIIWCTKYRRKVLKNGIDSDLKTILREIADEHGYRIPHMEVGLDDHIHLFVSAPPKISVSSIVKQLKGTSSLRLFAMHPELKSQYWKRKGERSLWSPSYFVESIGSVNERAVAKYIDDQRSKERESQ

SEQ ID NO: 139 - protein, 133 aa, synthetic construct; identical to SEQ ID NO: 115

SEQ ID NO: 140 - protein, 118 aa, synthetic construct
MVWTVKYRRKILTSEIESYLKELVQQIAKEKGFTVQLFECGNQDHVHCFITAPPKLSVTTIVKYLKGITGRKLFEQYPNIRNQLWKGQLWNHSYYCETIGSVSEENIKRYIERQSKSY

SEQ ID NO: 141 - protein, 151 aa, synthetic construct; identical to SEQ ID NO: 98

SEQ ID NO: 142 - protein, 131 aa, synthetic construct
MKYKSNNNIVYSCKYHVVFCPKYRRKVLNNGVDERLKELINNICQELHVDLIEMEVMPDHVHLLLEVDPQFGIHKVVKRIKGISSRILREEFSWLKSRLPTLWTNSYFVSTVGKAPISIIKQYIESQKRSE

SEQ ID NO: 143 - protein, 139 aa, synthetic construct; identical to SEQ ID NO: 89

SEQ ID NO: 144 - protein, 160 aa, synthetic construct
MNTLFSRTLSKYYRMRLAHGFALWYNRYMKKLNKSRHARYLLNLHIVWIPKYRKAVLGPYREALTDILHSIAKDKGWEILAVEVMPDHIHLFVSVLPSISPSEVVKCFKGQSARKLLVEFPELVQQRDYRASFWAPSYFVASAGDVSSETIKAYIEGQYD

SEQ ID NO: 145 - protein, 119 aa, synthetic construct
MVHLIFVTKYRKKIVLGDFRDAVKQYLFDTCVKNHWYVKRMETDQDHVYILLQYNPTDSITHIVSILKQHSTYLSWQQHSALLKQHYWKKHVLWSDSYFAASIGTVSQAVIKRYIENQG

SEQ ID NO: 146 - protein, 137 aa, synthetic construct
MSEEQYHSASHCKYLIQYHIIWCLKFRFSVLQGNVDIILKQILQKICNDYKYKIKALEVMPDHIHIFIDVPQTVAPCDVVRTLKSISAIELFKVFPKLKQFYAKCGVLWPRGYFVSTVGHISEATVIKYIEEQKNDK

SEQ ID NO: 147 - protein, 136 aa, synthetic construct
MELNSNRHSVYRLQFHLVVVTKYRLKVINQQMNDRLKELAHRVFEECWGLKILEIETESDHIHILFESNPQTDLSKLVNNFKTVSSRMLRKEFSEYLGKFYYKPYLWANSYCILSVGEASMEVIKHYIENQEAPQN

SEQ ID NO: 148 - protein, 139 aa, synthetic construct
MIKKTVENIDESRATNHSVFNILVEVTIFTKDKAALINENVNEIIYGLFTRISKEHGVQVVKWHHEDCSVQMLLSISPSTNLTKYINATKASSSRLLKKEIYGLSLALPDGKFWGRGFYAFSLDAKNKKTKNKKRVNIK

SEQ ID NO: 149 - protein, 133 aa, synthetic construct
MELDTNAHSVFSLYYHLILVTKYRKKVFDDEVSNRARELFEAIAPNYKIEVLEWNHEQDYIHVFFKAQPKTELSKFINAYKSASSRVLKKEYPSMKKQLWKEMFWSRSFCLLSSGGAPSEVIEQYIEEQGQKK

SEQ ID NO: 150 - protein, 142 aa, synthetic construct
MLIHNRSNVVDFNFHLVLVTKYRKPIFSNATYQQGIRTILQSIADKNDIIIQTMEVMPDHVHLLISFPPKMASSSAVKSLKGSSGREWFKQFPETEAVLWKKHLWTGSFFMSTVGNVSKNIVEAYIKNQKQRPIGRPKKSSH

SEQ ID NO: 151 - protein, 138 aa, synthetic construct
MTVQTLSTLHHCVYNLNYHLVLVTKYRRRCMTGPMLDRLEAICRSTAAKWECEVLEFNGEADHVHLLLALTPKVLPSAFVNNLKTVTSRLLRKEFADHLKQYYWSKPVFWSRSYCILTVGGAPLSVLKQYIEQQERPE

SEQ ID NO: 152 - protein, 130 aa, synthetic construct
MTSGYRRGRHSVSDLKAHLVFVPKYRRNTFTAESLDLIRSAFEGVADKMGFQVLEFGGEADHIHVLLEYPPKLSISQLANHLKGVSSRLYRKQYPSPHADHLWSPSYFAVSVGGAPLEIIKQYINSQKPS

SEQ ID NO: 153 - protein, 133 aa, synthetic construct
MNGKRWKCSATTVYNISYHFIWCPKYRRKVLAGKVAERLKELLLQKAKEIGVEIAQMEIMLDHVHLFVKADPRNSPHFVAQQLKGYTSRVLREEFPVLRSRLPSLWTRSYYCESVGHISEETIRKYIEDQKGK

SEQ ID NO: 154 - protein, 129 aa, synthetic construct
MEYQRDEHRVHLIVYHLVWTPKRRKAVLTGAIGLDCRALIEAKCTEKGWEIIELAIQPDHVHLFVRVWPSVSAAEVVKACKGVTSYELRKKYPTLLKLPSLWTRSYFAATAGNVSSETVKRSIEAQKGL

SEQ ID NO: 155 - protein, 133 aa, synthetic construct
MNNERWKHSNTTIFNISYHIIWCPKYRRKVLVNEVEDRLKTLLVEKAKEIGCEISIQEVMPDHVHIFIKTKPTLAPHYIVQQLKGYTSRILRKEFPKLKSRIPTLWTRSYYCESIGHISEETVKKYINDQKNV

SEQ ID NO: 156 - protein, 137 aa, synthetic construct
MSGDQVLKRGRHCVFKMHVHLVFVTKCRRGVFDGDAISRLRTIFARVCADFEAQLIEMDGEDDHVHLLVEYPPKVAVSNLVNSLKGVSSRLLRKERPDIQKRHWKGVLWSPSYFASSCGGAPIFILRQYIEQQQTPH

SEQ ID NO: 157 - protein, 143 aa, synthetic construct
MIVVYRPSVQTNMGQDYRHANTSVTLINYHFVWIPRRRKPVLVGEVARRFEELLRAKVVELDCLLIALEVMPDHVHLFLNCPPTVAPSNIMHRLKGATAYKLRNEFPHLKKLPSMWTRSFFCSTAGNVSSETVKRYIENQKTR

SEQ ID NO: 158 - protein, 134 aa, synthetic construct
MQEMTDFKSNRNIVYSCTYHVVFCPKYRRPVLTNGVDERLKQIVREILADLECGLLELEIMPDHVHLLVGVDPQFGIHKVIKRIKGQSSRLLRQEFPWLKSRLPSLWTNSYFVATVGGAPIQVVKQYIENQKSH

SEQ ID NO: 159 - protein, 130 aa, synthetic construct
MELKSNKNIIYNCQYHVVWCVKYRRKVLIGDVEARLKEILAQVVVDVRCELDEMETDKDHVHLLISCDPQFGIHRVVKRMKGRSSKLLRDEFPALKSRIPTLWTNSYFVATVGGAPLAVIKQYIKDQQLV

SEQ ID NO: 160 - protein, 143 aa, synthetic construct; identical to SEQ ID NO: 157

SEQ ID NO: 161 - protein, 132 aa, synthetic construct
MEFRRDRNSVSSLQAHLVFVTKYRRKVLTQESLRLLEESFQGVAKKMDFEVLEFNGETDHIHLLIAYPPKWSISQIVNALKGVSSRKYGQAKFPKPYGKTALWSPIYFVSSVGGAPLKVLVAYIQNQEKPPY

SEQ ID NO: 162 - protein, 145 aa, synthetic construct
MSVLEGESLEWMRLRSTRHARYWCSYHFVWIPKYRRDILVGDVAEYTKEVLSEILKELECEPLALEVLPDYVHVFALCPPRHSPAYIVNYLKGKSARKILQRFPELRTGAVRGKLWSRSYFVTTVGNMTADIVKRYVEEQWGKER

SEQ ID NO: 163 - protein, 132 aa, synthetic construct
MKRQFKSNNNIVYSCKYHVVWCPKYRRSVLKDGVDVRLKEILFEVAREKRAEIIELEVMPDHVHLLVDVDPQFGIHRLVKLMKGRSSCILRQEFPWLRSRLPTLWTNSYFVATVGGTPLATLHEYIEKQKYV

SEQ ID NO: 164 - protein, 158 aa, synthetic construct
MKALLPAGDANGTNSCHHAIYLLHTACMKDLRSNNHVVYICRYHVVFCPKYRRQVLTPPIDERLKAILAEQIESCGQELIEMEVLPDHVHLLVGCDPQFGIHRLLKLLKGYSSPALRAGFPVLKRRLPSLWTNSYFVSTVGSVTLETLKRYVESQKER

SEQ ID NO: 165 - protein, 134 aa, synthetic construct
MSEARYRKNAGCVYNLHLHFVWCPKYRKKVLQGKVAERLKELLHEKAAALNTEVEGLEIMPDHVHLFVSVPPTAAPQYFANQFKGYTSRVLRREFSHLRTGLPCLWSRSYYVGAVGHVSAETVQKYIETQKTRS

SEQ ID NO: 166 - protein, 132 aa, synthetic construct
MEFRRDRNSVSSLQAHLVFVTKYRRKVLTQESLRLLEESFQGVAKKMDFEVLEFNGEIDHIHLLIVYPPKWSISQIVNALKGVSSRKYGQAKFPKPYGKTALWSPIYFVSSVGGAPLKVLVAYIQNQEKPPY

SEQ ID NO: 167 - protein, 146 aa, synthetic construct
MKHDFVSSGRAVSDLKAHLVLTTKYRRKVFTSEMLQRLHEVMYDLCQKWGCKLIEFNGEADHVHLLFQYYPQVDLSKLVNNIKSVSSRRMRSEFADYISKIYRKNVLWNESYFIASCGGVTVSALSQYIESQGTPASHPGPSAPRR

SEQ ID NO: 168 - protein, 136 aa, synthetic construct
MRKTNLKKSRTCVYNCNYHVVFSTKYRAKVLNVEIENRLREIINEIANKKGFYVSQVEVGEQDHVHLFLSAHPKKSPSYIVKMIKGISARKLLIEFPQIKEKLWKGHLWNPSYYIETVGSISEKAIKKYIEEQING

SEQ ID NO: 169 - protein, 137 aa, synthetic construct
MKAIYDTNNHSVFLLQYHLILVTKYRRQVIDNEICKRLKEIFEYIAIKDQYKLKIIEFNHDKDHLHILFKAEPKSEISKFINAYKSASSRLIKKEFPKVKEKLWKEYFWSRSFFLATTGGVTLEVLKQYVESQGKKR

SEQ ID NO: 170 - protein, 134 aa, synthetic construct
MKIEKGRGYVYALQYHIVWCVKYRHQILIGEIDTKLQEMLKAIALDNGFEIVEMETDKDHVHLLVSCKPQHYIPSIIKALKGNSARFMFKEFPELKKQLWGGHLWNPSYFITTIGDTNEEQIKHYIQSQKVKHL

SEQ ID NO: 171 - protein, 134 aa, synthetic construct
MKTQKERHATHLLHAHLVFVTKYRYKVLQGKHIEYLRKIFKETIEEMGGTLEEFDGEADHVHLLIQYPPKWSISKIVNNLKGRSSRLLRRDMPDIKESYWGKAGLWHRSYFAGSVGGAPLEIVKQYIEQQKTPD

SEQ ID NO: 172 - protein, 135 aa, synthetic construct
MDIQKSRHATHLLHAHLVFVTKYRYNTLTKEHIDYMGKIFKETIEEMGGTLEEFDGENNHVHLLIQYPPKHSISVIVNNLKGRSSRLLRRDMPNIKERYWGKGSALWHRSYFAGSVGGAPLEIVKQYIQQQDTPY

SEQ ID NO: 173 - protein, 135 aa, synthetic construct
MRLEKNRHAVYKLQFRLIIVTKGGHPIIDKEISLRLKEIAKHIFEDSWKCKILEVETEEDHIHIAFESSPQTQLSKLVNNFKTVSSRLIRKEFKNHLRKYYWKDFFWSPSYCLLSVGGAPIEIIREYIRSQKKPQ

SEQ ID NO: 174 - protein, 133 aa, synthetic construct
MSSLIHDRTTVHNIGYHIVWSVKYRRKVLVGDVETSLKDILYQIAGEKGFIIKEMKCDIDHIHVFASAKPKVSPSYIYKMMKGISARRLFVRHPELKSKLWGGHLWNPSTYIETIGHISEDTVRKYIEDQKKK

SEQ ID NO: 175 - protein, 137 aa, synthetic construct
MKYELKSTRHAKYLLHAHIVFTPKYRKKIFTKEHLDLMKTVFEDICKMNDATLEEFDGEADHVHLLVLYPPRIALSVLVNSLKGVSSRRLRQNFDIFKKEYWGENVALWSRSYFVASVGGAPIEILKQYIEQQKTPD

SEQ ID NO: 176 - protein, 138 aa, synthetic construct
MRNNTDIRHGRHCVFNLHVHLVFVTKYRRGVFTKEVINDLREIFTGVCADFEAELVEFDGEDDHVHLLVNYPPKVAVSALVNSLKGVSSRMIRKKNYPEIKKKLWGAALWSPSYFAGSCGGAPISIIPQYIEQQRTPE

SEQ ID NO: 177 - protein, 138 aa, synthetic construct; identical to SEQ ID NO: 176

SEQ ID NO: 178 - protein, 138 aa, synthetic construct; identical to SEQ ID NO: 176

SEQ ID NO: 179 - protein, 128 aa, synthetic construct
MTNKTDLNYHLIIVTKYRKPILRPDIMEYTISIIEQELKKIHCEIIAINGDEKNHIHILLKIKPMQSISLIVKIIKQYSTYHVWQKFGVDLRRDYWYNNILWSSGYFCCSIGNASEETIKNYIENQGN

SEQ ID NO: 180 - protein, 136 aa, synthetic construct
MTTKYKWDKNKHSVYALHYHLIICVKYRRKAFEHDDVAARLKEMSINISKNFDVQITNQEVDKDHIHILFKCKPQTDLIKFINSLKGATSRVLRNEFPKLKNILWGDSFWSNSYCLITTGQVSLDQLIKYVQSQGE

SEQ ID NO: 181 - protein, 137 aa, synthetic construct
MSKKSNYNSTNRSKHYLKCYLIFVCKYRKKLLVGQLKDDMKQLLLNITSKSDFEIEVFESDIDHIHFLIRYIPRLSITSIVRKLKQESTYHIWRSTYRNYLFNHFWKEKTFWSDGYFVCSIGEANPNTIKQYILTQG

SEQ ID NO: 182 - protein, 126 aa, synthetic construct
MKNKRTKHSAYNLNYHIVFVTKYRHQVLKDKIQDCVKIRMQELCKEYGWEMLALEVMPDHIHLFVSAEPKIAPLAIATTLKSILTVDIFKKFKTLKSKYFWDQVCSVEGVTMDQQAQFPQQLLNNT

SEQ ID NO: 183 - protein, 136 aa, synthetic construct
MKLKSIDYKHYNHSVGLACVHLVWIPKRRKRVLVGNVATRCREIFYELAIEKEWEIKALEIAPDHIHLFICHQPNYSISQIVQAFKGRSSCYLRREFPDLLKLPSLWTRSYFHTTAGSVSSKVIMEYINDPHHSTH

SEQ ID NO: 184 - protein, 134 aa, synthetic construct
MIYKLDKNCHSVYTLNYHLIFCVKYRRKIFLNEKIINKTKELMKNIALGFDVELINQELDKDHIHILFKAKPQTNLVKLINSLKGASARVLFKEFPEIKKKLWNGHLWSASYCLITTGQVTLDVLKKYVESQGK

SEQ ID NO: 185 - protein, 103 aa, synthetic construct
MAPEIQRKLKGVVLEVCKERLATVISMEMMPDHVHLLVEIDPQYGIHKLVKQIKGRSSRLLRKQFVQLTTRLPTLWTNSYFVSTVGGAPLSIIKKYVEEQKGI

SEQ ID NO: 186 - protein, 139 aa, synthetic construct
MARNWERTDYRRGRTVVSLLHVHLVFVTKYRRGVLSERAIEVIHEAMNSVCVDFGAKLVELNGEDDHVHALVEYPPTIALSKLVNSLKGVSSRILRSKEFPEVQAKLYGEHLWSPSYFAVSCGGAPLEIVRQYIEDQRK

SEQ ID NO: 187 - protein, 140 aa, synthetic construct
MKNNQRKLYGYQATNRAKHNLKVHLILVCKYRKKLLVGNINKLIKNTIYEIESKSDFNIIEMETDKDHIHIMLQYIPRISISSIVNRIKSITTHRIWKNHNIYLQKHFWRENTFWSDGYFACSIGEASPETIRNYIANQG

SEQ ID NO: 188 - protein, 142 aa, synthetic construct
MNDTVYFSIPNDVTHGRGYVYSLQYHIVWTTKYRKPIFIGAIEAEVKQYLLETLKSLDVDVLALEIMPDHIHLLVDCKPQLRLSDAIKILKGNTARWLFLKHPEIKKQLWGGHLWNPSYFVATVSDRTLEQVAQYINSQKTK

SEQ ID NO: 189 - protein, 157 aa, synthetic construct
MEQNILVLDLKIAYNTYMNITYKSNNNVVYSCKYHVVWCPKYRRKILTNGIDTRLKELLLEYAANISVDIMEMEIMPDHVHILMEVDPQFGIHKAVKSLKGYTSKVLRSEYPSLKTRMPSLWTNSYFVSTVGGAPLDVIKQYIENQKTSQRQKDKLG

SEQ ID NO: 190 - protein, 134 aa, synthetic construct
MINNNYNTSAHLTYLCRYHVVFCPKYRRKVLTDGKDARLKEVFMGTAKAHSFEILEMKVMPYRGLHLLISCNPRYGIMECVKNLKYESAHVLLREFPDIKRRLPAMWTRSAFIATVGSVSLDVVKQYIKDQKGK

SEQ ID NO: 191 - protein, 138 aa, synthetic construct
MEYKSNRTIVYSCRYHVVFCSKYRRKLLADGVDIRLKELTAQVCEKENIELLEMEVMPDHVHLQLEVDPQYGINSAVKQIKGVTSHVLRSEFPWIKSKVPTLWTNSYFVSTEESVPLFEVRQYIESQKTSQRQKEKMG

SEQ ID NO: 192 - protein, 106 aa, synthetic construct
MLNAEVEQRLREILADIASNREYAIQKMEIMPDHVHVFASAHPKIAPGYIYKTLKGISGRKILFEFPEVKQKLWKGKLWNPATYIETIGHISENTVLKYIEDQKQK

SEQ ID NO: 193 - protein, 132 aa, synthetic construct
MAVTLRSNSNIVFQCAYHVVWCPKYRRRVLGGRVEERLKDLIREVVDEKGAWLVEMEVMPDHVHLLVEVDPQYGIHRLVKAIKGRSSRVLREEFPHLKSRLPTLWTNSYFVATVGGAPLAAVKRYVEQQKGR

SEQ ID NO: 194 - protein, 134 aa, synthetic construct
MRVKERLKSNNNVVYSCKYHVIWCPKYRRPVLVDAVEKRLKELIHQQASRFAADLIEMEIMPDHVHLLIEIDPQFGIHRLVRNVKGYTSRILRSEFKTLKTRIPTLWTNSYCVITVGGAPLSVIKQYIENQKTV

SEQ ID NO: 195 - protein, 136 aa, synthetic construct
MRTSAGAAYDLGYHVVWCPKYRRPVLDGRVKDRLEELIRAKADEHGWEIVALEVMPDHVHLFVKAHPKHSPSYVANQFKGFTSHHLRTEFPRLRSRLPTLWSRSYFVATVGSVSAETVQRYIETQYERPSKGGGRA

SEQ ID NO: 196 - protein, 131 aa, synthetic construct
MQYHKNRHAAYLLHAHIVFVTKYRRKILGDLHHQAFSRTAASVCRDFGATLRDCNGESDHVHLLIDYPPSVQLTRLINSLKAVTSRRMRREFSDLAGAYSKPVLWSRSYFVSSCGGAPLEVIKSYIQNQQG

SEQ ID NO: 197 - protein, 137 aa, synthetic construct
MMKNTDIRTGRHCTFLMHVHLVFVPKYRKNVFDRDALSRLEGIFRHVLDQFEAELAEFNGEPDHVHLLVNYHPKVAVSRMVNSLKGVSSRKLKQQRPDLAKIYWKGTLWSGSYFAASCGGAPLDTIRQYIEEQQKPT

SEQ ID NO: 198 - protein, 135 aa, synthetic construct
MASNIRRGRSVVSDLQAHLVFITKYRRKVISKRVLRRLREIFDSVCCHFDATLLECDGERDHVHLLVAYPPKYPVSKLVNSLKLVSSRLIRKEGFHEVEANLYGKHFWSPSYYAGSCGGANLQTVAKYIQNQSSP

SEQ ID NO: 199 - protein, 133 aa, synthetic construct
MLKESEYKSGHHCKYLLNLHFVWCPKRRKKVLIEGVARRAAEIFHILAVEKGWDILALEIAPDHIHLFVSVPPTESPHLVIKAFKGRSSFYLRKEFPELLKLPSLWSRSYFVSSAGNVSSAVIKRYIEDPHHG

SEQ ID NO: 200 - protein, 136 aa, synthetic construct
MYRKERHSVTDLKVHLVCVTKDRKPVLIEDGLKLIEQSFGEVALKMHFNILEFNGEADHVHALIEYPPKLSLSAITNALKGVSSRRYGTAGYPKPHKESLWSPSYFAVSVGGAPIEVLKQYIKDQSRPKTAGLESH

SEQ ID NO: 201 - protein, 135 aa, synthetic construct
MQPRKGSHSVFSVQLHMVFVTRYRRKVITSVMLERLREIFANICLKTKCHLTEFSGEADHVHLLVDFHPDNRLSTLIGSMKSGSSRIIQKEFADDLSKTYKKPAFWSGSYYVASIGGAPIERVKAYIKSQEIPEK

SEQ ID NO: 202 - protein, 132 aa, synthetic construct
MGYFSKKRRKYYLKCHLIFVCKYRKRLITDEVREYILNVFKSIEEQSDFDIEVMETDKDHIHMLISYPPNISVSSIVRRLKQCSTLSLWKQFPIMLSKQFCKECTFWSDGYFVCSIGEANPDTVRKYIENQG

SEQ ID NO: 203 - protein, 133 aa, synthetic construct
MEVTHGRGYVYSIQYHIVWCVKYRHKIITEKIENRLIEILNKIADDNGFQILECNTDKDHIHLLVNCSPQHYIPDMIKALKGVSARLLMKEFGEELKRKLWGGHLWNPSYFVATVSENTEEQIRKYIQNQKRK

SEQ ID NO: 204 - protein, 141 aa, synthetic construct
MQKKERVPGAIHERNYIYNFHFHLIWVTKYRNPAFTTQDLVDDMKAILTQLAKEKGITIEEMEVMPDHIHLLISFKPKYAPADIVKYLKGHSAKQFFMMHPEIRDQEFWCGHLWSHSYFMSTLGDMSREVVEKYIRNQYSK

SEQ ID NO: 205 - protein, 134 aa, synthetic construct
MELKCNRNCHSKYRLEYHLILVTKYRKPCITPEMLQYLRRECQRLLELNGIELLEMNGEADHIHLLFSAPPQVCLANVINSLKSSTSRLVRKTYAEHLAQFYWKPYFWSRSYLILSSGGAPIEVIRKYIQEQGK

SEQ ID NO: 206 - protein, 137 aa, synthetic construct
MSYRTNRHSCYDLEYHLVVVTKYRHPVIDGAFKDRLTELTYRIFEENFQCYVNEVNTDKDHIHILFEAPPQVALSSLVCSYKTVTARLLRKEFAEYLAPFYWKPYLWSRSYFVCTVSERSHELITEYIRNQENKEKG

SEQ ID NO: 207 - protein, 113 aa, synthetic construct
MLKDGIDTECKEMLQDLAAEYNFQILAMEVMPDHIHLLVDCKPQFYISDMIKIMKGNLARQMFLVHPELKKELWGGHLWNPSYCAVTVSDRSRDQVLTYIEGQKEKEKRRVRT

SEQ ID NO: 208 - protein, 133 aa, synthetic construct
MEDKYRHTNTTVSLINYHFVICPRYRRKIFLIPHVEERFKELVKIKCEELEIKIIKVECDKDHSHMFLNCLPTLSPSDIMQQIKGYTSKILREEFVELSKMPSLWTRNYFVSTAGNVSSEIIKKYVENQKKRY

SEQ ID NO: 209 - protein, 136 aa, synthetic construct
MSNTYKSGSHSKYLLQYHLIFVCKYRRKILSSPNIASDIKRLSIEYCQKHNVVIHYMETDKDHIHYMIETTPNINLSNMVKTMKSYITYHIWEKYSAYLSKCFWKERTFFSDGYFISSIGNVSQETLKNYIENQGK

SEQ ID NO: 210 - protein, 146 aa, synthetic construct
MKLDNNNHSVFLLNYQLIMCTKRRNKVIDDVISNRLREMFENLSIKYNISLDEWNHDADHVHILFRGQPNSEISKFINAYKSASSRLIKKEFPEIRQSLWKEMFWSPSFCLLTTGGATVEIIQQYIQSQGGKDGKQSNKVQTVSKR

SEQ ID NO: 211 - protein, 130 aa, synthetic construct
MELDTNAHSVFSLYYHLILVTKYRKKVFDDEVSNRARELFEAISPNYKIEVLEWNHDQDHIHVLFKAQPKTELSKFINAYKSASSRVLKKEYPSMKEQLWKEMFWSRSFCLLSSGGAPIDVIKQYIEEQG

SEQ ID NO: 212 - protein, 133 aa, synthetic construct
MEVVKGRGYVYSIQYHIVWCVKYRRNILNGQIETRLKELLTQISKDNGFQILEMNIEEDHIRMLINCTPQHYIPNIIKAMKGVSARLLMKEFGDDLRSKLWGGHLWNPSYFVATVSENTEEQIIEYIRSQKRK

SEQ ID NO: 213 - protein, 134 aa, synthetic construct
MPSDVTHGRGYVYSLQYHIVWVTKYRKPVFTGKIESDVREYLNETLKTLDMDVIAMEVMPDYIHLLVNCKPQLRLSDAIKILKGNTARWLFLSHPEIKNQLWGGHLWNPSYFVATVSDRSLKQVTEYINSQKTK

SEQ ID NO: 214 - protein, 149 aa, synthetic construct
MYVNILNIHIICYTIRMEDKYRHTNTTVSLINYHFVICPRYRRKIFLIPHVEERFKELVKIKCEELEIKIIKVECDRDHSHMFLNCLPTLSPSDIMQQIKGYTSKILREEFVELSKMPSLWTRNYFVSTAGNVSSEIIKKYVENQKKRY

SEQ ID NO: 215 - protein, 127 aa, synthetic construct
MYSLQYHLVWCTKYRKQVLKNGIDAECKKMLYGLAEEYKFKILAMEVMPDHIHLLLDCKPQFFISDMIKIMKGNLARQLFLSHPELKQELWGGHLWNPSYCAVTVSDRSRDQVVAYIEGQKEKEKRK

SEQ ID NO: 216 - protein, 145 aa, synthetic construct
MFKFENEVSKPIKERSYVYDLHYHVVWVTKYRVHAFNTDKVVNALKNKLLEIAKDNGYTIEAMEIMPNHVHLLISAKPKVSVTVMVKKLKGITGKWLFQAFPDEMRKYFWHSHIWSPSYYVGSVGNTTEAIVKKYIETQKERSFK

SEQ ID NO: 217 - protein, 122 aa, synthetic construct
MYVHLVFVTKYRKAIFKAQHLKAMQEVAAKVCLDFGAELRELDGERDHVHLLVSYPPKVAVSSLVNSLKGVTSRMLRKQFPDLEAAYYKGQLWSPSYFAASCGGAPLEKIKEYVRQQDTPSK

SEQ ID NO: 218 - protein, 144 aa, synthetic construct
MRLVLDSCQWYNTIMEFKSNSTVVYSCQYHVVWCPKYRRKVLVNGVDTRLKEIIEEVCKEYHIDLIEMEIAPNHVHLLLEVDPQFGIHKAVKQIKRQSSKILREEFTWLNSRLPTLWTNAYFVATVGKNPDMAIAQYIEAQKDV

SEQ ID NO: 219 - protein, 135 aa, synthetic construct
MQGTHYNRKNRRKYLLKAHLVLVTKYRKTLLTGDIDKTIKGCIADTAKNNGREIVAMETDKDHVHILLSYDATDRICDIVKVIKQTTTYQLWANYERLLKKHYWKQRIFWSDGYFACSIGDVSSATIEKYIEEQG

SEQ ID NO: 220 - protein, 146 aa, synthetic construct
MKLDNNNHSVFLLNYQLIMCTKHRNKVIDDVISNRLREMFENLSVRYNISLDEWNHDADHVQILFRGQPNSEISKFINAYKSASSRLIKKEFPEIRKSLWKEMFWSPSFCLLTTGNATVDIIQQFIQSQGGKDGKQSNKVQTVSKR

SEQ ID NO: 221 - protein, 136 aa, synthetic construct; identical to SEQ ID NO: 9

SEQ ID NO: 222 - protein, 134 aa, synthetic construct
MNIVHGRGYVYGLQYHIVFCVKYRKPILTGEAETRLRDIFSMIAEDNDFTIEEMNMDKDHVHLLVSCSPQNYIPSMMKALKGVSAHKLFKQMPELKKELYKGHLWNPSYFVATVSENTEAQIRKYIQSQKVVNV

SEQ ID NO: 223 - protein, 136 aa, synthetic construct
MNTEYKTNRHSCYSLEYHLVVVTKYRHSVIVTELKERLLALSKEVIEDFWKCKITAINTDKDHIHIMFEAPPQVQLSKLINNYKTVTSRRIRKEFADFLKPYYQKPYFWSDSYFVCTVSDRSHEMIQRYIAAQGIK

SEQ ID NO: 224 - protein, 139 aa, synthetic construct
MKIQANRHSIYELQYHLVVVTKYRKKIINDKIKDELTDIAINIFENSYNGKIVEINTNKDYIHILFSVGPQVQLSKAINSFKTVSSRLVRKNNADYLNAFYSKPYFWSRSYYIGSVGNVNVETIKNYIRNQGKKAPNPF

SEQ ID NO: 225 - protein, 140 aa, synthetic construct
MKKKTGYEYYTNRHSCFLLQYHIVLITKYRHPVLTGNVKTAVYERINYVAESRGFNILELNGELDHIHLLIEANPQTPPSELVNVIKTQTSRKVRKLYGDTLLKKYYWKPYFWSDSYFIATVSENTLDVVKNYIKNQGIK

SEQ ID NO: 226 - protein, 135 aa, synthetic construct
MIQKDYISKNHSKFLIKYHVIFVCKYRKKLLIGAVEYDMKQIMQNISDLSDFNIEVMETDKDHIHMMICSEPKLSPLQIVRRLKQMSTTAIWKRHENYLRHVFYWENTFWTDGYFVSSVGNVSQETIKKYIENQG

SEQ ID NO: 227 - protein, 155 aa, synthetic construct
MKEYIRTRHSVYLLTYHMVFVTKWRKPVITDEIGDFMVATAKRLCDGYGGELISGETDRDHIHLLVSLPPSKNITDIVRSLKTQLSKEVHAHPEYDRIVKKHIYGNAPLWSPSYFVTTTGSVSMDVVKQYIEGQRTDEHKRKYEKRSLDYWNSRL

SEQ ID NO: 228 - protein, 117 aa, synthetic construct
MTIETHHGRGYVYCLHYHIVWCVKYRHKVLTPEIVEDLVQILEKIANDNDFEILELNIDKDHVHILIDCSPQHFIPDIMKAIKGVSARLLMKKHGDTLRKKLWGAFVEPVLFYNECF

SEQ ID NO: 229 - protein, 144 aa, synthetic construct
MADNADIRTGRQAIWDLHVQLVFVTKYRRYVFTDDMLKTCENIMRDVCKNFGATLEEFSGGANHVYLLVGYPATVAVSKLVNSLKGVSSRILRRDYADEIAPYLLDGHLWSRSYYAGSASGALSQAALTGYTGINSPAVLACLG

SEQ ID NO: 230 - protein, 141 aa, synthetic construct
MLDAVEWYNKSVEYKSNHNVVYSCKYHVVWCPKYRRKVLVNGVDIRLKKLIEEICGEYHIDIIEMEIMPDHIHLLMEVDPQFGIHKAVKLIKGRTSRVLRQEYGWLRSRLPSLWTNSYFVSTVGGAPLSVIKKYIENQKNV

SEQ ID NO: 231 - protein, 138 aa, synthetic construct
MYKKRSGQTYYTNRHSCFLLQYHIVLVTKYRRPVLTGPVKDLVYQVIQDIFDEKKLVILEMNGEADHIHILFEADPFTAPGTLVNIIKTKTSRYARKEYGETLLKEYYWKPLFWSDSYFVTTVSENSLQMVQEYIKNQ

SEQ ID NO: 232 - protein, 130 aa, synthetic construct
MMYKSNKNIVYSCKYHVVWCTKYRRKVLINGADKALKKTAYQVAQELQFEILEVEVMPDHVHMLIEVDPQLGIHRAVKRIKGRTSHELRDSFEWLRTKLPTLWTNSYFVSTVGGAPLSAIKTYIENQKNV

SEQ ID NO: 233 - protein, 140 aa, synthetic construct
MRKKDNTDYYINRHSCFLLQYHIVFVTKYRHPVLTDKVKDVVYERIHYVADQRGFHILEINGESDHVHILMEADTTTSPAELANVLKTQTARKARKLYGETILKKYYWKPYFWSDSYFISSVSENSLHTIKQYIRNQGKR

SEQ ID NO: 234 - protein, 140 aa, synthetic construct
MKKKTGSDYYTNRHSCFLLQYHIVLVTKYRHPVLAGGVKEAVYERIRYVAESRGFNILELNGEADHVHLLIEAEPQTSPGELVNVIKTQTSRRARKLYGDTELRPYYWKPYFWSDSYFIATVSENTLEVVKKYIKDQGIK

SEQ ID NO: 235 - protein, 144 aa, synthetic construct; identical to SEQ ID NO: 60

SEQ ID NO: 236 - protein, 149 aa, synthetic construct; identical to SEQ ID NO: 112

SEQ ID NO: 237 - protein, 155 aa, synthetic construct
MDDFNRKRHAVYKLTYHAVFVIKYRRKVMSEPIIEHMRQYAAHLIGQCYQGRLIELNGEPDHIHILFELPATAAPSVVVCSLKTQLSKEIRANFWDEIKDKLWNNIFWSDSYFITTTGGADIETIGKYIQDQGIEKPKRKYTKRQTKKCEKKTRT

SEQ ID NO: 238 - protein, 134 aa, synthetic construct; identical to SEQ ID NO: 129

SEQ ID NO: 239 - protein, 131 aa, synthetic construct
MKLDNNNHSVFLLYYHLVLVVKCRRKVFSDQMSQYAKDIFVRIGSSYNITLEEWNHDQDHVHIMFRAHPNTEMSKFINAYKSASSRLIKKDFPEVRRKLWKEMFWSRSYCLLTTGGAPIETIRKYIENQGR

SEQ ID NO: 240 - protein, 139 aa, synthetic construct; identical to SEQ ID NO: 128

SEQ ID NO: 241 - protein, 130 aa, synthetic construct
MKYKSNNNVIYSCKYHVVFCPKYRRPVLVDGVDDRLKELIKETCDQLNVEIIEMEIMPDHVHLLMEVDPQYGIHKAVKRIKGYSSRVLRQEFSWLKSRIPTLWTNSYFVSTVGGAPLSVIKQYIEDQKNV

SEQ ID NO: 242 - protein, 135 aa, synthetic construct
MIFDTNNHSVFVLNYHLVMCVKYRREVITDSISNRLNAIFEKIAPSYHITVEEWNHDRDHIHVLFRGQPNTEISKFINAYKSASSRLIKKEFPEIKESLWKEMFWSQSYCLMTTGGVTAEVIRQYIQSQGKKDGK

SEQ ID NO: 243 - protein, 133 aa, synthetic construct
MKNEYKHTKTTVSLINYHLVFCPRYRRKIFNIPGVEERFKELTSAECFNHEIDILAMECHVDHVHMFVSVYPQQSIPDVVKQIKGATSHKLREEFPQLKNMPSLWTRSYFVSTAGSVSSETIEWYINTQKTRD

SEQ ID NO: 244 - protein, 141 aa, synthetic construct
MLDAVEWYNKSMEYKSNHNVVYSCKYHVVWCPKYRRKVLINGVDIRLKKLIEEICGEYHIDIIEMEIMPDHIHLLMEVDPQFGIHKAVKLIKGRTSRVLRQEYGWLRSRIPSLWTNSYFVSTVGEAPLSVIKQYIENQKNV

SEQ ID NO: 245 - protein, 118 aa, synthetic construct
MVFVTKYRRSVFTKAVLEDLKEIFASVCQDFEAELVEFDGEKDHVHLLINYPPKVSVSRLVNSLKGVSSRLIRKKRYPCMQDKLWAGALWSPSYFAASCGGAPISIIKQYIEQQDTPE

SEQ ID NO: 246 - protein, 151 aa, synthetic construct
MKDIHSLSHTKWNCKYHVVFAPKYRRKVFYGEKKVEIGKILRELCKWKGVNIIEAEVCPDHVHMLVEIPPKMAVSWFMGFLKGKSSLMIYEKWGNLKYKYRGREFWCRGYYVDTVGKNTKAIAEYIKNQLKEDELNQQLVLDYGDPFTGDK

SEQ ID NO: 247 - protein, 131 aa, synthetic construct; identical to SEQ ID NO: 102

SEQ ID NO: 248 - protein, 155 aa, synthetic construct
MKEYIRTRHSVYLLTYHMVFVTKWRKPVITDEIGDFMVATAKRLCDGYGGELISGETDRDHIHLLVSLPPSKNITDIVRSLKTQLSKEVHAHPEYDRIVKKHIYGNAPLWSPSYFVATTGSVSMDVVKQYIEGQRTDEHKRKYEKRSLDYWNSRL

SEQ ID NO: 249 - protein, 132 aa, synthetic construct
MDERWKTNPGCVYNVSYHIIWCTKYRRKLLLGEVEERLRSLLIEKATANGWDIPELEIMPDHVHLFVKATPSDSIAHITSQLKGYTSFSLRNEFPHLKQRVPTLWTRSYYVETIGHISEKTILKYIEDQKKK

SEQ ID NO: 250 - protein, 131 aa, synthetic construct
MKLDNNNHSVFLLYYHLILVVKYRRKVFSDQMSQYAKDIFVRIGSSYNVTLEEWNHDQDHVHIMFRAHHNTEMSKFINAYKSASSRLIKKDFPEVRRKLWKEMFWSRSYCLLTTGGAPIETIRKYIENQGR

SEQ ID NO: 251 - protein, 138 aa, synthetic construct; identical to SEQ ID NO: 85

SEQ ID NO: 252 - protein, 133 aa, synthetic construct
MEVVKGRGYVYSIQYHIVWCVKYRRDVLNGQVATKLKELLTQISKDNGFQILEMNMDEDHICMLINCTPQHYIPNIIKAMKGVSARLLMKEFGDDLRSKLWGGHLWNPSYFVATVSENTEEQIIEYIRSQKRK

SEQ ID NO: 253 - protein, 130 aa, synthetic construct
MEYKSNHNIVYSCKYHVVWCPKYRRSVLINGVDVRLKELIKETCDSLNVEIIEMEIMPDHVHLLMEVDPQFGVHRAVKQIKGYSSKVLRTEFPWLKSRIPSLWTNSYFVSTVGGAPLAVIKQYIENQKSV

SEQ ID NO: 254 - protein, 157 aa, synthetic construct
MEQNILVLDLKMAYNTYMNITYKSNNNVVYSCKYHVVWCPKYRRKILTNGIDTRLKELLLEYAANISVDIMEMEIMPDHVHILMEVDPQFGIHKAVKSLKGYTSKVLRSEYPSLKTRMPSLWTNSYFVSTVGGAPLDVIKQYIENQKTSQRQKDKLG

SEQ ID NO: 255 - protein, 135 aa, synthetic construct
MKKKTGSDYYTNRHSCFLLQYHIVLVTKYRHPVLAGGVKEAVYERIRYVAESRGFNILELNGEADHVHLLIEADPQTSPGELVNVIKTQTSRRARKLYGDTELRPYFWSDSYFIATVSENTLEVVKKYIKDQGIK

SEQ ID NO: 256 - protein, 130 aa, synthetic construct
MKYKSNRNIVFSCSYHVVFCPKYRRPVLVDGVDERFKEIAWEVADELGFEIAEMEVMPDHVHLLLSIDPQLSIHRAIKRIKGRSSHDLREEFPWLKSRIPTLWTNSYFVSTVGGAPLAQVRKYIERQKEV

SEQ ID NO: 257 - protein, 134 aa, synthetic construct
MGEWKSKNHSKYLLQYHLIFVCKYRKKLLASNSISNDIKSLSKSICEKHNVIIKYMETDKDHMHYMIETEPNINLCDLVRTVKSYTTYHIWKKYHTYLSKCFWKEHTFWTDGYFVCSVGNVSEKILKEYIENQG

SEQ ID NO: 258 - protein, 138 aa, synthetic construct
MMSRKPYHSASHCKYLIQYHIIWCLKFRFSVLHGKADITLKQILQKICEDYGYKMKALEVMPDHIHIFVDVPQTVAPCDVVRTLKSKSAIELFKAYPTLKRLYARCGVLWSRGYFISTVGQISEATVMQYIEEQKSHE

SEQ ID NO: 259 - protein, 139 aa, synthetic construct; identical to SEQ ID NO: 128

SEQ ID NO: 260 - protein, 133 aa, synthetic construct
MEVVKGRGYVYSIQYHIVWCVKYRRDVLNGQVATRLKELLTQISKDNGFQILEMNIEEDYIRMLINCTPQHYIPNIIKAMKGVSARLLMKEFGDDLRSKLWGGHLWNPSYFVATVSENTEEQIIEYIRSQKRK

SEQ ID NO: 261 - protein, 137 aa, synthetic construct
MLELVFNRNCVYQTAYHVVWCPKYRRSVLTGAVADSLRLLLTEVCNENGWVILSLEIQPDHIHLFVSIPPSKAVSDAVKKLKGTTARKLFLQHPDLKKQLRDGHLWSPSYYVGTAGKVSAETIRQYIERTEHIRGRR

SEQ ID NO: 262 - protein, 134 aa, synthetic construct
MTLANRYTSSSHCVYNLGYHIVFCPKYRRKVLVDGIDERLKELLKEKAQELEITIESIEVMPDHVHLFIRSKPTYAVHFVVNQLKGYSSVKLRKEFPKLKSRLPTLWTRSYFVESVGHISEDTMKKYIENQKNV

SEQ ID NO: 263 - protein, 132 aa, synthetic construct
MNLDTNNHSVFKLSYHLVLVIKYRKKVINDNISNRLREILENISPKYNITLEEWNHDNDHVHLLVRASPNTEISKFINAYKSASSRLIKKEYPIIRKQLWKEYFWSRSYCLLTTGGVTIELVRKYIENQGMK

SEQ ID NO: 264 - protein, 136 aa, synthetic construct
MKENLIYYRTCVCNINYHMVWSVKYRRKILTPEVEKYLQELVQQIADDKGFTVHLFECGEGDHVHCFVSAPPKLSITAIIKYLKGITGRKLFERFPEIRNQLWKGELWNHSYYVETIGSVSEENIRRYIEHQSKSY

SEQ ID NO: 265 - protein, 137 aa, synthetic construct
MSYRTNRHSCYDLEYHLVVVTKYRHPVIDGAFKDRLTELTYRIFEENFQCYVNEINTDKDHIHILFEAPPQMTLSSLVCSYKTVTARLLRKEFAEYLAPFYWKPYLWSRSYFVCTVSERSHELITEYIRNQRNKEKG

SEQ ID NO: 266 - protein, 144 aa, synthetic construct
METRSASSAVYELAYHIVWCPKYRKNLPEPVQRGLKTCIRSVAEARGWEVKELEVMPDHIHLFIGAPPSESPTGIVKVLKGTTSVVMFREHPTLRFTFRHGHLWSPSYYVGSVGHVSEETVARYVREQKLRVAGRPKGRASSPQ

SEQ ID NO: 267 - protein, 147 aa, synthetic construct
MYALSVPTTTPRCLRSRHSGSLLFAHLVVVVKYRRRVFSPALRGQLRALWTEQCEQLGAILVECNGEPDHVHLLLRHPPSLPLWRLVQQLKGVASRTLRQRHPWLRRVGRGHLWSPSYFVVSTGGAPIAVGCRHIDQQGATTALCPD

SEQ ID NO: 268 - protein, 151 aa, synthetic construct
MGSRRALLRTPHVVHVCVYHVVWATKYRRQVLDLRNTPDGPRVCDRLKEIAAGIAAENDWTIRALETMPDHVHLFIQADNVTSPRDLANAFKGRTSHVLREEFPELKRRLPSLWTRSYFVATAGDVSAATIQQYVQNQRTRPTDPKGVGRA

SEQ ID NO: 269 - protein, 137 aa, synthetic construct
MKNKYIHARTCVYNINYHIVWCVKYRRKVLTQEICDRLYEVVMDIAKEKGFSIVECKVGEMDHVHCFVTAPPKLSITQVVKYLKGISGNTLLKEFPELRETLWHGQLWNGSYFCETIGSTSEENILKYIERQKTCQL

SEQ ID NO: 270 - protein, 138 aa, synthetic construct
MALDNDIRRGRRSTYDLHAHLVFVTKYRRQVFTSTHLERLREVFSSVCADAGATLEEFNGEDNHVHLLVNFPPDVPLSRLVNSLKGVGSRRLRSEFPELEKLCWNGKKLWTPGYYAGTTGGAGIDTVRRYIEQQDTPA

SEQ ID NO: 271 - protein, 138 aa, synthetic construct
MLARKRTSVTDFNFHLVLVTKYRKEIFTTREKHDNMIAILKKIAENKEVTISHIEVMPDYVHLMISFPPKLAPSDVVKSLKGTSAREWFKQFPETKKLLWGGHLWTGSFFMSTVGNISKEIVAEYIENQMQKDIKKDR

SEQ ID NO: 272 - protein, 118 aa, synthetic construct
MTIETHHGRGYVYCLHYHIVWCVKYRHNKVLTPEIVEDLVQILEKIANDNDFEILELNIDKDHVHILIDCSPQHFIPDIMKAIKGVSARLLMKKHGDTLRKKLWGAFVEPVLFYNECF

SEQ ID NO: 273 - protein, 133 aa, synthetic construct
MKLDTNAHSVFLLHYHLILVVKYRRQVFTDEISERAKEIFEYIAPSYKIELVEWNHDKDHVHILFKGQPKTEISKFINAYKSASSRLLKKEFPVIRQRLWKEMFWSQSFCLLSSGGAPIDVVKQYIENQGQKK

SEQ ID NO: 274 - protein, 135 aa, synthetic construct
MDRVYHSNHNIVYSCKYHIVFCPKYRRKVLVNGVDVRLKELIHSIADENNFDVIEMEIMPDHVHLLLEVDPQFGIHKAVKTIKGKTSRILRQEFHWLTTKLPTLWTNSYFCSTVGGAPLNIVKQYIESQKTSQRK

SEQ ID NO: 275 - protein, 125 aa, synthetic construct
MYNINYHVVWVVKHRKQILTPGIEQELRQLCEETAAEKDFTLHLFEVQNKDHVHCFVSASPKLSITYIVKMLKGISGRKLLFNHPEIKSQLYKGELWNHSYYVETIGSVSEENIRRYIEKQYNSY

SEQ ID NO: 276 - protein, 133 aa, synthetic construct; identical to SEQ ID NO: 273

SEQ ID NO: 277 - protein, 158 aa, synthetic construct
MFTKNKYIWYNINMEKDIFNISKNELSYGRGYVYSLQYHLIWCTKYRKKVLKDGLDTECKEMLQALAEEYKFQILAMEVMPDHIHLLVNCRPQFYISDMIKIMKGNLARQMFLAHPELKKELWGGHLWNPSYCAVTVSDRSREQVLAYIKGQKEKEKI

SEQ ID NO: 278 - protein, 133 aa, synthetic construct
MKLDTNAHSVFLLHYHLILVVKYRRQVFTDEISERAKEIFSYIAPSYKIELVEWNHDKDHVHILFKGQPKTEMSKFINAYKSASSRLLKKEFPIIRQKLWKEMFWSQSFCLLSSGGAPIEVIKEYIETQGQKK

SEQ ID NO: 279 - protein, 133 aa, synthetic construct; identical to SEQ ID NO: 273

SEQ ID NO: 280 - protein, 133 aa, synthetic construct; identical to SEQ ID NO: 273

SEQ ID NO: 281 - protein, 118 aa, synthetic construct; identical to SEQ ID NO: 272

SEQ ID NO: 282 - protein, 160 aa, synthetic construct
MNTLFSRTLSKYYRMRLAHGFALWYNRYMKKLNKSRHARYLLNLHIVWIPKYRKAVLGPYREALTDILHSIAKDKGWEILAVEVMPDHIHLFVSVPPSISPSEVVKCFKGQSARKLLVEFPELVQQRDYRASFWAPSYFVASAGDVSSETIKAYIEGQYD

SEQ ID NO: 283 - protein, 143 aa, synthetic construct
MLRDFVMLYDRDGYHHSGHNKTKLLVHLIFVTKYRKAILAGDLRNAVKQQVFESAKRHLWYIDTMETDKDHIHILLQYPPDDSVRRIASTLKQESTYYIWKQYPEFLKQNYWKEHTLWSDGYFAASIGDVSTATIRKYIENQG

SEQ ID NO: 284 - protein, 135 aa, synthetic construct; identical to SEQ ID NO: 127

SEQ ID NO: 285 - protein, 118 aa, synthetic construct
MYHVIFVCKYRKKILEPLNAELKQTIHDIEKESDFEILEMETDKDHIHMLIKSEPKVSVVSIVRKLKQETTNRLWKSQGNYLKRYYWGEKTLWSDGYFASTIGNVSKEIAEAYIRNQG

SEQ ID NO: 286 - protein, 143 aa, synthetic construct; identical to SEQ ID NO: 283

SEQ ID NO: 287 - protein, 132 aa, synthetic construct
MSYFSKNRHKFYLKCHLIFVCKYRKQLLLSDIRDCVISVFKEVESKCDFDIEIMETDKDHIHLLINYPPNITVTSIVRILKQVSTNRLWKEFQPMLRKHFWKEHSFWSDGYFVCSIGEASPDIIRKYIENQG

SEQ ID NO: 288 - protein, 133 aa, synthetic construct
MDQYIRHNRHKYDLEVHLIFATKYRKQILKGALADDIKQKIYDICNRDGYSIVAMETDKDHIHILLRYGVTDRVCDIVKVLKQETTYYIWQRYSKFLRQCYWKKKIFWSDGYFACSVGKVSRATIKHYIDTQG

SEQ ID NO: 289 - protein, 132 aa, synthetic construct
MELESNNHSVFSLNYHLVMVVKYRRTVIDDTVSARLREIFEYIQPSYNISLKEWNHDRDHVHVLFSAHPKSELTKFLNAYKSASSRHIKKEFPEIREKLWKDYFWSRSFCLISTGGAPIETIRRYIESQGEK

SEQ ID NO: 290 - protein, 135 aa, synthetic construct
MDKVSIFAIGSDVTHGRGYVYCLQYHIVWCTKYRRKVIVGNIESDVKDHLYRTAEDLEIKILAIETMPDHIHMLIECKPQCRISDAIKVFKGNTARWLFLAHPEIKKQLWGGHLWDPSYFVATVSERTKEQITDM

SEQ ID NO: 291 - protein, 154 aa, synthetic construct
MLALDLNLRYNTRMEEIKYKSNNNVVYSCKYHVVFCPKYRRKVLQNGIDVRLKQLINECCEGIAVDILEIEIMPDHVHLLLEVDPQYGINKAVRHIKGYTSRVLRAEYPKLKTRMPTLWTNSYFVSTVGGAPLAIIKQYIENQKTSQRQKDKMG

SEQ ID NO: 292 - protein, 133 aa, synthetic construct
MENKYKRTATTVSLINYHIVFCPRYKRKIFNIPGVESRFKELVRKECSRRDIDIIAMECHVDHVHMFVSVYPQMAIPDILKYVKGATSHVLRDEFPQLKKMPSLWTRSYFVSTAGNVSSETIEWYVKTQKTRS

SEQ ID NO: 293 - protein, 136 aa, synthetic construct
MSVEAHHGRGYVYCLHYHIVWCVKYRHKVLTPEIVKDLVQILEKIAIDNNFEILEFNTDQDHVHILIDCSPQHFIPDIMKAIKGVSARLLMKKHGDILKKKLCGGHLWNPSYFITTISKNTEEQVSEYIKSQGKKD

SEQ ID NO: 294 - protein, 156 aa, synthetic construct
MIISSQQIGVKEMTEHMYHKNRGQDYYTNRHSCFLLQYHMVLVTKYRHPVITGEIKDLLYDKVKNIFTERKLNILELNGGEDHIHILYEADPFTSPGELANVVKTQTSRTVRKAYGSTALKKWYWKPYFWSESYFVTTVGINSLENVKNYIKNQGQ

SEQ ID NO: 295 - protein, 130 aa, synthetic construct
MEYKSNHNVVYSCKYHVVWCPKYRRSVLINGVDVRLKELIKETCDQLNVEIIEMEIMPDHVHLLMEVDPQLGVHKAVKQIKGYSSKMLRTEFPWLKSRLPSLWTNSYFVSTVGGAPLAVIKQYIENQKNV

SEQ ID NO: 296 - protein, 107 aa, synthetic construct
MSPKVETYLKKLVQSIAEEKVFTVSEVECGEWDHCHCFISAPPKLSVTQIMKYLKGITGRKLFEEFPELRAKLWKGHLWNHSYYVETIGSISEDVIRKYIEHQSKQY

SEQ ID NO: 297 - protein, 139 aa, synthetic construct
MSDGYTHKPGIVYKNQFHIIWCPKYRRKVLVDGVDIRLKEILLEEAAKMDVTIKSMEVMPDHVHLFIEFDPRLMEHKIIKTLKGVSAHKLREEFPWLKSRIPTLWTRSYFSCSVGHISEETITRYIEAQKRSEYWKQKP

SEQ ID NO: 298 - protein, 144 aa, synthetic construct
MERNFVHLNKTDGYHHRGHSKTRLLVHLVFVTKYRKTILFDSFSADVKQMLFESAKHHHWYIQTMETDKDHIHILLQYPPGDSIRHIVSILKQESTYHIWRTCHHKMLSQHYWAEHTLWSDGYFAASVGDASAETIRQYIASQG

SEQ ID NO: 299 - protein, 155 aa, synthetic construct
MFTGYSESMGDFRTGRHVVYSLHAHIVLVPKYRRKIMHGRVAEIVEAVTREVCDRRGVVLEAFNTDQDHCHLLVAYPPKVSLSSLVGSIKTMTSRAIHDANLAEVQHALRGPAFWSPSYCVVSCGGAPLEVVAQYVRDQGREPRKQGRQPRSAAD

SEQ ID NO: 300 - protein, 142 aa, synthetic construct
MSPRWTPDPNVRRGRSVVHNLHAHLVFTTKYRKAVFTDDLLTSCENAMRKVCADAGAELREFNGEGDHVHLLVQYPPQVQLSTLVNSLKGVSSRVLRRDHPQDVKQVLWGDHLWSPSYFAGSCGGAPLAIVKDYIENQKRPP

SEQ ID NO: 301 - protein, 134 aa, synthetic construct
MGGNLNRNQHSVYILTYHLVLVIKYRRKVINEDIFDSLMVIFEDIGFKYGVVVKEANWEVDHIHILFEAKPSTDLVKFINSYKSASSRIIKKQYPVIKKSLWKSAFWKTGYFITTTDGANIETIRQYIERQQEK

SEQ ID NO: 302 - protein, 180 aa, synthetic construct
MLSFQLKLSLNMAETKRNHAVYDLTYHIIFVTKYRRPVLSDEIGERLKTEITRLINEMKGEVKEIETDRDHVHILASLSPREAVTKQINVLKGVTARILRRDYGEELSQQLWGDSLWSESYYIATAGGVTIETLEKYVRSQKNGRSQAKICEIRKIREKASLQAGRFIYVLKGIVFSAFL

SEQ ID NO: 303 - protein, 108 aa, synthetic construct
MYEKNLIHYRTSVCNINYHVVWSVKYRRKVLNSKVETYLKDIVQQIADEKGFTVQMFECGEQDHVHCFITAPPKLSVTTIVKYLKGITGRKLYEQFPEIRSQLWKGQL

SEQ ID NO: 304 - protein, 131 aa, synthetic construct
MKNLKHNTTSVANINYHIIWCPKYRRKVLVDEIETRLKELLPELAKESGSTIDTMEVMPDHIHIFLKGTPTIPIHLIVKNLKGKSSRILRAEFPHLRKRLPCLWTRSYYCETIGCINEETIKKYIEDQKFN

SEQ ID NO: 305 - protein, 142 aa, synthetic construct
MNKDIVNIRNKSLAYGRGYVYSIQYHIAWCTKYRHRVLSGEVADALKGHLQTIADDYGFSITALEVMPDHVHMLVDCRPQFHPSDMVKIIKGNTARWLFMAYPQLKGQLWGGHLWNPSYCIVTVSDRSLEKVEQYIKTQKER

SEQ ID NO: 306 - protein, 142 aa, synthetic construct
MFTENRINSLNELSYGRGYVYLLRYHVVWCTKYRREVIKGSVYMDMLDYLQKSAEQIGVTLEEVELMPDHVHLLIKADPQVYIPNMIKILKGNTARWSFMSHPEIKAELWGGHLWNPSYFIATVSDRNTDQIREYIRNQKKK

SEQ ID NO: 307 - protein, 143 aa, synthetic construct
MCMSRNTNHYTKDRTCVYNVNYHIVWCVKYRNDILDAEIEQALRRILDEVAVENGFSIADVQVGEKDHVHCFVQAPPKLSITFIVKHLKGTSAIRLFREFPRLRETQRKHQLWNPSYFVETIGSTSEENVRRYIASQSKTVKG

SEQ ID NO: 308 - protein, 133 aa, synthetic construct
MDKYQHRRHNKNLLMVHLILVTKYRKKIFSGKFNDDIKQYIYDTCKKYHWYIKRMETDKDHVHILLQYNPTDTITGIVSCLKQQSTYLAWKDHSKYLTSHYWQRQVLWSSGYFAASIGQVSQAMIEHYIENQG

SEQ ID NO: 309 - protein, 133 aa, synthetic construct
MEVTHGRGYVYSIQYHIVWCVKYRHKIITEQIENRLIEILNKIADDNGFQILECNTDKDHIHLLVNCSPQHYIPDMIKALKGVSARLLMKEFGEELKRKLWGGHLWNPSYFVATVSENTEEQIRKYIQNQKRK

SEQ ID NO: 310 - protein, 132 aa, synthetic construct
MKNEIRRTNTTVSAVTYHFVFLPRYRRKILVDTVAERLWEVLEEACDEVECKIVALEIMPDHVHLFVSALPQWSPSAIMGQVKGKTSRILRAEFPALARMTSLWTRNFYVGSTGVASTETVRRYIAAQKTRG

SEQ ID NO: 311 - protein, 136 aa, synthetic construct
MSKKSNYISTNRSKHYLKCHLIFVCKYRKPMLVGQLNDDIKQIFQSIANGSDFEIEVMETDVNHVHFLVRYIPRLSIAQIVRRLKQESTRQIWLLHPSTLRKQYWYQHLLWSDGYFVCSIGEASPDTIRQYILSQG

SEQ ID NO: 312 - protein, 136 aa, synthetic construct; identical to SEQ ID NO: 311

SEQ ID NO: 313 - protein, 138 aa, synthetic construct
MTHYNDIRHGRHCVFNMHVHLVFVTKYRRGVFTKGILDEMRVIFANTCIDLEAELIEFDGEDDHVHLLVNYPPKLSVSILVNTLKGVSSRLIRKAKHPSITQKLWGGSLWSPSYFAASCGGAPIAIIRQYIEQQQTPH

SEQ ID NO: 314 - protein, 139 aa, synthetic construct
MTQETDYRRGRHCVFAIHAHLVFVTKYRRGVFRQEHLDSLHRILADVCKDFDAELIEFNGERDHVHLLINYPPKVALSALVNSLKGVSSRLLRKEFGDFHPWLKRRGVLWSPSYFAASCGGAPIEILRQYIEQQQAVPA

SEQ ID NO: 315 - protein, 142 aa, synthetic construct
MDIRTGRHCVLRLHTHLIFITKYRGNVFNEASLQRLEIIFKEICEAFEVEFTEFNGECDHVHLLVHYPPKVQLSKLINSLKGVSSRKLKQAFVYLREHWICKKSGSLWSPSYFAGSVGGAPLSVLKQYIEQQNRSLQSACRR

SEQ ID NO: 316 - protein, 134 aa, synthetic construct
MQVININKSRYAAYLLHVHLVFVTKYRKKILGDLHLAAFNQCAAEVCRDFGAELKECDGEADHVHMLIEYGPTVQLSVLVNSLKAVTSRRLRNEFLDLRGAYSKPVLWSRSYFASSCGGAPLDVIKQYIQNQRG

SEQ ID NO: 317 - protein, 134 aa, synthetic construct
MQVININKSRHAAYLLHVHLVFVTKYRKKILGDLHLAAFNQCAAEVCRDFGAELKECDGEADHVHMLIEYGPTVQLSVLVNSLKAVTSRRLRNEFLDLRGAYSKPVLWSRSYFASSCGGAPLDVIKQYIQNQRG

SEQ ID NO: 318 - protein, 133 aa, synthetic construct
MQYKLDKGNHSVYSIQFHYVACIKYRRKVLYGELVDRLKAINIDVARSFGINIVEQETAEDHIHILFSSKPQIQISKFVNSMKSVSARLIFKEFPYVKEILRKGHFWSPSYFLASTGQVTLDDIKHYIENQNA

SEQ ID NO: 319 - protein, 107 aa, synthetic construct
MTITNRDSTRQTSTVRKLKCEPHPLPFVQDSPDGTCSFWVVNPRVCNQWKTHLTEFEGEEDHVYLLINYPPKLALFLLVNRLKGVSSRVIRQQQYPSIQRQFWGGTL

SEQ ID NO: 320 - protein, 154 aa, synthetic construct
MYDWRTGRSCVFKNHAHLVFVTKYRREVFTAEMLERTKSIIAETCEQMDCELLEFSGEHNHIHLMVAIHPKVSVSNLVGKLKGKSSYMLRREFWGQIKTMLWGNHFWSPSYCVVSCGGATLEVIKQYIKEQNAPPSEQAVQRSKALTKTNLPPA

SEQ ID NO: 321 - protein, 133 aa, synthetic construct
MSHYISTNRSKHYLKCHLILVTKYRRNILVGQLNDDLKSIFTSIANNSDFEIEVFESDINHIHFLIRYIPRLSISQIARRLKQESTRQLWLLHHSTLRQYYWYQHILWSDGYFVCSIGEASPETIRQYILNQG

SEQ ID NO: 322 - protein, 134 aa, synthetic construct
MQMKIIHHRTCVFSIGYHLVFCTKYRRKVLTPKVDAVMKRVVAEMGIEKGFDVSTIETGPDHVHLFVSAPPHVSISDLVKWIKGISARKLLVAFPDLKKRMGSHLWNPSYYVGTVGDMSADVVRKYIENQKEKA

SEQ ID NO: 323 - protein, 136 aa, synthetic construct
MQDDIRRGRHVVYRLHVHLVFVPRYRRKVFDLDALERLRATLAAVCEDFGATLDEFNGEPDHVHLLVHYPPKVAVSVLVNSLKGVSARRLRQERPDIAKRYWSGGLWSASYFAASCGGAPIEVLRQYIEQQRAPTS

SEQ ID NO: 324 - protein, 131 aa, synthetic construct
MPQSHRHQSTSVSLVNYHLVFCPKRRKKILVDRVEARLEALVRGKAGKLDCAALELAVRPDHVHLIISCPPTLAPHQVARRIKGYTSRVLRQEFAYLKRLPSLWTRSYFVSTAGNVSSATIARYIEEQDTR

SEQ ID NO: 325 - protein, 136 aa, synthetic construct
MSKKSQYTSTNRSKHYLKCHLILVTKYRRNILVGQLNDDLKSIFSSIADNSDFEIEVMESDINHIHFLIRYIPRLSISQLVRRLKQESTRQLWLLHPTTLRQYYWYRKLLWSDGFFVCSIGEASPETIRQYILTQG

SEQ ID NO: 326 - protein, 136 aa, synthetic construct
MKYRSDKGSHSVYNLQFHYVACVKYRRKVLSDQVSDRLKEINLSVAKEAGVKIIEQETDRDHIHILFSIKPGIQLSKFINSLKSVSSRLLFREYPELKQQLWGGAFWSPSYFLASTGQVTLDVLKRYVENQNAKDL

SEQ ID NO: 327 - protein, 135 aa, synthetic construct
MEKIKGIRQGRNCVSELYVHLVFVTKYRRGVLTESMLSSLERYFCKVCGDFEAELLECNGEDDHVHLLISYPPKASISKMVNSLKGASSRLLRRDFPEIQEKLWQNALWSPSYFAASCGGAPLERIRQYIENQRS

SEQ ID NO: 328 - protein, 124 aa, synthetic construct
MRSTRHSRYNINYHLVWIPKYRRPVLTAKVAADLKEILENIADSKGVDILGLEIMPDHIHLFVSSPPQHAPSLLVNWFKGISARIYNHRWGAPYIRWTNSYYVGTAGTVTSETIKRYIEEQKSK

SEQ ID NO: 329 - protein, 136 aa, synthetic construct
MRYKLNNNVVYACKYHVVWCPKYRRKVLVDEVAVRLKELILQEAIALNAEIIEMEIMPDHVHLLIEVDPQFGIHRAIKQIKGKTSFSLRKEFKSLTTRLPSLWTNSYFVSTVGGAPLSAIKQYIENQKVSERKKVQ

SEQ ID NO: 330 - protein, 107 aa, synthetic construct
MFVRDAAERLRASFANVCKDFDAKLVERDSEHDHVLPLLKYPLKVAVSALVNNLKGISSRRLRKDRSALAACYWNGALWSLSYFAASCGEAPISVVRQYIKRQRTPK

SEQ ID NO: 331 - protein, 137 aa, synthetic construct
MENKYTHARTCVYNINYHIVWCVKYRRKVLSADISNRLIELLKAVGNEKGFSVVECKVGENDHVHCFVSAPPKISVTQIVKYLKGISGNQLFREYPQLRKSLWKGQLWNGSYFCETIGSTSEENILRYIERQKDCQL

SEQ ID NO: 332 - protein, 130 aa, synthetic construct; identical to SEQ ID NO: 211

SEQ ID NO: 333 - protein, 136 aa, synthetic construct
MSKKSNYITTNRSKHYLKIHLIFVCKYRKPLLMGNLNEDMKTILENITNNSDFSIEVMESDINHIHFLIRFIPRLSVSSIVRKLKQESTKQIWSLYSDVLSQEFWSEKTFWSDGYFACSIGEASPETIREYILNQG

SEQ ID NO: 334 - protein, 130 aa, synthetic construct; identical to SEQ ID NO: 211

SEQ ID NO: 335 - protein, 140 aa, synthetic construct
MNTIYKISNNVIYSCKYHVVWCPKYRRKILTDGVDVRLKDLLLEYAKKISVDVIEMDILPDYIHLLIEVDPQLGIHKAIKSLKGYTSKILRSEFSFLRTKLPTLWTNSYFVTTADNVSLEIIKKYIESQKTSQRQKEKLG

SEQ ID NO: 336 - protein, 133 aa, synthetic construct
MELDTNAHSVFSLHYHLILVVKYRRQVFTDEISNRAKEIFEYIAPTYKIELVEWNYDKDHVHVLFKGQPKTELSKFINAYKSASSRLLKKEFPTIRQKLWKEMFWSRSFCLLSSGGAPIEVIRQYIENQGKEK

SEQ ID NO: 337 - protein, 147 aa, synthetic construct
MSASGERFTHGRTSVYNLNYHIVWCTKHRRKILKNGIDSDLKAILREIADEHGYSIPHMEVGSDDHIHLFVSAPPKISVSTMVKQLKGTSSLRLFAMHPELKSQYWRSKGERSLWSPSYCVESIGVVNEQAVAEYIDSQRLKERESK

SEQ ID NO: 338 - protein, 139 aa, synthetic construct
MELDTNAHSVFSLYYHLILVTKYRKKVFDDEVSNRARELFEAIAPNYKIEVLEWNHEQDYIHVFFKAQPKTELSKFINAYKSASSRVLKKEYPSMKKQLWKEMFWSRSFCLLSSGGAPSEVIEQYIEEQGQKNDKTKGL

SEQ ID NO: 339 - protein, 147 aa, synthetic construct
MSTSGERFTHGRTSVYNLNYHIVWCTKHRRKVLKDGIDSDLKAILREIADERGYSILHMEVGLGDHIHLFVSAPPKISVSTIVKQLKGTSSLRLFAMHPELKSQHWRSKGERSLWSPSYCAESIGAVNEQAAAKYIDSQQLKEHEPK

SEQ ID NO: 340 - protein, 127 aa, synthetic construct
MYKARISYHIVFVTKYRRKVLSPNILELVKEVLEYAASKHNFSIIAFGSENQDHVHLVIEARPTQSVSKLVQLMKQYTRYHAWQKYAPWLRKFYWYRNFLWSSGYFCSSLGNVSKETILEYVERQNE

SEQ ID NO: 341 - protein, 133 aa, synthetic construct
MENKYRKTSTTISLINYHFIFCPRYRRKIFDISNVENRFKELVKDICEELDIKIIAMECDRDHTHMFLNCLPTLSPRDIMQKIKGVTSRELRKEFVELSKMPSLWTRSYFVSTAGNISSETVKQYVENQKKRY

SEQ ID NO: 342 - protein, 148 aa, synthetic construct
MVNRNEEVYSRRNHAKHRVFYHIIFSTKYRRRCLDGIMPELKTSFKRAEKMSNGLWKIEMVETDKEKIDHVHFLIRAVPQISPGEIIHKLKQFSTFDVWHNEKCREILSKFYWKNKKHGHILWTHGYFCSSVGEASSETIRRYISSQG

SEQ ID NO: 343 - protein, 139 aa, synthetic construct
MKVNYFKTRRATFNLTVHIVLVTKYRRKVFQKEHLEFLKVAFNSIAAKWDAEVVEINGESDHVHILLTYPPHKLLSGLIANLKSISCKLLWDNYPDHLKKIYGQDKRVLWTGAYFVASCGGVTIDQLKKYVENQDSPDF

SEQ ID NO: 344 - protein, 138 aa, synthetic construct
MCMKENLIHYRTSVCNINYHVVWSVKYRRKILNAQVEKYLKDVVQQIADEKGFIVQMFECGEQDHVHCFITAPPKLSVTTIVKYLKGITGRKLYEQFPEIRNKLWKGQLWNHSYYCETIGSVSEENIKRYIEKQSKSY

SEQ ID NO: 345 - protein, 131 aa, synthetic construct
MDLDSNNHSVFLLHYHMVLVVKCRRKVIDNIISNRLKEIFEYIQPNYNITLQEWNHDKDHVHVLFKAHPNTEISKFINSYKSASSRLIKKEFPQIRKYLWKEYFWSRSYCLLTTGGAPTEIIKKYIENQGK

SEQ ID NO: 346 - protein, 136 aa, synthetic construct
MTVETKHYRTCVANVNYHFVFVIKYRRKVLDDDLATTLKAVLNDIAMNKGFTIKAMEAGEQDHIHLFVTAHPKHSIGQIVKWLKGISGRVLLYRNKELARKLYRGHLWSSTYYVETIGSTSEDNIRRYIENQNYRG

SEQ ID NO: 347 - protein, 191 aa, synthetic construct
MSCYRRLGCSFKSKKFSVIFLNEIKNIFSKICYNNAMNIEYDKNLHSVFLLRYHFILVVKYRKQLINNKICDRLKDIFENIAKGQNYQLKIIEFNHDLDHLHILFKAKPKSELLKFINAYKSASSRLIKKEFPEIKKYLWKECFWSGSYFLATTGGVTLETLKQYVQNQGLEDNRVKKEYNSKKKRLLNNV

SEQ ID NO: 348 - protein, 129 aa, synthetic construct
MDYQRDEHRVHLIVYHIIFCPKRRKAVLGGDIARDCERLVRQKCAEKGWHVLQLAIQPDHVHLFVRVFPTTSAAEVVKEVKGLTSHELRQKYPVLHRLPSMWTRSYFAATAGNVSEETIRRYIEAQKGV

SEQ ID NO: 349 - protein, 136 aa, synthetic construct
MIETRIAVYDFHFHLVFVTKYRKSIFDTTEKQEELKTLLASFAEKNGSTIESVEVMPECVHLVLSFPPKFAPSSIVKSFKGAAAREWFKLHPEDKQKLYKGHLWNPSFFMQTVGVVSREMMMEYIENQLTKEPCRY

SEQ ID NO: 350 - DNA, 100 bp, synthetic construct
  source 1..100: mol_type = other DNA
ccagaccgcgccgcagtgctccgcctgtaaccgcatagcggttcagggggcggtgacgcggcgctatcgccgcagggcgatcactccggcctttcctgtt

SEQ ID NO: 351 - DNA, 100 bp, synthetic construct
  source 1..100: mol_type = other DNA
ccgggcgggaaccgtccgaggtgtggagcggtagaagtttcgccgcgatgaagcacgaaactcacgccatagccgtctaggcttggcgggagcagttcat

SEQ ID NO: 352 - DNA, 100 bp, synthetic construct
  source 1..100: mol_type = other DNA
tccagtgcgcggcaaacctccccgttcagggggaggtcgagagcgcggcggggaggcggttcaatgccgccgtgcccgcccttgctgggtgcaaccctaa

SEQ ID NO: 353 - DNA, 100 bp, synthetic construct
  source 1..100: mol_type = other DNA
tcgaagcgactgaagtagggccgctgctggcaggaaggaatcctcgcccttcccgctctcaagagcggcagccatcggctgagggcgaggaggatgtcaa

SEQ ID NO: 354 - DNA, 100 bp, synthetic construct
  source 1..100: mol_type = other DNA
tatccactcctacctaaagtagcaggcatgaacgaaagtttatgcggagtttggaagccccgtctatattcgcgaaagcggattaggcggggagggttca

SEQ ID NO: 355 - DNA, 100 bp, synthetic construct
  source 1..100: mol_type = other DNA
tgaaaacaaacattttaccaaggcccgcaggctccgtctatagcgacaagcgctaactttggctacgcttgtcgtttaggcggggttagtgctccttatg

SEQ ID NO: 356 - DNA, 100 bp, synthetic construct
  source 1..100: mol_type = other DNA
acaaagagcactgccgcaattttgcggcagcgtttttttatcacgcgtggcgtaatacccctgcctttaggcatggggatataagccacacattgttccc

SEQ ID NO: 357 - DNA, 100 bp, synthetic construct
  source 1..100: mol_type = other DNA
cccaaacctatacgctcggggacaccgtgtaagacctcgctggtgcaggcggtggtggctgaaccgagaatcccccggctttagccgtggggagtgtcaa

SEQ ID NO: 358 - DNA, 100 bp, synthetic construct
  source 1..100: mol_type = other DNA
cttaaaccgtatttatgatagaggaaactccgtttataacgataatagtttaaacggacagggtttcaccgtttatattacagataatcctgaaattcaa

SEQ ID NO: 359 - DNA, 100 bp, synthetic construct
  source 1..100: mol_type = other DNA
tttgaataaaatcataaccaacacgcattccatgcctatagttcattaaaacttaggtatggttagtgggttgtcatattgtgggtttattctgattctc

SEQ ID NO: 360 - DNA, 100 bp, synthetic construct
  source 1..100: mol_type = other DNA
cgtatggtcagaaaaggaagtctgtcattgggacgaagctgaaaccgcggaacccgacgacagaagcgctacccaaatatctttgggtggtgaggcttca

SEQ ID NO: 361 - DNA, 100 bp, synthetic construct
  source 1..100: mol_type = other DNA
taacgccgagtgcattctccatccaaacgaagtttgggtggagtaagcgaggcagactagttaattccttggttcttaatgtagacagtcactgtgtcgt

SEQ ID NO: 362 - DNA, 100 bp, synthetic construct
  source 1..100: mol_type = other DNA
cactcttattgattaacaaaaggcgcacacctcttccaaagcagtttgggagaggttagcctttttccttattcctctggtttcgtatatactcggtaat

SEQ ID NO: 363 - DNA, 100 bp, synthetic construct
  source 1..100: mol_type = other DNA
tcagtttctgagtcttggccccacggtggaaatgagtctggggcctaaacccccggaagctccatccatagcgttcacgcttgggtggtgaggttcactc

SEQ ID NO: 364 - DNA, 100 bp, synthetic construct
  source 1..100: mol_type = other DNA
ccagaacgcgccgtagtgctccgcctgtaaccgcatagcggttcaggaggtggcgaagcggcgcgatcacccaacgggcgatcattccggtctttcctgt

SEQ ID NO: 365 - DNA, 100 bp, synthetic construct
  source 1..100: mol_type = other DNA
tgcccgaggtgtggagcggggtaagaccacgggaaaccgtggctcctgcgtagaagcacgaaactcacgccataccgtaaggttggcgggagtagttcat

SEQ ID NO: 366 - DNA, 100 bp, synthetic construct
  source 1..100: mol_type = other DNA
acgtccggacgttcccatgtttatcgtttagttgttgggcgctcggcgcagcgtagctgcgccatcgcttgtgacagttgcgtaagtcctgagttctcaa

SEQ ID NO: 367 - DNA, 100 bp, synthetic construct
  source 1..100: mol_type = other DNA
tggaaacagaagaagccccacatctgacctgtactcaggcagtgtgggatgaattctgggcaatgacgaattgaccaacgagcggcattgaacgcagttc

SEQ ID NO: 368 - DNA, 100 bp, synthetic construct
  source 1..100: mol_type = other DNA
ccaggtgcgcgacaagccccgccgttcagggcggggaaggatagcgcggatgccgcaggtatccttgctttggatgctttcctagattggcgtctgctgt

SEQ ID NO: 369 - DNA, 100 bp, synthetic construct
  source 1..100: mol_type = other DNA
ccgctccgtgaagcaggaacccgccgaagcgactactcatgggctttcccttgtttagcgcggtaggaatccccggcattcatgccggggaggacgtcaa

SEQ ID NO: 370 - DNA, 100 bp, synthetic construct
  source 1..100: mol_type = other DNA
aaatctgccgcgcaagcccctggctttagccatggggtgagcggcagccggtttgttatctgatacggaacttgtataatgtttccatgtcagaatataa

SEQ ID NO: 371 - DNA, 100 bp, synthetic construct
  source 1..100: mol_type = other DNA
ggatgttgtagccacgtcgctccggcgacagtggcgagagccggaaagacccctatcaccggcaccagaaccccccggctttagccgtggggaggttcag

SEQ ID NO: 372 - DNA, 100 bp, synthetic construct
  source 1..100: mol_type = other DNA
aatgaaaaccgtaggaactacggagttagcctggataaacttatctcattagagatattgactaggaagcccatcgagctttagctcgtgggtagttcac

SEQ ID NO: 373 - DNA, 100 bp, synthetic construct
  source 1..100: mol_type = other DNA
agaaatcccattagctttagctgatgggatgaatgacagagaataattatatatgaataataattggaaatctaaaaacagacataaatatttattacaa

SEQ ID NO: 374 - DNA, 100 bp, synthetic construct
  source 1..100: mol_type = other DNA
aaagaaaacgatgcaaccctctttctttagagggaggttagtcgcgcttccagtgccgcaggcacgcctttttctgtttcaagatttgattgcgttgcgg

SEQ ID NO: 375 - DNA, 100 bp, synthetic construct
  source 1..100: mol_type = other DNA
cggccgttttcgacgatggaggaatcgggacactcccgggtgtctaaggggctgctttgcagcctacgaagctcgttggctttagccgcgagtagttcac

SEQ ID NO: 376 - DNA, 100 bp, synthetic construct
  source 1..100: mol_type = other DNA
gcccatcagagttaaactttggtctaactctgtgcaagaatctccccactttttagatggcgcaagcgtagcgagagccgtctagtggggagagtgtcaa

SEQ ID NO: 377 - DNA, 100 bp, synthetic construct
  source 1..100: mol_type = other DNA
gcgggagcttggtagccccgtttttcaaaacggggtggaaaagcgacacgagcggatttatccgccgtcaaaatcccaagacattcaatgccgctcgtga

SEQ ID NO: 378 - DNA, 100 bp, synthetic construct
  source 1..100: mol_type = other DNA
atggttctcctttgtctgcgcttctgtttgtggacattctcagtataacacagttggggtgggtgtacaacaatcccgcacatacgcgcgactcggtcgg

SEQ ID NO: 379 - DNA, 100 bp, synthetic construct
  source 1..100: mol_type = other DNA
gaaacgtcaagcctgtcactatccccgagattcatgccacacggcaatgcctcggggaataaacagaatcccgtccattcacggacgggagcacgtcaaa

SEQ ID NO: 380 - DNA, 100 bp, synthetic construct
  source 1..100: mol_type = other DNA
atagaaaccgcaatggtggggcttttgtcatgtgcccctacaagcagatgggactttttacaatcaaggaaagttactttcctggataacccactttctg

SEQ ID NO: 381 - DNA, 100 bp, synthetic construct
  source 1..100: mol_type = other DNA
ccgttaagcgaggaaccgggcaatgcgttaacgcgttgctaccctacaaagggaatccccttcctgattcacgtagtgagaagctgggggaggatgtcaa

SEQ ID NO: 382 - DNA, 100 bp, synthetic construct
  source 1..100: mol_type = other DNA
caaccttctttggagagatatagtaagacctgctattttagtaggcaagtatcgtttgtatccaagaatcccttgcctttaggcatggggagtatgtcaa

SEQ ID NO: 383 - DNA, 100 bp, synthetic construct
  source 1..100: mol_type = other DNA
gatataagctcgctaaaaaacaatgattcgatatcattgttttttattgtattatatttttaatagttatataatacttttatgaaatataaatcaaata

SEQ ID NO: 384 - DNA, 100 bp, synthetic construct
  source 1..100: mol_type = other DNA
actctcttttagagcttcggcctgcgaagcgcaatctccgggctcccggcttttcaaccttcgagaagcctcgggtctattgacccgggaagtatgtcac

SEQ ID NO: 385 - DNA, 100 bp, synthetic construct
  source 1..100: mol_type = other DNA
caaagtgaacgggaaaacctcgggtttcgaacccggggatgaaagtgaacaccttattctcccgtacgctctcaaaatattaccgcatgcgccttgcaca

SEQ ID NO: 386 - DNA, 100 bp, synthetic construct
  source 1..100: mol_type = other DNA
acccatttggcctcacaccattcccaccatagcccttgcctttgtctcttgacaacgccgcatggctatccggcattggaactcctgagtcatcacaaac

SEQ ID NO: 387 - DNA, 100 bp, synthetic construct
  source 1..100: mol_type = other DNA
cgttttgcgcagcaggccggaggccctgtaagaccagcttgctggcttcggtttgtgaagcgtcaagaatccctgccctttagggcagggagtatgtcaa

SEQ ID NO: 388 - DNA, 100 bp, synthetic construct
  source 1..100: mol_type = other DNA
cgggatgttgtagccccgccccctggcggtggcgagagcccgggagaggttgcggtggttccccttagaacccccgggctttagccctggggaggctcag

SEQ ID NO: 389 - DNA, 100 bp, synthetic construct
  source 1..100: mol_type = other DNA
gaccgagaaacggccgccgcaggccccgccctttagggctggggataaggcggccaaatttttatgggctacaattatatttccgcttgtgctataataa

SEQ ID NO: 390 - DNA, 100 bp, synthetic construct
  source 1..100: mol_type = other DNA
aaatatccttcggcttggactgagccgtcaggcgctaacgcaacgggttgccgtaagcgtagcctgagaagctgacgcctttaggcgtcagagtggtcac

SEQ ID NO: 391 - DNA, 100 bp, synthetic construct
  source 1..100: mol_type = other DNA
aggactcctcctctcagttcaggctgatcgtttgctgcgcaacgggctcctgccagggatccccagcccaccataaaaccttagcggcgtggggactgcc

SEQ ID NO: 392 - DNA, 100 bp, synthetic construct
  source 1..100: mol_type = other DNA
ccacattaaataaagttacagaaacatagccagaaagctcaaatgctttagcgttgagatgaaacaggaagcctcactgctttagcgtgaggtagctcac

SEQ ID NO: 393 - DNA, 100 bp, synthetic construct
  source 1..100: mol_type = other DNA
gatcataagcgagaattctctcctttttaaaggagagatgaaagcgagtatctgtttttttttaatgctttattttttttaagtgatttttcttttatgt

SEQ ID NO: 394 - DNA, 100 bp, synthetic construct
  source 1..100: mol_type = other DNA
aaccccttgcaaagaaaaccccgttctctaagacggggatgaatttgcaactctttacattcagtcgtaattatagtataataccattatgaaagcaata

SEQ ID NO: 395 - DNA, 100 bp, synthetic construct
  source 1..100: mol_type = other DNA
aggaactatccttatggacttaaaacagaaagcctttcgctcagtctcattggagattgttagttaggaagcctccacctcttaggtgggggtagttcac

SEQ ID NO: 396 - DNA, 100 bp, synthetic construct
  source 1..100: mol_type = other DNA
agccagtggagtgtgctatgacggggagtttaaattgttaaactctagttatcatactatgaaactggaatctcgtcactttagtgatgtagaggttcaa

SEQ ID NO: 397 - DNA, 100 bp, synthetic construct
  source 1..100: mol_type = other DNA
aggtggatataatgtgaaatatcgatttaggtttcccgaatttgatgttccattcttcgcacactcgtgactttagtcgtgagtaagaagaatatatgtc

SEQ ID NO: 398 - DNA, 100 bp, synthetic construct
  source 1..100: mol_type = other DNA
atatacttgaccgtgggatgcacggcaacaatgctttacttaaaatagctgtttaggctatggatgaagaatccaccttattcatggggtggagtatcaa

SEQ ID NO: 399 - DNA, 100 bp, synthetic construct
  source 1..100: mol_type = other DNA
tatcaaatcgtagcgtaaaaccacctcttttaagtggtggatataagctacatctgctttggcagatattagtcaggcgttttttgttgttcaatatact

SEQ ID NO: 400 - DNA, 100 bp, synthetic construct
  source 1..100: mol_type = other DNA
catgctttgtccgcaaacccccgccgttcagggcggggatgtaagcggcacaatccacgcaagtaattattcaggcgttctttgttgttcaatgtactga

SEQ ID NO: 401 - DNA, 100 bp, synthetic construct
  source 1..100: mol_type = other DNA
tgtgtagtgaatggtgcagtcatgccgtcagcagcagaaaccagtgagcagtagcgatacctactcactcttaggaaactaggaatccccttccttcagg

SEQ ID NO: 402 - DNA, 100 bp, synthetic construct
  source 1..100: mol_type = other DNA
acatttttagaagaggtcaggaactgacctttgttggaggagatacaatagtatcttcaatgaatcaagaatctatgtcctttagggcatagtagttcaa

SEQ ID NO: 403 - DNA, 100 bp, synthetic construct
  source 1..100: mol_type = other DNA
ataaggtgtggttgggtgtgttaaggtgcagtaagtcaaggcaaggattttcatattcataattatgtggcgagttgagtttgggtcagttagggtacgg

SEQ ID NO: 404 - DNA, 100 bp, synthetic construct
  source 1..100: mol_type = other DNA
attggcagagccgaaaaccacgtaacttcagttcgtggatgaaggcaagcagcgcttttaagcgcctgtcttttaatgggtactgtgatgggggtcatta

SEQ ID NO: 405 - DNA, 100 bp, synthetic construct
  source 1..100: mol_type = other DNA
tacaggagttggagtctctactcttcttggtgaaaacctggttgagcaaattctgacgtagaatgtagaatccccgtctcttcagagcggggagtgtcaa

SEQ ID NO: 406 - DNA, 100 bp, synthetic construct
  source 1..100: mol_type = other DNA
tccagtgtgtcgtaaaaccccttgctttagctatggggatataagacacttccatcgaatttacgcaagtaattgaagatggaacaaaatatccttgtat

SEQ ID NO: 407 - DNA, 100 bp, synthetic construct
  source 1..100: mol_type = other DNA
ttgtggacactgtgtaagacattgagataccgaatggtatcagccaatgcagtagtggttgaagcaagaatccccctgctttagctgtggggagtgtcaa

SEQ ID NO: 408 - DNA, 100 bp, synthetic construct
  source 1..100: mol_type = other DNA
actccattatctccaaataattctgaatatttcaaaaatcactttacaacctttatcacatcgtgccttgcatctatgttgtataatatttagccatatg

SEQ ID NO: 409 - DNA, 100 bp, synthetic construct
  source 1..100: mol_type = other DNA
gaaggattaagacttttggcttcctgacaggaagagatatacgtagggcggggcacgcccgaacggtgcagaaatgcacagcgcccggggaaaccgtgta

SEQ ID NO: 410 - DNA, 100 bp, synthetic construct
  source 1..100: mol_type = other DNA
aagttcttgggcgcagacccccgacttcagtcgtggggttagcctaaatctccgatccccagcattagatagctacaagctgtatactgttggggtggct

SEQ ID NO: 411 - DNA, 100 bp, synthetic construct
  source 1..100: mol_type = other DNA
ttgggcggtccgtgaagcgccaaccacccgagaccgaggctgcatgaacggcacctcagccacgggaatcccccaccttcaggtgtggggaggaagtcaa

SEQ ID NO: 412 - DNA, 100 bp, synthetic construct
  source 1..100: mol_type = other DNA
caaaagaatgccgtaaagcccctgcctttaggcatggggatataaggcatatcatttatattaatttgcatttgcattatacagctatgtcctgtataat

SEQ ID NO: 413 - DNA, 100 bp, synthetic construct
  source 1..100: mol_type = other DNA
gctgaatattaaaaccctcgggcagagggttcaggcgctaacggatgggttgctgtccgttgcctgagaatccccttgattcatcaatggggagtgtcaa

SEQ ID NO: 414 - DNA, 100 bp, synthetic construct
  source 1..100: mol_type = other DNA
cagcatcatctcctcacacgaggatccaccatcaaaacctcccgaggggactgtcgccttcaggcgacgggtgaatcggggtcccgccagggactggccc

SEQ ID NO: 415 - DNA, 100 bp, synthetic construct
  source 1..100: mol_type = other DNA
gaacgcggcggtaaacatcttgagggcagggcttgcccttcgcgaggctgcggaagcggcttagcgagaagccgctcccttcagggagcggaggagtcac

SEQ ID NO: 416 - DNA, 100 bp, synthetic construct
  source 1..100: mol_type = other DNA
agtaaaggcaagaaaacccactcatctttagtgggtgggatgaattgccttaaccttggttctcaatatattttctcactgtatctggatttgcttcacc

SEQ ID NO: 417 - DNA, 100 bp, synthetic construct
  source 1..100: mol_type = other DNA
ggactgtcgattacactgatggagcagatgtaattcctaatattaggttatcagcaatgaaatcagaagcccacaaatctttagtttgtgggtagttcac

SEQ ID NO: 418 - DNA, 100 bp, synthetic construct
  source 1..100: mol_type = other DNA
atgaagtgtcgccgcacactcgtgactttagtcatgagttaggcggcacataacatatgtcattgtaaaatcacaaataaaaccttgaaataatttagct

SEQ ID NO: 419 - DNA, 100 bp, synthetic construct
  source 1..100: mol_type = other DNA
tgccctatcgtagaaagacagtccgctgtcctaaagacagcatgacacgaacttaatcgtgtcgttggaagcacccgactttagtcgggtgagggttcac

SEQ ID NO: 420 - DNA, 100 bp, synthetic construct
  source 1..100: mol_type = other DNA
ccgaaccgatacgctcggggagaccatgtaagacctcacacggtgcaagcaacggtcagcgaaccgagaatcccccggctttagccgtggggagtgtcaa

SEQ ID NO: 421 - DNA, 100 bp, synthetic construct
  source 1..100: mol_type = other DNA
cttatcgcgtgtggcgtaataccccttcctttaggcatggggatataagccacaccttgttccctcgtaaaaagacttccggggattgtgttagatgccg

SEQ ID NO: 422 - DNA, 100 bp, synthetic construct
  source 1..100: mol_type = other DNA
aaacgcttgcggagagggcgtaagaccttagctactttagtgggtaaggcaatcctcggtgaagcaagaatcccctttctttagatatggggagtgtcaa

SEQ ID NO: 423 - DNA, 100 bp, synthetic construct
  source 1..100: mol_type = other DNA
tgcgggcaacgagtatgtccagacgcttgattttgaagaatttggctatccaaattttaaggcgtatgaagaattgagtgaagcaactatggggatataa

SEQ ID NO: 424 - DNA, 100 bp, synthetic construct
  source 1..100: mol_type = other DNA
ctttctgatcaagagtgtcccgtcctttagggcgggagtgaattgaccaactacgcaggtgtatcctgctgctcaatgtagcggcggacggtgtcgattc

SEQ ID NO: 425 - DNA, 100 bp, synthetic construct
  source 1..100: mol_type = other DNA
ggcttgccggagcagtttcccgcgaagcaggaacccaccgaagccgccgggaaaccggggcagtaggaatccccgtcctttagggcggggaggatgtcaa

SEQ ID NO: 426 - DNA, 100 bp, synthetic construct
  source 1..100: mol_type = other DNA
atgaagtgtcgccgcacactcgtgactttagtcatgagttaggcggtacataacatatgtcattgtaaaatcacaaataaaaccttgaaataatttagct

SEQ ID NO: 427 - DNA, 100 bp, synthetic construct
  source 1..100: mol_type = other DNA
aatagtgtaacctctgttggttagtcagctaatcaataagtctgcactctacccaagaagcccctacctcttagcgtatgcgtaggtaggggtggttcac

SEQ ID NO: 428 - DNA, 100 bp, synthetic construct
  source 1..100: mol_type = other DNA
actaacagcacgtaattcccccacctcataggtggtgggatgtaagtgcttcggtcttgtgcagggctagaagccctgtgcgtgaccgacaaatagaaag

SEQ ID NO: 429 - DNA, 100 bp, synthetic construct
  source 1..100: mol_type = other DNA
acttgtgtaaccgctgttggttagaaagctaatcatcaagtcagcacattacccaagaagctcctacctcttagcgtatgcgtaggtaggagtggttcac

SEQ ID NO: 430 - DNA, 100 bp, synthetic construct
  source 1..100: mol_type = other DNA
gacttgctccctgtgcgtgaccgacaataaagaggattggcaagtttttgactttccttttgtcttatgataaaataaagtcagttatagttcgtaaaag

SEQ ID NO: 431 - DNA, 100 bp, synthetic construct
  source 1..100: mol_type = other DNA
ctttctatttgtcggtcacgcacagggcttctagccctgcactagaccgaagcacttacatcccaccacctgagaggtgggggaattacgtgctgttagt

SEQ ID NO: 432 - DNA, 100 bp, synthetic construct
  source 1..100: mol_type = other DNA
gtgaaccacccctacctacgcatacgctaagaggtaggggcttcttgggtagagtgcagacttattgattagctgactaaccaacagaggttacactatt

SEQ ID NO: 433 - DNA, 100 bp, synthetic construct
  source 1..100: mol_type = other DNA
acgtaatcatgcaagaattctcccacctctgcaggtggtgagatgaattgcataccttgttctcccgtatcaccattgttgcatgttccataagagtatg

SEQ ID NO: 434 - DNA, 100 bp, synthetic construct
  source 1..100: mol_type = other DNA
ggggcacacggggatagctcgttgatacttagccggttgcggctattgagcgagaagcccccgcctcaacacgtcagtgtaggcggcgggagtatgtcac

SEQ ID NO: 435 - DNA, 100 bp, synthetic construct
  source 1..100: mol_type = other DNA
gttgttcattgatgaattgtcgatgcacacttgtgacttcagtcatgagtaaggcgacgcataacatatgtcattaaaaaaacacgaataataccatata

SEQ ID NO: 436 - DNA, 100 bp, synthetic construct
  source 1..100: mol_type = other DNA
tgctttatcgtaggaaggcagtccgctgttctaaagacagtatgacatgaacttaatcgtgtcgttggaagcgcctgattttagtcgggtgagggttcac

SEQ ID NO: 437 - DNA, 100 bp, synthetic construct
  source 1..100: mol_type = other DNA
ccgaatgtcgccgtaaagccccttgctttagcaatggggatataaggcgaccattatattgctaaaagttatctaattattattgaactagataggtcgt

SEQ ID NO: 438 - DNA, 100 bp, synthetic construct
  source 1..100: mol_type = other DNA
tacccacaccttgcactgcgacctaacagaaatgtattggtcagccgataacatcggcaggtagcaagaatcccctgccttcaggcatggggagtgtcaa

SEQ ID NO: 439 - DNA, 100 bp, synthetic construct
  source 1..100: mol_type = other DNA
aggacgctggaccggtcaacaacccggaccatcgtcaacccctagccgtttaaccaacggaactaggaatcccctcccggaagggaggggaggatgtcaa

SEQ ID NO: 440 - DNA, 100 bp, synthetic construct
  source 1..100: mol_type = other DNA
ctggctcacaaccccaccgacaccaccatcacaaaaccaaatacccctaaaaaaaaaaccaaatatcgccctacaacaaaacaactaccccaaccaaaac

SEQ ID NO: 441 - DNA, 100 bp, synthetic construct
  source 1..100: mol_type = other DNA
atgttcctgaaaaggttctgttatacagtactgacaaaggaaagacgggtttccccgtttggacatggaagcacgcgacttcagtcgtgtgaggcttcac

SEQ ID NO: 442 - DNA, 100 bp, synthetic construct
  source 1..100: mol_type = other DNA
ttgttttatcattccacagtttcaaaacggtaggcataccttccgcttcattatctttgattgatgcactggcgcattctcgtgactttagtcgtgagta

SEQ ID NO: 443 - DNA, 100 bp, synthetic construct
  source 1..100: mol_type = other DNA
aacacgaccgcgacgtgaatgccgccatgaacatcgccgcgcgcggacatgcgcgtctcgcaggaggaatccccgtcctttagggcggggaggatgtcaa

SEQ ID NO: 444 - DNA, 100 bp, synthetic construct
  source 1..100: mol_type = other DNA
ccggacgaagcgacaagccccggggtttagcccggggtagagcgttcaagccggaacggcttgctgctgctcgatgtactggcgcaggatttcgattggt

SEQ ID NO: 445 - DNA, 100 bp, synthetic construct
  source 1..100: mol_type = other DNA
accaaggaagccgtaaagccccttcctttaggtaggggatataaggctatcggcggcacgccgattgaagcgatctattttgttgttcaatatactgctt

SEQ ID NO: 446 - DNA, 100 bp, synthetic construct
  source 1..100: mol_type = other DNA
tgcatgatagagatattaatgccgccaagaacattctcgcgctcggacatgagcgtctggcagcaggaatccgcgccctttagggcggggagaatgtcaa

SEQ ID NO: 447 - DNA, 100 bp, synthetic construct
  source 1..100: mol_type = other DNA
cccgatgaagcaggaaccaggtactgcgcgagcagctatcacccagtaatgggaatccccctcctgagacgcgaagcgcgaaggtgggggaggatgtcaa

SEQ ID NO: 448 - DNA, 100 bp, synthetic construct
  source 1..100: mol_type = other DNA
gctatcaaccgcgccccacagtaccggcaaaaggtcagcagcaaggggagcgctcagcaccagcgaaaaggcaagcagagaccaccttttttgacgaaag

SEQ ID NO: 449 - DNA, 100 bp, synthetic construct
  source 1..100: mol_type = other DNA
actaatacgccgggaagtccccttccgaaaggtgggggatgaaaggcgttttcccttgacagtagctgtcagttctgttataatacaagcatatgaaata

SEQ ID NO: 450 - DNA, 100 bp, synthetic construct
  source 1..100: mol_type = other DNA
taccgcaggaatctgtggaaagttacgcctggggagagtcggcgcagtcgggcctctcgttgaaccaggaagccccttccgtaagggaggggtagttcac

SEQ ID NO: 451 - DNA, 100 bp, synthetic construct
  source 1..100: mol_type = other DNA
ccctctcgccgtaaaaccccgtcgttaagggcggggatataaggcggctaacttcgctggttttcgatgtattgccgaatcctctccagcggggctcccc

SEQ ID NO: 452 - DNA, 100 bp, synthetic construct
  source 1..100: mol_type = other DNA
cctcgatgaagcaggaacccaccgaaatccaccaaacggaacgcgcttccgcttgagggatcgtaggaatcctcttccttcagggaggggaggaggtcaa

SEQ ID NO: 453 - DNA, 100 bp, synthetic construct
  source 1..100: mol_type = other DNA
gttgtattccacttggttgttgtcatgtctttttgtttatttaccgtttaggtatttttttgtttgattttatttaccgatttttgataaatataccaaa

SEQ ID NO: 454 - DNA, 100 bp, synthetic construct
  source 1..100: mol_type = other DNA
tgtcaatgaccgacgctgagaaaattttaaggttgaagcaagaaaatctctggcgacatcagccagaatctcaacggtctttagccgttgagtagttcaa